(19) United States
(12) Patent Application Publication
(10) Pub. No.: US 2017/0202878 A1
(43) Pub. Date: Jul. 20, 2017

(54) Method for Preventing or Treating a Protein Aggregation Disease

(71) Applicant: Queen Mary University of London, London (GB)

(72) Inventors: Denise Schwarz, London (GB); Johan Aarum, London (GB)

(21) Appl. No.: 15/326,553

(22) PCT Filed: Jul. 17, 2015
(86) PCT No.: PCT/GB2015/052087

§ 371 Date: Jan. 16, 2017

(30) Foreign Application Priority Data
Jul. 18, 2014 (GB) 1412802.9

(51) Int. Cl. A61K 33/24; C12Q 1/68; A01K 67/027; A61K 31/713

(52) U.S. Cl. CPC A61K 33/24; A61K 31/713; C12Q 1/6883; A01K 67/0275; C12Q 2600/118; A01K 2267/0306; A01K 2207/05; A01K 2207/20; A01K 2207/10

(57) ABSTRACT

The present invention relates to a method for treating and/or preventing a disease associated with protein aggregation which comprises the step of preventing protein aggregation associated with RNA removal, by stabilising RNA; or reversing protein aggregation associated with RNA removal, by effectively replacing removed RNA.

### FIELD OF THE INVENTION

The invention relates to a method for treating and/or preventing a disease associated with protein aggregation which comprises the step of preventing protein aggregation associated with RNA removal, by stabilising RNA; or reversing protein aggregation associated with RNA removal, by effectively replacing removed RNA. The invention also relates to methods for diagnosing a disease or determining if a subject is at risk of developing a disease which is associated with protein aggregation. The invention also relates to an animal model for a disease associated with protein aggregation.

### BACKGROUND TO THE INVENTION

The assembly of proteins into insoluble aggregates is a hallmark of several diseases, including many neurodegenerative diseases such as Alzheimer's disease (AD), Parkinson's disease (PD), Huntington's disease (HD), amyotrophic lateral sclerosis (ALS) and prion diseases. Protein aggregation, however, is by no means restricted to the central nervous system (CNS) and also occurs in diseases as diverse as Type II diabetes and Inclusion body myositis/myopathy.

Each of the relevant neurodegenerative diseases involves selective neuronal vulnerability with degeneration in specific brain regions and deposits of abnormal proteins in neurons, other cells or extracellularly. It is increasingly recognised that these neurodegenerative diseases have common cellular and molecular mechanisms including protein aggregation and inclusion body formation. The aggregates usually consist of fibres containing misfolded proteins which may have a β-sheet conformation, and there is partial but not perfect overlap among the cells in which abnormal proteins are deposited and the cells that degenerate.

Although each disease is primarily associated with the aggregation of a specific protein, there is a considerable overlap and the same protein may be found to aggregate across a variety of diseases. For example, AD is primarily associated with aggregated amyloid-β and tau proteins, PD with aggregates comprising protein α-synuclein bound to ubiquitin and HD with mutant Huntingtin. It has been reported, however, that although α-synuclein aggregates are invariant characteristics of PD, they also occur in AD. Similarly, TDP-43 aggregation is associated with ALS and frontotemporal dementia but also with many (30-50%) cases of AD.

Despite the well-reported association between protein aggregates and neurodegenerative diseases, the causative mechanisms leading to the generation of aggregates remain elusive. A consequence of the poor understanding of the processes involved in the generation of protein aggregates and the subsequent neurodegenerative disorders with which they are associated is an absence of curative therapeutic strategies. There is currently no curative treatment for any neurodegenerative disease associated with protein aggregation. As such, current treatment strategies focus on palliative care and aim to repress the appearance of symptoms for as long as possible.

In most neurodegenerative disorders the familial mutations are extremely rare and the majority of cases occur without any family history. Current methods to study protein aggregation rely largely on the use of recombinant proteins in vitro or the forced expression of proteins, frequently harbouring familial disease-causing mutations, in cells or model organisms. While these methods may suffice for the study of single proteins they rarely replicate the aggregation of all proteins associated with the particular disease. For example, transgenic animal models of AD (transgene expression of mutated APP and/or PSEN1 or PSEN2) do show amyloid-β aggregation but do not demonstrate tau aggregation and thus lack one of the hallmarks of human AD. Currently, to replicate tau aggregation, mutations that have never been found in human AD must be introduced into the MAPT gene. The failure rate of drugs targeting neurodegenerative diseases is high, despite the fact that some drugs, for example for AD, demonstrate efficacy in animal models of disease.

There is thus a need for alternative therapies and methods for treating diseases associated with protein aggregation. There is also a need for alternative models of diseases associated with protein aggregation which are not associated with the disadvantages described above.

### DESCRIPTION OF THE FIGURES

FIG. 1. RNA gel electrophoresis after RNAse treatment Human neuronal cell lystate was treated with various concentrations of RNAse, the remaining RNAs separated on an agarose gel and stained with ethidium bromide.

FIG. 2. Protein aggregation after treatment with various enzymes. Cell lysate from human neurons (A) or mouse brain cortex (B) were treated for 1 hour at 37° C. with the indicated enzymes. Aggregated proteins were collected by centrifugation and analysed by coomassie SDS-PAGE.

FIG. 3. The effects of different RNases on the aggregation of selected proteins. Lysate from human neurons was treated with the indicated ribonucleases for 1 hour at 37° C. and the aggregated proteins used for western blot analysis.

FIG. 4. Protein re-folding and aggregation. Jurkat cell lysate was treated with RNAse A/T1 and aggregated proteins collected by centrifugation. The pelleted proteins were denaturated in guanidine hydrochloride and mixed with total Jurkat RNA (+) or water (−). The mixtures were then dialysed overnight against TBS. An aliquot was taken from each sample (total) for SDS-PAGE and aggregated proteins collected by centrifugation (pellet). The supernatant (sup) was then treated with RNAse A/T1 or vehicle (Ve−) and aggregated protein collected by centrifugation (pellet). All samples were separated on SDS-PAGE and proteins stained with coomassie.

FIG. 5. Prion-like propagation of aggregation. Western blot analysis of Huntingtin (HTT), FUS and TARDBP aggregation. Jurkat cell lysates were treated at 37° C. for 15 min with immobilised RNAse A (biotinylated RNAse A coupled to magnetic strepavidine beads) or Ve− (strepavidine beads). After treatment, 10% of the lysate were mixed with non-treated lysate and incubated for 1 hour at 37° C. Aggregated proteins were isolated by centrifugation, solubilised in SDS and separated on SDS-PAGE gels. Blots were probed with antibodies against the indicated proteins. The increase in aggregation seen in the RNAse treated samples is likely to be due to propagation of the aggregation, e.g. recruitment of native proteins in the non-treated sample.

FIG. 6. Removal of RNA causes protein precipitation. a, Schematic diagram depicting the general experimental setup. Soluble cell-free lysates are treated with ribonucleases for one hour at 37° C. and then centrifuged to separate aggregated (pellet) and soluble proteins (supernatant). b,c Coomassie staining of SDS-PAGE separated proteins. Cell lysates from human neurons (b) or mouse cortex (c) were treated for one hour at 37° C. with increasing concentrations of a mixture of RNAse A and RNAse T1 (A/T1). Aggregated proteins were pelleted by centrifugation and solubilised in SDS. An equal volume from each sample was separated on SDS-PAGE, and proteins stained with coomassie stain. d, Analysis of protein aggregation after incubation with different ribonucleases or DNAse I. e, Assessment of protein aggregation after co-treatment of cell lysate with RNAse A and an RNAse A inhibitor (RNasin). f, Examination of protein aggregation following the addition of RNAse- or alkaline hydrolysed RNA. All gels were stained with coomassie. Each experiment was performed at least twice on different cell and tissue preparations with high reproducibility.

FIG. 7. Computational analysis of RNAse-precipitated proteins. a, Top five gene ontology classes by location (top) or molecular function (bottom). b, Cumulative distribution of the proportion of predicted low-complexity regions or unstructured regions (top figure) in the RNAse-aggregated proteins (Red) or random sets of proteins (Blue).

FIG. 8. Degradation of RNA induces precipitation of proteins associated with neurodegenerative diseases. a. Western blot detection of protein aggregation. Human neuronal cell lysate was incubated at 37° C. for 1 hour in the absence (Ve−) or presence (RNAse A/T1) of an RNAse A/T1 mixture. Aggregated proteins were collected by centrifugation (Pellet) and soluble proteins collected in the supernatant (Sup). Proteins were separated by SDS-PAGE, transferred to membranes and probed with the indicated antibodies. b, Western blot analysis of soluble and precipitated proteins in lysate from HEK293 cells expressing GFP-Abeta or GFP. c, Inhibition of RNAse A diminishes the precipitation of indicated proteins. The amount of RNasein represented by the 1× concentration inhibits approximately 50% of the added RNAse A (based on manufacture's data). d, Western blot analysis of RNAse-precipitated proteins from lysate prepared from mouse cortex. e, Schematic diagram of the re-folding assay where RNAse-aggregated proteins are solubilised in 6M guanidine hydrochloride and then allowed to re-fold in the presence or absence of RNA. Soluble and aggregated proteins are then separated by centrifugation and the soluble fraction treated with RNAse A/T1 or vehicle to induce protein re-aggregation. f, Coomassie stained gel showing the global protein profile of soluble (Sup 1) and aggregated (Pel 1) proteins after re-folding in the presence (+) or absence (−) or total RNA. After removal of aggregated proteins the soluble fractions (Sup 1) were treated with RNAse A/T1 (A/T1) or vehicle (Ve−) to examine protein re-aggregation (Pel2). Proteins remaining soluble after treatment with RNAse or vehicle are observed in the lanes marked Sup 2. Asterix (*) denote added RNAse A. g, Western blot analysis of indicated proteins after re-folding in the presence or absence of total RNA. h, Assessment of the capacity of total human RNA (hRNA), total E. coli RNA, yeast tRNA, human genomic DNA (gDNA), or heparin to re-fold RNAse-aggregated proteins. All samples were treated with the same amount (in weight) of indicated nucleic acids or heparin. All experiments were performed at least twice with high reproducibility.

FIG. 9. Ribosomal RNA is required for the solubilisation of RNAse-aggregated proteins in vitro. a, Gel-electrophoresis analysis of co-precipitated RNA following immunoprecipitation (IP) from crosslinked cells with antibodies against the prion protein (PrP) or non-specific IgG antibodies (IgG). Total RNA (lane 1) was loaded as a reference and the various ribosomal RNA species are indicated on the right. b, Graphic representation depicting the alignment of cDNA clones obtained after immunoprecipitation of indicated proteins refolded in the presence of total human RNA. c, PAGE-Urea gel-analysis of RNA samples (1 μg) used in d and e to assess their capacity to solubilise RNAse-aggregated proteins. d, Assessment of the capacity of the various RNA samples shown in c to re-fold huntingtin (HTT), neurofilament heavy chain (NF-H), or PrP. e, The same experiment as in d but examining the global protein profiles by coomassie staining after re-folding. All experiments were performed at least twice with high reproducibility.

FIG. 10. Protein aggregation after addition of pre-hydrolysed RNA.

Lysate from human neurons was treated with increasing amounts of RNAse A- or NaOH hydrolysed RNA and aggregated proteins analysed by western blot.

FIG. 11. Protein solubilising effect of predicted and non-predicted G-quadruple-forming oligos. RNAse aggregated proteins from human neurons were solubilised in 6M guanidine hydrochloride and mixed with the indicated oligos. After removal of guanidine hydrochloride through dialysis, aggregated proteins were collected by centrifugation and quantified. Ve− represents TE buffer, 18S 1129 represents a predicted G-quadruple forming sequence derived from the 18S ribosomal RNA. No 4G 1129 contains the same nucleotides as 18S 1129 but in an order not predicted to form G-quadruples.

FIG. 12. Inhibition of RNAse mediated protein aggregation by sodium orthovanadate. Lysate from human neurons was treated for one hour at 37° C. with a constant amount of RNA A/T1 (0.1 μg/100 μg protein) and increasing concentrations of Sodium Orthovanadate. Aggregated proteins were collected by centrifugation and the amount of proteins in the pellet determined.

FIG. 13. Riobonucleoside vanadyl (VA) causes protein aggregation. Lysate from mouse cortex was treated with vehicle (Ve−) or VA (10 mM) for one hour at 37° C.

Aggregated proteins were collected by centrifugation and analysed by SDS-PAGE gel electrophoresis and stained with coomassie.

FIG. 14. Genomic DNA refold RNAse-aggregated proteins. RNAse aggregated proteins were solubilised in 6M guanidine hydrochloride and mixed with vehicle (Ve−, TE buffer), total human RNA, or human genomic DNA. Guanidine hydrochloride were removed by dialysis and aggregated proteins collected by centrifugation (P1) and quantified using the BCA assay (Therma Scientific).

FIG. 15. ATP-hydrolysing activity of RNAse aggregated proteins refolded with human total RNA. A) Analysis of ATP-captured proteins after refolding B) RNAse aggregated proteins from human neurons were solubilised in 6M guanidine hydrochloride and mixed with RNA. After removal of guanidine hydrochloride the soluble proteins were asses for their ATP-hydrolysing activity in the presence (+) or absence (−) of RANse A/T1 using the ADP assay (Promega). Data is expressed in arbitrary units (AU). *p<0.05. C) RNAse aggregated proteins from Jurkat T-cells were solubilised in 6M guanidine hydrochloride and mixed with RNA. After removal of guanidine hydrochloride the soluble proteins were asses for their ATP-hydrolysing activity in the presence (+) or absence (−) of RANse A/T1 using the ADP assay (Promega). Data is expressed in arbitrary units (AU). *p<0.05, **p<0.01.

FIG. 16. Divalent ions cause protein aggregation. A. Cell lysate was prepared from Jurkat T-cells treated with various concentrations of divalent ions (Mg²⁺, Ca²⁺, and Zn²⁺), and the aggregated proteins collected by centrifugation and analysed by gel electrophoresis and coomassie blue staining. Ve−=vehicle. B. Jurkat proteins re-folded with total Jurkat RNA treated with various concentrations of Mg²⁺ or a RNAse A/T1 mixture. Aggregated proteins were collected by centrifugation and solubilised in 2% SDS/8M Urea by sonication, and the amount of proteins was determined using the BCA assay (Pierce). C. Same as in (D) but the proteins were re-folded with genomic DNA instead of total RNA.

FIG. 17. Refolding of proteins with a synthetic RNA fragment. Aggregated proteins from Jurkat T-cells were solubilised in GuHCl and refolded with a synthetic fragment of RNA derived from a consensus sequence of human AluSx repeats. Total RNA and genomic DNA (gDNA) are included for comparison. The synthetic AluSx transcript is as efficient in refolding the proteins as total RNA. Pellet 1 represents the aggregated proteins after refolding, and pellet 2 represents the aggregated proteins when the soluble fraction after pellet 1 is treated with an RNAse A/T1 mixture. Proteins are analysed by SDS-PAGE and stained with coomassie blue.

FIG. 18. Sodium orthovanadate can mimic RNA/DNA in preventing protein aggregation after refolding. Aggregated proteins from Jurkat T-cells were solubilised in GuHCl and refolded in the presence of increasing concentrations of sodium orthovanadate. Aggregated proteins were collected by centrifugation and solubilised in 2% SDS/8M Urea by sonication, and the amount of proteins was determined using the BCA assay (Pierce).

FIG. 19. Comparison of the protein aggregating effect of Sodium orthovanadate and (NH4)[VO(O2)2(phen)]*2H2O. Cell lysate prepared from Jurkat T-cells treated with various concentrations of sodium orthovanadate or (NH4)[VO(O2)2(phen)]*2H2O (Comp 6). Aggregated proteins were collected by centrifugation and the amount of proteins was determined using the BCA method (Pierce).

FIG. 20. Fraction of peaks annotated to various genomic regions. Proteins were re-folded together with pre-fragmented genomic DNA and used for ChIP with antibodies against Abeta or the prion protein (PrP). Alternatively, all soluble proteins and associated DNA were captured by binding to a nitrocellulose membrane (All). DNA isolated from these samples was used to prepare libraries for next generation sequencing and sequenced on the Illumina platform. Peaks were identified by comparing the read densities from Abeta or PrP against an IgG control. For the “All” samples, the peaks were identified by comparing the protein associated-DNA with DNA retained on a nitrocellulose membrane from DNA-only input (without proteins). The diagram shows the fraction of annotated peaks in the most common regions. TSS=transcription start site, TTS=transcription termination site.

FIG. 21. Predicted motifs in Abeta peaks. MEME-ChIP was used to predict motifs in the binding regions (peaks) of RNA associated with soluble Abeta. The motifs represent the + strand and are displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 22. Predicted motifs in NFH peaks. MEME-ChIP was used to predict motifs in the binding regions (peaks) of RNA associated with soluble neurofilament heavy chain (NFH). The motifs (A-E) represent the + strand and are displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 23. Predicted motifs in PrP peaks. MEME-ChIP was used to predict motifs in the binding regions (peaks) of RNA associated with soluble prion protein (PrP). The motifs (A-E) represent the + strand and are displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 24. Predicted motifs in tau peaks. MEME-ChIP was used to predict motifs in the binding regions (peaks) of RNA associated with soluble tau (MAPT). The motifs (A-E) represent the + strand and is displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 25. Predicted motifs in DNA bound to refolded proteins (All). MEME-ChIP was used to predict motifs in the binding regions (peaks) of DNA associated with soluble proteins refolded and captured by binding to nitrocellulose membranes. The motifs represent the + strand and are displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 26. Predicted motifs in DNA bound to soluble Abeta. MEME-ChIP was used to predict motifs in the binding regions (peaks) of DNA associated with soluble Abeta refolded and captured by ChIP. The motifs represent the + strand and are displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 27. Predicted motifs in DNA bound to soluble PrP. MEME-ChIP was used to predict motifs in the binding regions (peaks) of DNA associated with soluble prion protein (PrP) refolded and captured by ChIP. The motifs represent the + strand and are displayed as DNA motifs. The reverse complimentary motifs are as likely to be associated with the proteins as the shown forward motifs. To generate the RNA versions of the motifs, the “T”s are changed to “U”s. Only the top 5 most significant motifs are shown.

FIG. 28. Synthetic oligomers of identified motifs mimic genomic DNA in preventing aggregation after refolding. Cell lysate prepared from Jurkat T-cells treated with various concentrations of the double stranded form of Motif 1 and Control 1 (A), or Motif 2 and Control 2 (B). Aggregated proteins were collected by centrifugation and solubilised in 2% S DS/8M Urea by sonication, and the amount of proteins was determined using the BCA assay (Pierce). Genomic DNA (gDNA) at 15 μg is included as a reference. Sequences of the motifs and controls are shown in Table 7. Data were normalised to refolding performed with Vehicle (Ve−). uM and ug refers to μM and μg, respectively.

### SUMMARY OF ASPECTS OF THE INVENTION

The present inventors have made the important finding that endogenous RNA has a role in maintaining cellular proteins in an aggregate-free state and that removal of RNA causes protein aggregation.

The present inventors have further determined that polyanionic molecules, such as RNA and genomic DNA, can be used to refold proteins which have been aggregated by RNA removal.

In a first aspect the present invention relates to a method for treating and/or preventing a disease associated with protein aggregation which comprises the step of preventing protein aggregation associated with RNA removal, by stabilising RNA; or reversing protein aggregation associated with RNA removal, by effectively replacing removed RNA.

The RNA may be stabilised by altering ion balance in the cell.

The RNA may be effectively replaced by adding RNA, DNA or LNA. The RNA may be ribosomal RNA. The DNA may be genomic DNA.

The RNA, DNA (e.g. genomic DNA) or LNA may comprise a G-quadruple structure (G4).

The RNA may be effectively replaced by sodium orthovanadate, or a derivative, structural mimic or modified version thereof.

The disease may be type II diabetes; cancer; inclusion body myositis/myopathy; medullary carcinoma of the thyroid, atrial amyloidosis, hereditary cerebral haemorrhage with amyloidosis, pituitary prolactinoma, injection-localised amyloidosis, aortic medial amyloidosis, hereditary lattice corneal dystrophy, corneal amyloidosis associated with trichiasis, cataract, calcifying epithelial odontogenic tumour, pulmonary alveolar proteinosis, cutaneous lichen amyloidosis, a nonneuropathic systemic amyloidosis, or a neurodegenerative disease such as Alzheimer's disease, motor neuron disease (MND), Parkinson's disease, frontotemporal dementia, amyloidosis lateral sclerosis, Huntington's disease, spinocerebellar ataxias, spinocerebellar ataxia, spinal and bulbar muscular atrophy, denatotrubal-pallidoluysian atrophy, familial British dementia, familial Danish dementia and prion diseases.

In a second aspect the present invention relates to a method for diagnosing a disease associated with protein aggregation which comprises the step of determining the level of effective RNA in a sample from a subject, wherein decreased effective RNA compared with an equivalent sample from a control subject indicates that the subject has, or is at risk of, a disease associated with protein aggregation.

In a third aspect the present invention relates to a method for determining if a subject is at risk of developing a disease associated with protein aggregation which method comprises the step of determining the level of effective RNA in a sample from the subject, wherein decreased effective RNA compared with an equivalent sample from a control subject indicates that the subject has, or is at risk of, a disease associated with protein aggregation.

The RNA may be ribosomal RNA. The RNA may comprise G quadruple structures.

The decrease in effective RNA may be due to RNA degradation.

The sample may be a serum, plasma, cerebrospinal fluid sample or a tissue sample such as a brain, pancreatic or muscle sample.

The disease may be type II diabetes; cancer; inclusion body myositis/myopathy; medullary carcinoma of the thyroid, atrial amyloidosis, hereditary cerebral haemorrhage with amyloidosis, pituitary prolactinoma, injection-localised amyloidosis, aortic medial amyloidosis, hereditary lattice corneal dystrophy, corneal amyloidosis associated with trichiasis, cataract, calcifying epithelial odontogenic tumour, pulmonary alveolar proteinosis, cutaneous lichen amyloidosis, a non-neuropathic systemic amyloidosis, or a neurodegenerative disease such as Alzheimer's disease, motor neuron disease (MND), Parkinson's disease, frontotemporal dementia, amyloidosis lateral sclerosis, Huntington's disease, spinocerebellar ataxias, spinocerebellar ataxia, spinal and bulbar muscular atrophy, denatotrubal-pallidoluysian atrophy, familial British dementia, familial Danish dementia and prion diseases; or Age-Related Macular Degeneration or Retinitis Pigmentosum.

In a further aspect the present invention relates to an animal model for a disease associated with protein aggregation, in which animal protein aggregation is induced by removal of RNA in a cell in the animal.

The RNA may be ribosomal RNA. The RNA may comprise G quadruple structures.

The RNA may be removed by inducing RNA degradation. The RNA degradation may be caused by administration of, or increasing the expression or activity of, an RNA ribonuclease.

The ribonuclease may comprise RNase A, RNase T1 and/or RNase 1f.

The effective amount of RNA may be reduced by administration of antisense RNA or siRNA.

The effective amount of RNA may be reduced by inducing a reduction in RNA expression.

In a further aspect the present invention provides an animal model for a disease associated with protein aggregation, in which animal protein aggregation is induced using ribonucleoside vanadyl and/or divalent ions. In one embodiment, the divalent ions comprise Mg²⁺, Ca²⁺, or Zn²⁺.

In a further aspect the present invention provides the use of ribonucleoside vanadyl to initiate the aggregation of a plurality of proteins in a cell or cell lysate.

At least one protein in the plurality of proteins may be implicated in the pathogenesis of a disease associated with protein aggregation. The disease may be Type II diabetes; Inclusion body myositis/myopathy; or a neurodegenerative disease such as Alzheimer's disease, motor neuron disease (MND), Parkinson's disease, frontotemporal dementia and prion diseases.

The plurality of proteins may comprise at least one of the following: amyloid-β, MAPT, SCNA, TARDBP, FUS, HTT, PrP, Neurofilaments (NF-H) and alpha-synuclein.

In a further aspect the present invention provides a method for promoting the folding of a protein in vitro which comprises the step of contacting an unfolded or partially folded protein with RNA or DNA (e.g. genomic DNA) in order to promote folding.

In a further aspect the present invention provides a use of RNA or DNA (e.g. genomic DNA) to promote the in vitro folding of an unfolded or partially unfolding protein.

The RNA or DNA (e.g. genomic DNA) may comprise a G-quadruple structure. The protein may be a therapeutic protein, or a biological reagent such as an enzyme, antibody, protein ligand, receptor, structural protein or cofactor. The protein may be a transmembrane protein.

### DETAILED DESCRIPTION

The present inventors have shown that RNA removal in a cell or cell lysate initiates protein aggregation and that polyanionic molecules, such as RNA and DNA (e.g. genomic DNA), can be used to refold proteins which have been aggregated by RNA removal.

The present invention relates to a method for treating and/or preventing a disease associated with protein aggregation which comprises the step of preventing protein aggregation associated with RNA removal by stabilising RNA; or reversing protein aggregation associated with RNA removal by effectively replacing removed RNA.

### RNA Removal

The term “RNA removal”, as defined herein, means a reduction in the total quantity of intact RNA molecules or a reduction in the total quantity of RNA which has a native (unaltered) structure. The term “RNA removal”, as defined herein, may also refer to disruption or removal of RNA-protein interaction.

RNA removal may occur due to a decrease in the total quantity of RNA which has a native (unaltered) structure (i.e. a loss of RNA structure integrity). The structure of RNA may be altered, for example, by a change (e.g. a reduction) in the levels of divalent ions (e.g. Mg²⁺, Ca²⁺, or Zn²⁺) within the cell, or a change (e.g. a reduction) in the level of divalent ions which are associated with RNA molecules. RNA removal may encompass a loss of secondary structure of RNA molecules, or a loss of tertiary or quaternary structure.

RNA removal may be or involve RNA degradation.

The term “degradation” is used herein in its conventional sense to relate to the destruction of the RNA. Destruction of the RNA may be achieved by the disruption of the primary structure of an RNA molecule via the cleavage of the phosphodiester bonds between adjacent nucleotides.

### Stabilising RNA

RNA structural integrity (e.g. RNA secondary structure) is dependent upon a number of factors, for example the levels of divalent ions (e.g. Mg²⁺, Ca²⁺, or Zn²⁺) within the cell and/or associated with the RNA molecules.

The method of the present invention may involve stabilising RNA in a cell in a subject by altering the ion balance in the cell. Such stabilisation enables the maintenance of RNA structures within the cell and thus reduces protein aggregation. As used herein, “stabilising RNA” may also refer to the stabilisation of RNA-protein interaction.

The intracellular ion balance of a cell can be altered through the administration of ion channel blockers (antagonists) and/or activators (agonists). For example, intracellular calcium levels can be altered by Flunarizine or Fuspirilene, both Ca-channel blockers. Potassium can be altered by the administration of for example Diazoxide, Minoxidil, or Nicorandil (all activators) or Amiodarone (K-channel blocker). Increased intracellular sodium can be achieved by the administration of Alpha-Pompilidotoxin or decreased through the administration of Quinidine, Lidocaine, or Encainide. Levels of copper and zinc could be reduced by the use of Clioquinol or its derivative, PBT2 (a Prana Biotechnology compound).

### Replacing Removed RNA

The present inventors have shown that the protein aggregation induced by the removal of RNA can be reversed by the addition of polyanionic molecules. These molecules are able to induce the re-folding of proteins which aggregated upon the removal of RNA and thus are capable of effectively replacing the RNA which has been removed.

In the context of the present invention, the RNA may be effectively replaced by a molecule which has the capacity to maintain, preserve or sustain proteins in a non-aggregated state or to facilitate the re-folding of proteins which have previously aggregated due to RNA removal.

The RNA may be effectively replaced by a polyanionic molecule, for example a nucleic acid such as RNA, ‘locked-nucleic acid’ (LNA), or DNA (e.g. genomic DNA [“gDNA”]).

The nucleic acid may be RNA. The RNA may be single- or double-stranded. The RNA may comprise, consist essentially of or consist of ‘locked-nucleic acids’ (LNA). The RNA may comprise, consist essentially of or consist of ‘peptide nucleic acids’ (PNA). The RNA may be a protein-coding RNA or a non-protein-coding RNA (e.g. a ribosomal RNA, transfer RNA, or repetitive RNA).

Molecules which are able to substitute for RNA in the promotion of protein folding and thus are capable of effectively replacing removed RNA can be readily determined using the RNAse-induced protein aggregation and refolding assay described in the Examples section herein.

In one embodiment, the RNA is effectively replaced by a nucleic acid aptamer, e.g. an RNA aptamer or a DNA aptamer. Thus, in one embodiment, the term “nucleic acid” includes aptamers. An aptamer may be a single-stranded nucleic acid that is able to bind to a specific molecular target with high affinity.

### Locked Nucleic Acid (LNA)

The ribose moiety of an LNA nucleotide is modified with an extra bridge connecting the 2′ oxygen and 4′ carbon. The bridge “locks” the ribose in the 3′-endo (North) conformation and this locked ribose conformation enhances base stacking and backbone pre-organization.

The LNA used in the method of the present invention may comprise, consist essentially of or consist of LNA nucleotide(s).

### Ribosomal RNA

The RNA may be ribosomal RNA.

Ribosomal ribonucleic acid (rRNA) is the RNA component of the ribosome, and is essential for protein synthesis in all living organisms. It constitutes the predominant material within the ribosome, which is approximately 60% rRNA and 40% protein by weight. Ribosomes contain two major rRNAs and 50 or more proteins. The rRNA in the large ribosomal subunit acts as a ribozyme, catalyzing peptide bond formation.

Most eukaryotes comprise an 18S rRNA in the small ribosomal subunit, whereas the large ribosomal subunit contains three rRNA species (5S, 5.8S and 28S).

The term ribosomal RNA or rRNA includes rRNA-like sequences which appear in other types of transcript. Many eukaryotic mRNAs contain sequences that resemble segments of 28S and 18S rRNAs which are present in both the sense and antisense orientations (Mauro and Edelman (1997) Proc. Natl. Acad. Sci. 94:422-427).

In the present invention removed RNA may be effectively replaced by rRNA, or a structural mimic thereof, to treat and/or prevent a disease associated with protein aggregation.

The rRNA may be, comprise, or consist essentially of ribosomal RNA.

The rRNA may be, comprise or consist essentially of 18S and/or 28S rRNA.

The rRNA may comprise at least 50%, 60%, 70%, 80%, 90%, 95%, 99% ribosomal RNA.

The RNA which is increased may consist of rRNA (i.e. be effectively 100% rRNA).

### DNA

The term “DNA” includes genomic DNA, and synthetic or non-naturally occurring DNA. The DNA may be a repetitive DNA element, for example a LINE (Long Interspersed Nuclear Element) or SINE (Short Interspersed Nuclear Element).

Genomic DNA (gDNA) refers to chromosomal DNA, in contrast to extrachromosomal DNAs such as plasmids.

### G-Quadruple Structures

The RNA, LNA or DNA (e.g. gDNA) may be capable of forming a G-quadruple/G-quadruplex structure(s).

G-quadruplexes (also known as G-tetrads) are nucleic acid sequences that are rich in guanine and are capable of forming a four-stranded structure. Four guanine bases can associate through Hoogsteen hydrogen bonding to form a square planar structure called a guanine tetrad, and two or more guanine tetrads can stack on top of each other to form a G-quadruplex. The quadruplex structure is further stabilized by the presence of a cation, especially potassium, which sits in a central channel between each pair of tetrads.

A wide range of sequences and motifs which are capable of forming G4 quadruplexes are known in the art, for example as described in Maizels N & Gray L T (2013) (The G4 Genome. PLoS Genet 9(4): e1003468. doi:10.1371/joumal.pgen.1003468).

G-quadruplexes may be intramolecular, bimolecular, or tetramolecular. Depending on the direction of the strands or parts of a strand that form the tetrads, structures may be described as parallel or antiparallel.

### Sodium Orthovanadate

Removed RNA may be replaced by providing, or increasing the amount of, a molecule which is able to substitute for RNA in the promotion of protein folding in a cell. An example of such a molecule is sodium orthovanadate, or a derivative, structural mimic or modified version thereof.

Sodium orthovanadate (Na₃VO₄) contains the tetrahedral VO₄³⁻. It is an inhibitor of protein tyrosine phosphatases, alkaline phosphatases and a number of ATPases, most likely acting as a phosphate analogue. The VO₄³⁻ ion binds reversibly to the active sites of most protein tyrosine phosphatases.

The present inventors have demonstrated that sodium orthovanadate is capable of inhibiting protein aggregation induced by RNA removal.

Examples of derivatives, structural mimics and modified versions of sodium orthovanadate include other chemical compounds comprising the tetrahedral VO₄³⁻ ion, for example potassium orthovanadate. Examples of vanadium complexes also include: ammonium (2,6-pyridinedicarboxylic)dioxovanadate (NH₄[V(O₂)(dipic]), bis(maltolato)oxovanadium (BMOV), bis(N′,N′-dimethylbiguanidato)oxovanadium (VO(metf)2.H2O), potassium oxalatooxo-diperoxovanadate (K3[VO(O₂)₂(ox)].2H2O), ammonium (2,2′-bipyridine)oxodiperoxo-vanadate ((NH4)[VO(O₂)₂(bipy)].4H2O), and ammonium (1,10-phenanthroline)oxodiperoxo-vanadate ((NH4)[VO(O₂)₂(phen)].2H2O).

### Pharmaceutical Composition

The RNA, LNA or DNA (e.g. gDNA) or sodium orthovanadate (or a structural mimic thereof) may be administered to a subject in need thereof in the form of a pharmaceutical composition.

As such, the RNA, LNA or DNA (e.g. gDNA) or sodium orthovanadate may be administered with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents.

Typically, a physician will determine the actual dosage which will be most suitable for an individual subject and it will vary with the age, weight and response of the particular patient. The dosage is such that it is sufficient to reduce and/or prevent protein aggregation.

Treat and/or Prevent

The method for the prevention of a disease associated with protein aggregation relates to the prophylactic implementation of the method for a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, a disease associated with protein aggregation.

A method for the treatment of a disease associated with protein aggregation relates to the therapeutic implementation of the method. Herein RNA is stabilised or removed RNA is effectively replaced in a cell of a subject having an existing disease or condition in order to lessen, reduce or improve at least one symptom associated with the disease and/or to slow down, reduce or block the progression of the disease.

### Use

The present invention provides RNA, LNA, DNA (e.g. gDNA), sodium orthovanadate or a structural mimic thereof for use in treating and/or preventing a disease associated with the presence of protein aggregates.

The RNA, LNA, gDNA or sodium orthovanadate or structural mimic thereof may be for use in a method as described herein.

### Protein

The term “protein” is used in the normal sense to mean a series of residues, typically L-amino acids, connected one to the other, typically by peptide bonds between the α-amino and carboxyl groups of adjacent amino acids. The term “protein” as used herein is synonymous with peptide or polypeptide.

### Protein Aggregation

The term “protein aggregation” refers to the biological phenomenon in which mis-folded proteins accumulate and clump together, either intra- or extracellularly. Mis-folded proteins may form aggregates because the exposed, hydrophobic portions of the unfolded protein interact with the exposed hydrophobic patches of other unfolded, or mis-folded, proteins, spontaneously leading to protein aggregation. The formation of mis-folded proteins into aggregates may be termed plaque formation.

The present invention provides a method to prevent and/or treat a disease associated with protein aggregation by stabilising RNA or by effectively replacing removed RNA in a cell, thereby reducing the formation of aggregates of proteins that require the presence of RNA for the maintenance of their native structure.

Some mutations result in the protein being particularly sensitive to mis-folding and aggregation. In the method of the invention, the protein may or may not comprise one or more such predisposing mutations.

One or more of the proteins may be ubiquinated after aggregation. Ubiquitination of proteins is a hallmark of several of the diseases mentioned below.

### Subject

The subject may be any individual suffering from, or at risk of, a disease associated with protein aggregation, for example a disease mentioned below.

The subject may be a mammal. For example the subject may be primate, mouse, rat, guinea pig or rabbit.

The subject may be a human.

### Diseases

The disease which is treating and/or prevented by the method of the present invention may be any disease which is associated with the presence of protein aggregates. A wide-range of such diseases are known in the art, as described by Chiti et al., Annu. Rev. Biochem. 2006. 75:333-66.

Both mature and immature protein aggregates may be toxic to cells. The hydrophobic patches of immature aggregates may interact with other components of the cell and damage them, whilst mature aggregates may disrupt cell membranes and cause them to become permeable.

The formation of protein aggregates is associated with a range of diseases and the subsequent toxicity of the protein aggregates may be mechanistically involved in the pathogenesis of the disease.

The method of the present invention is used to treat and/or prevent a disease which is associated with protein aggregation.

A number of diseases are associated with the formation of protein aggregates, including but not limited to a range of neurodegenerative diseases such as Alzheimer's disease (AD), motor neuron disease (MND), Parkinson's disease (PD), Huntington's disease (HD), frontotemporal dementia and prion diseases. In addition protein aggregation may occur in other diseases as diverse as Type II diabetes and Inclusion body myositis/myopathy.

Neurodegenerative disease refers to diseases characterised by the progressive loss of structure or function of neurons, including neuronal death. Many identified pathophysiological features may be similar between neurodegenerative diseases, particularly the appearance of protein aggregates and death of neurons.

The method of the present invention may be used to reduce the level of protein aggregation in a neuronal cell. The term ‘neuronal cell’ refers to a cell of the central nervous system. In particular the neuronal cell may be associated with a region of the central nervous system in which degeneration occurs during a neurodegenerative disease.

AD is the most common form of dementia and is commonly diagnosed in people over 65 years of age, although the less-prevalent early-onset AD may occur much earlier. AD is characterised by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Amyloid plaques, comprising beta-amyloid peptides and other cellular material, may be present outside and around neurons, whilst neurofibrillary tangles, comprising aggregates of the microtubule-associated protein tau, which has become hyperphosphorylated, may be present intracellularly. Lewy bodies may also occur in AD. The majority of cases of AD are sporadic, meaning that they are not genetically inherited although some genes may act as risk factors. On the other hand, around 0.1% of the cases are familial forms of autosomal dominant inheritance, which usually have an onset before age 65. This form of the disease is known as early onset familial AD.

In order to prevent and/or treat AD, the method of the present invention may reduce the aggregation of Abeta, MAPT and/or SNCA.

The method of the present invention may reduce MAPT aggregation and also reduce hyperphosphorylation of MAPT. Hyperphosphorylation of MAPT is a hallmark of AD and other tauopathies.

MND is characterized by rapidly progressive weakness, muscle atrophy and fasciculations and muscle spasticity. The pathophysiological features of MND may include the loss of both upper and lower motor neurons in the motor cortex of the brain, the brain stem, and the spinal cord. Prior to their destruction, motor neurons develop protein aggregates in their cell bodies and axons, which may contain ubiquitin, and generally incorporate one of the ALS-associated proteins: SOD1, TAR DNA binding protein (TARDBP) or FUS. Only around 5% of MND cases are associated with a familial history of the disease, however, mutations in several genes have been linked to various types of MND. Examples of these genes include, but are not limited to, SOD1, ALS2, FUS, ANG and TARDBP.

PD is characterized by the loss of dopamine-generating cells in the substantia nigra, more specifically the ventral part of the pars compacta of the midbrain. Early in the course of the disease, the most obvious symptoms are movement-related and include shaking, rigidity, slowness of movement and difficulty with walking and gait. Later, cognitive and behavioural problems may arise, with dementia commonly occurring in the advanced stages of the disease. Other symptoms include sensory, sleep and emotional problems. The loss of dopamine-generating cells may occur due to the formation of protein-aggregates comprising alpha-synuclein bound to ubiquitin which accumulate in the neurons and form Lewy Bodies. According to the Braak staging, a classification of the disease based on pathological findings, Lewy bodies first appear in the olfactory bulb, medulla oblongata and pontine tegmentum, with individuals at this stage being asymptomatic. As the disease progresses, Lewy bodies later develop in the substantia nigra, areas of the midbrain and basal forebrain, and in a last step the neocortex.

The method of the present invention may reduce aggregation of SNCA and reduce phosphorylation of SNCA. Phosphorylation of SNCA is associated with PD.

HD affects muscle coordination and leads to cognitive decline and psychiatric manifestations. The disease may be caused by an autosomal dominant mutation in the Hungtingtin (HTT) gene wherein a CAG trinucleotide repeat becomes expanded beyond a threshold level. The CAG repeat encodes a polyglutamine tract in the mature Huntingtin (Htt) protein and this tract may vary in length between individuals. Once the polyglutamine tract extends beyond a certain length, however, it causes the formation of a mutant Huntingtin (mHtt) protein which is unable to fold as required. This mis-folding leads to the formation of protein aggregates comprising the mis-folded mHtt.

Frontotemporal dementia may result from the progressive deterioration of the frontal lobe of the brain which may, over time, progress to degeneration of the temporal lobe. Frontotemporal dementia may be associated with the formation of protein aggregates intra or extracellulary to the affected cells. The protein aggregates may comprise tau, TARDBP and FUS.

Prion diseases are a class of infectious diseases transmitted by prion proteins, such as major protein (PrP), and include Creutzfeldt-Jakob disease, new variant Creutzfeldt-Jakob disease (nvCJD), Gerstmann-Sträussler-Scheinker syndrome, fatal familial insomnia and kuru. They are caused by mis-folded prion proteins that form into aggregates and lead to the loss of brain cells. The disease is transmitted when healthy people or animals consume tissue from those carrying the disease.

Type II diabetes is a metabolic disorder that is characterized by high blood glucose in the context of insulin resistance and relative insulin deficiency. It is associated with a number of complications, including increased risk of cardiovascular disease, an increased requirement for lower limb amputation, blindness, kidney failure and dementia. Type II diabetes may be associated with the formation of amyloid protein aggregates composed of islet amyloid polypeptide (IAPP) in the pancreas, which leads to the loss of pancreatic cells and a decrease in the level of insulin production.

Inclusion body myopathy is an inflammatory muscle disease, characterized by slowly progressive weakness and wasting of both distal and proximal muscles, most apparent in the muscles of the arms and legs. It may be associated with the formation of protein aggregates in muscle fibres which comprise of amyloid-beta, phosphorylated tau protein, and at least 20 other proteins that are also accumulated in the brain of AD patients.

### Diagnosing

In a further aspect the present invention relates to a method for diagnosing a disease associated with protein aggregation. The present invention also provides a method for determining if a subject is at risk of developing a disease associated with protein aggregation.

Each of the above methods comprises the step of determining the level of effective RNA in a sample from a subject.

The level of effective RNA in the sample refers to the level of RNA which is capable of sustaining and/or promoting the folding of proteins (i.e. preventing and/or reducing the level of protein aggregates).

The level of effective RNA may be reduced in the sample because of a lower amount/total quantity of RNA, a loss of RNA integrity or because of the deposition or removal of specific post-transcriptional modifications (e.g. methylation) on the RNA.

A loss of integrity refers to a decrease in the total quantity of RNA which has a native (unaltered) structure in the sample. The structure of RNA may be altered, for example, by a change (e.g. a reduction) in the levels of divalent ions (e.g. Mg²⁺, Ca²⁺, or Zn²⁺) within the cell, or a reduction in the level of divalent ions which are associated with RNA molecules.

A reduction or decrease in the level of effective RNA may be caused by RNA degradation.

The sample may be a serum, plasma, cerebrospinal fluid sample or a tissue sample such as a brain, pancreatic or muscle sample.

RNA may be isolated from human blood, white blood cells, cerebrospinal fluid (CSF), plasma, serum, or biopsies (e.g. brain). Kits and reagents, e.g. Trizol LS (Life Technologies) or QIAamp RNA Blood Mini Kit (Qiagen), are commercially available to isolate RNA from these sources.

The amount of the RNA may be determined by quantitative PCR (q-PCR), Northern Blot or specific complementary probes (e.g. molecular beacons that only emit fluorescence when bound to their targets). Integrity of the RNA may be determined by Northern Blot, and/or complementary probes. Methylated RNA could be identified by bisulfite sequencing, oxidised RNA (8-OHG) by immunoprecipitation with specific 8-OHG antibodies (e.g Neurochem Res (2006) 31:705-710) followed by q-PCR or antisense molecular probes (e.g LNA). Other modifications could be determined by mass spectrometry.

The level of effective RNA in the sample is determined by comparison to a control.

Reference to a “control” broadly includes data that the skilled person would use to facilitate the accurate interpretation of technical data. As such “control level” is interchangable with “reference level”. In an illustrative example, the level effective of RNA is compared to the respective level of effective RNA in one or more cohorts (populations/groups) of control subjects selected from a cohort wherein the subjects have been diagnosed with a condition which is associated with protein aggregation at a particular site and a cohort wherein the subjects have been predetermined not to have a condition which is associated with protein aggregation at a particular site.

Where the control is derived from a cohort which has been predetermined not to have a condition which is associated with protein aggregation at a particular site, the following determinations in the test sample may be indicative of a disease associated with protein aggregation: an amount of RNA which is 2, 3, 5, 10, 100 or 1000-fold less than the control level, an increased level of RNA degradation (i.e. 2, 3, 5, 10, 100 or 1000-fold greater) or a lower level of RNA integrity (i.e. 2, 3, 5, 10, 100 or 1000-fold less) compared to the control sample, a difference in the level of a specific post-transcriptional modification or a combination of post-transcriptional modifications of the RNA compared to the control sample.

Where the control is derived from a cohort in which the subjects have been diagnosed with a condition which is associated with protein aggregation at a particular site, the following determinations in the test sample may be indicative of a disease associated with protein aggregation: an amount of RNA which is within 1, 2, 5, 10 or 20% of the control level, a level of RNA degradation or RNA integrity in the test sample which is similar to the control sample (i.e. within 1, 2, 5, 10 or 20% of the control level), a similarity in the level of a specific post-transcriptional modification or a combination of post-transcriptional modifications of the RNA compared to the control sample.

The control or reference levels for the detection of a given level or state (i.e. integrity or modification state) of effective RNA at a particular site may be stored in a database and used in order to interpret the results of the method as performed on the subject.

### Animal Model

In a further aspect the present invention relates to an animal model for a disease associated with protein aggregation, in which animal protein aggregation is induced by the removal of RNA in a cell in the animal.

The RNA may be ribosomal RNA.

The term “RNA removal”, as defined herein, means to reduce the total quantity of intact RNA molecules or reduce the total quantity of RNA which has a native (unaltered) structure in a cell in the animal.

RNA removal may be or involve RNA degradation.

The term “degradation” is used herein in its conventional sense to relate to the destruction of the RNA within a cell or cell lysate. Destruction of the RNA may be achieved by the disruption of the primary structure of an RNA molecule via the cleavage of the phosphodiester bonds between adjacent nucleotides.

Degradation of RNA may be achieved through the use of ribonucleases. Ribonucleases (RNase) are a type of nuclease which catalyse the degradation of RNA molecules into smaller components. RNases can degrade either single-stranded or double-stranded RNA, depending on the specific enzyme, and are generally defined by their mechanism of action as being divided into endoribonucleases and exoribonucleases.

An exoribonucease is an enzyme which degrades RNA by removing terminal nucleotides from either the 5′ end or the 3′ end of an RNA molecule. Enzymes that remove nucleotides from the 5′ end are termed 5′-3′ exoribonucleases and enzymes that remove nucleotides from the 3′ end are termed 3′-5′ exoribonucleases. Examples of exoribonucleases include, but are not limited to, RNase R, RNase II, Rrp44, RNase D, RNase T, PM/Scl-100, Oligoribonuclease, RNase BN, PNPase, PM/Scl-75, RNase PH, RRP4, Exoribonuclease I and Exoribonuclease II.

An endoribonuclease is an enzyme which cleaves the phosphodiester bond between adjacent nucleotides in an RNA molecule, wherein neither of the nucleotides is the terminal nucleotide of the RNA molecule. Examples of endoribonucleases include, but are not limited to, RNase III, RNase A, RNase T1, RNase 1f, RNase H, RNase V1 and also complexes of proteins with RNA like RNase P and the RNA-induced silencing complex (RISC).

The RNA removal may involve the administration of, or increasing the expression or activity of, a ribonuclease.

The ribonuclease may be RNase A, RNase T1 and/or RNase 1f.

RNA removal may alternatively involve altering the structure of the RNA. This reduces the ability of the RNA to solubilise proteins. The structure of RNA may be altered by changing the concentration of (e.g. by removing) divalent ions, such as Mg²⁺, Ca²⁺, or Zn²⁺, which are important for RNA folding. Agents such as EDTA can be used to remove divalent ions and have previously been used to dissociate ribosomes.

The removal of RNA may be achieved by administration of antisense RNA or short-inhibiting RNA (siRNA) to the animal.

The removal of RNA may be achieved by inducing a reduction in RNA expression in a cell in the animal.

The animal may be a mammal. For example, the animal may be a primate, mouse, rat, guinea pig or rabbit.

The protein which is aggregated following the reduction in the effective amount of RNA may be selected from the following: amyloid-β, MAPT, SCNA, TARDBP, FUS, HTT, PrP, neurofilament (NF-H) and alpha-synuclein.

The removal of RNA may cause the simultaneous aggregation of the plurality of proteins.

The plurality of proteins may aggregate together, forming an aggregate comprised of a plurality of protein types.

“Plurality” indicates that RNA removal causes aggregation of at least 2 proteins, for example, 3, 4, 5, 6, 7, 8 or 9 proteins.

The plurality may comprise 2, 3, 4, 5, 6, 7, 8 or all or the following proteins: amyloid-β, MAPT, SCNA, TARDBP, FUS, HTT, PrP, neurofilament (NF-H) and alpha-synuclein.

### Ribonucleoside Vanadyl

The present invention further relates to the use of ribonucleoside vanadyl to initiate the aggregation of a plurality of proteins in a cell or cell lysate.

Vanadyl ribonucleoside is a low molecular weight inhibitor of ribonucleases. It is used as a transition state analogue inhibitor of RNAses during RNA purification and manipulation. The present inventors have surprisingly shown that, when added to a cell lysate, it causes substantial protein aggregation.

The present invention also provides an animal model for a disease associated with protein aggregation, in which animal, protein aggregation is induced using ribonucleoside vanadyl and/or divalent ions.

The animal model may be an animal as defined herein.

### In Vitro Refolding

The present inventors have shown that proteins refolded with RNA in vitro become functional, as exemplified by active ATP hydrolysis.

As such, in one aspect the present invention relates to an in vitro method for promoting the folding of a protein which comprises the step of contacting an unfolded or partially folded protein with RNA or DNA (e.g. genomic DNA) in order to promote folding.

In one aspect, the present invention relates to an in vitro method for promoting the folding of a protein which comprises the step of contacting an unfolded or partially folded protein with nucleic acid, for example RNA, DNA (e.g. genomic DNA) or LNA, or with sodium orthovanadate, or a derivative, structural mimic or modified version thereof, in order to promote folding.

In another aspect, the present invention relates to a method for promoting the folding of a protein which comprises the step of contacting an unfolded or partially folded protein with nucleic acid, for example RNA, DNA (e.g. genomic DNA) or LNA, or with sodium orthovanadate or a derivative, structural mimic or modified version thereof, in order to promote folding, wherein said method is carried out in an isolated cell. In one embodiment the isolated cell is a bacterial cell (e.g. an E. coli cell). In one embodiment the isolated cell is a eukaryotic cell (e.g. a yeast cell or an animal cell that has been isolated from an animal such as a mammal or a zebrafish); in one embodiment the isolated cell is a mammalian cell.

The terms ‘unfolded’ and ‘partially folded’ refer to proteins which do not have a native structure (i.e. a loss of RNA structure integrity). In particular the terms refer to a peptide which does not have the required secondary and tertiary structures to produce a functional protein.

‘To promote folding’ means that the protein forms the required secondary and tertiary structures and is therefore functional. Productive folding of a protein can be determined using methods known in the art, which will vary depend on the specific function of the protein to be folded.

The present invention further provides a use of RNA or DNA (e.g. genomic DNA) to promote the in vitro folding of an unfolded or partially unfolding protein, for example a therapeutic protein or biological reagent, such as an enzyme, antibody, protein ligand, receptor, structural protein or cofactor.

The RNA or DNA (e.g. genomic DNA) may comprise a G-quadruple structure.

The method or use described above may be used to provide a functionally folded transmembrane protein.

The term ‘transmembrane protein’ refers to membrane protein spanning the entirety of the membrane. Examples of transmembrane proteins include, but are not limited to, α-helical transmembrane proteins, for example G-protein coupled receptors.

These proteins are usually very difficult to produce as they tend to aggregate, a fact reflected in their sparse representation in the protein data bank (PDB). However, transmembrane proteins represent one of the most druggable and sought after targets. Screening for drugs against these targets is frequently achieved through cell-based assays where, for example, an ion channel is ectopically expressed. However, the cellular nature of these assays makes them expensive and cumbersome. The manufacturing of correctly folded transmembrane proteins would be a significant advantage for drug screening assays but would also have potential applications outside this area.

The method or use described above may be used to provide a functionally folded therapeutic protein or biological reagent, such as an enzyme, antibody, protein ligand, receptor, structural protein or cofactor.

The invention will now be further described by way of Examples, which are meant to serve to assist one of ordinary skill in the art in carrying out the invention and are not intended in any way to limit the scope of the invention.

### EXAMPLES

### Example 1—RNase-Mediated Precipitation of Proteins

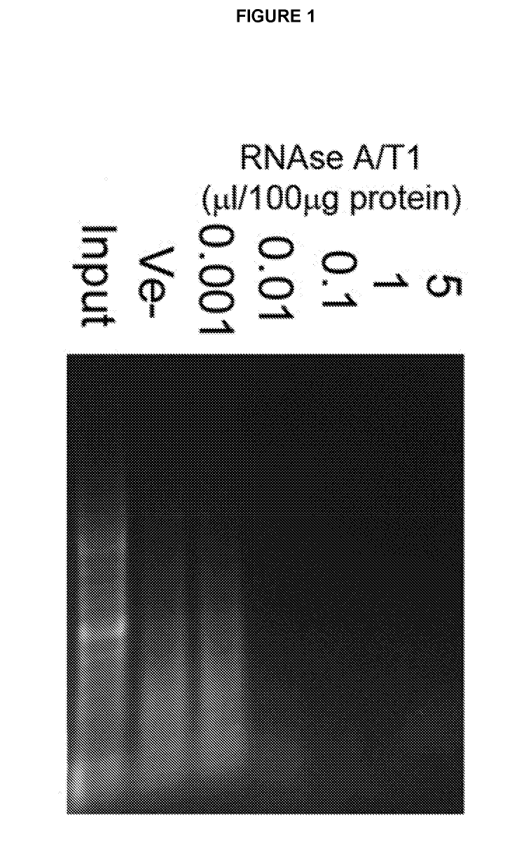

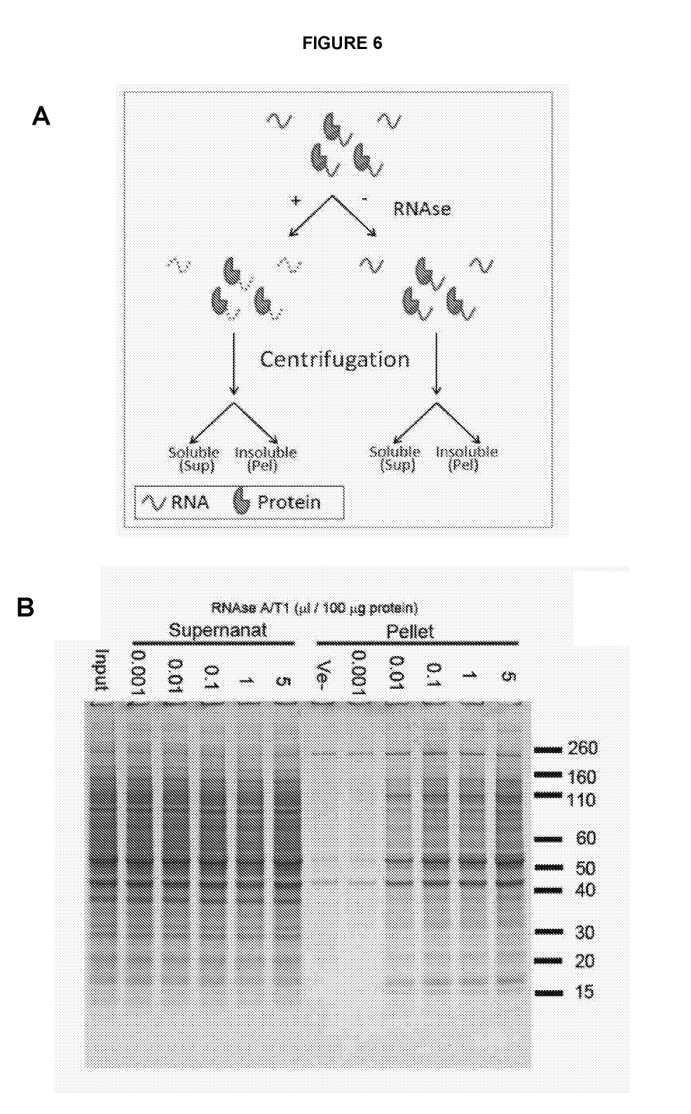

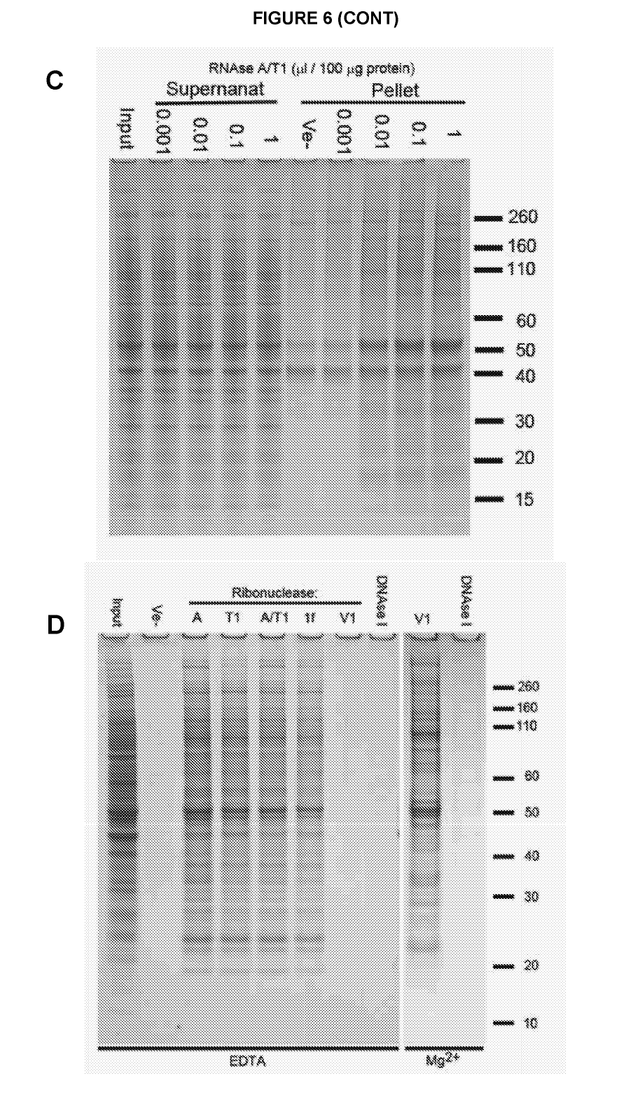

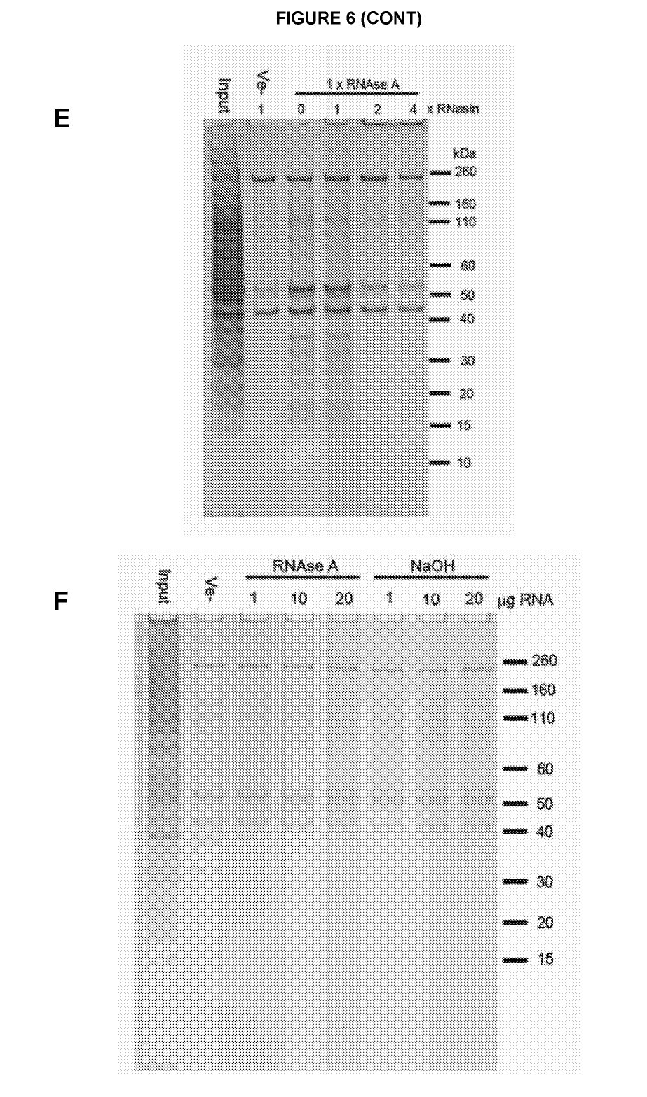

Lysates prepared from human neurons and mouse brain cortex were treated with RNAse A and T1 and the precipitated proteins were analysed. RNase treatment caused a concentration-dependent precipitation of several proteins from both human neurons and mouse brain (FIGS. 6b and c). Comparing the protein profiles of the same amount of protein from the input and the RNase-precipitated samples showed that many of the precipitated proteins were enriched compared to input. No detectable difference was observed between the input and the supernatant. In addition, analysis of RNA upon completion of RNase-mediated digestion indicated that the amount and size distribution of recovered RNA decreased with increasing RNase concentration (FIG. 1).

### Example 2—Determining the Impact of the Source of RNase

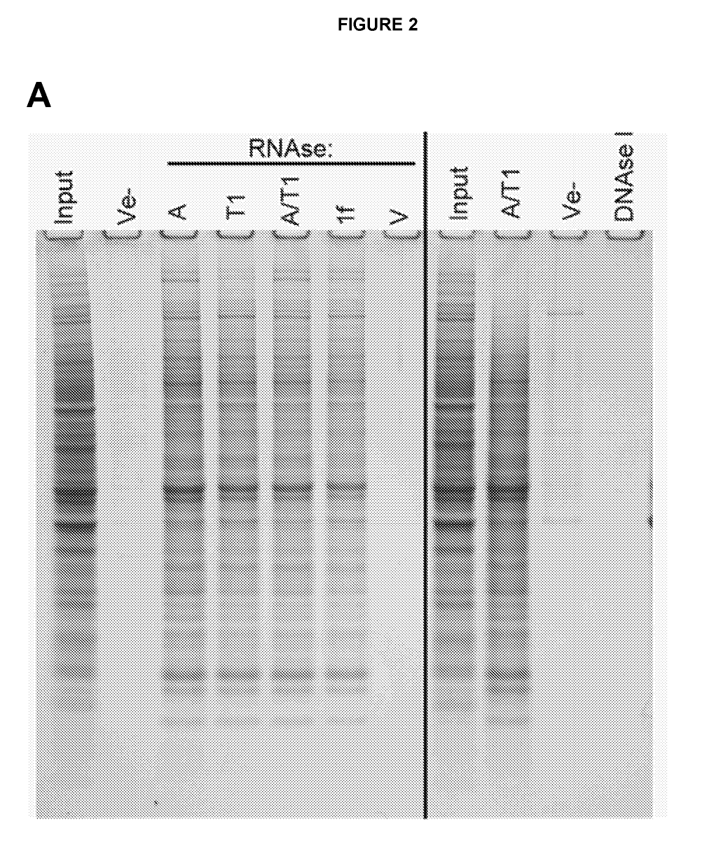

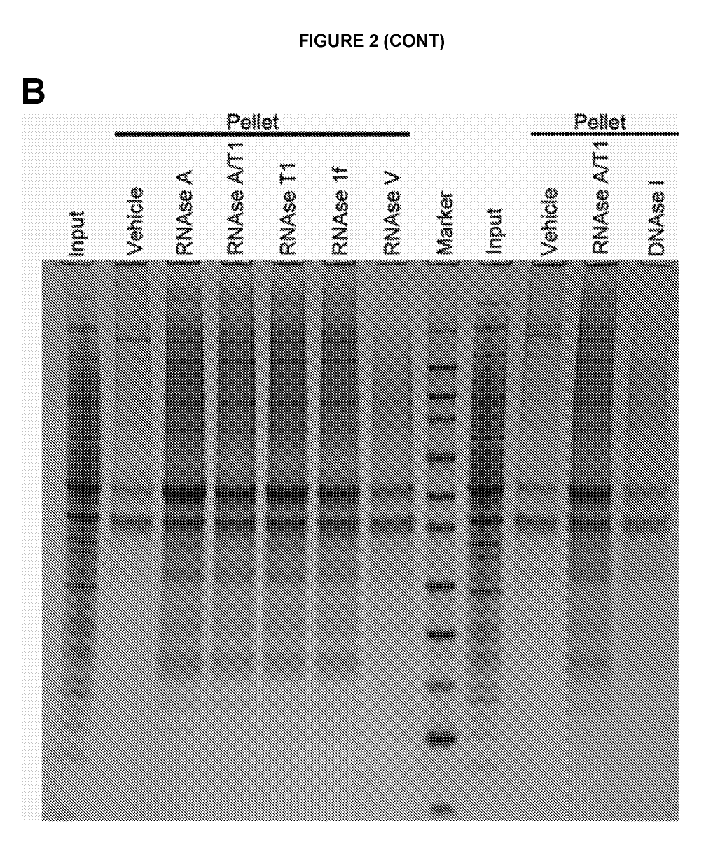

The aggregation effect was tested with various different RNases. It was shown that the source of single-stranded specific RNase was not important for the overall efficiency of protein precipitation and each of RNase A, RNase T1 and RNase 1f showed similar efficiency to the RNase/T1 mixture (FIGS. 2 and 6d). However, digestion with RNase V1, which is specific for double-stranded RNA, or DNase I failed to cause protein precipitation (FIG. 2). RNAse V1 only caused protein precipitation if EDTA was omitted from the buffer and replaced with Mg²⁺ (FIG. 6d), consistent with the requirement of RNAse V1 for divalent ions. However, DNAse I failed to cause protein precipitation under both conditions (FIG. 6d). No proteins above background were precipitated when ribonuclease inhibitors were added to the lysate together with RNAse A (FIG. 6e). Furthermore, to ensure that the ribonuclease degradation products were not responsible for protein precipitation, isolated RNA was digested with immobilised RNAse A or by alkaline hydrolysis and the digest then added (without RNAse A) to cell lysate. No proteins above background were precipitated by the addition of enzymatically or chemically degraded RNA (FIG. 6f). Taken together, these experiments show that the solubility of the precipitated proteins depends on intact cellular RNA.

### Example 3—Identification of the Proteins Precipitated by RNA Removal

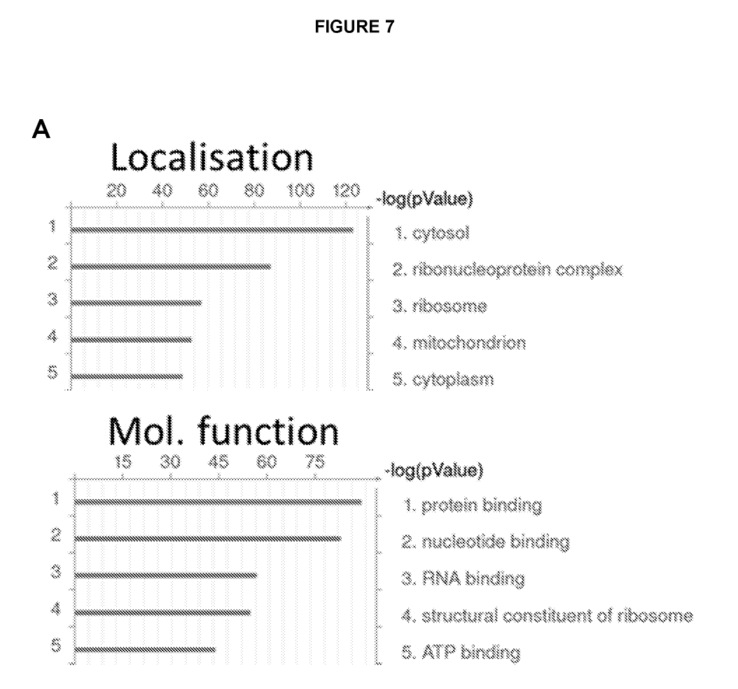

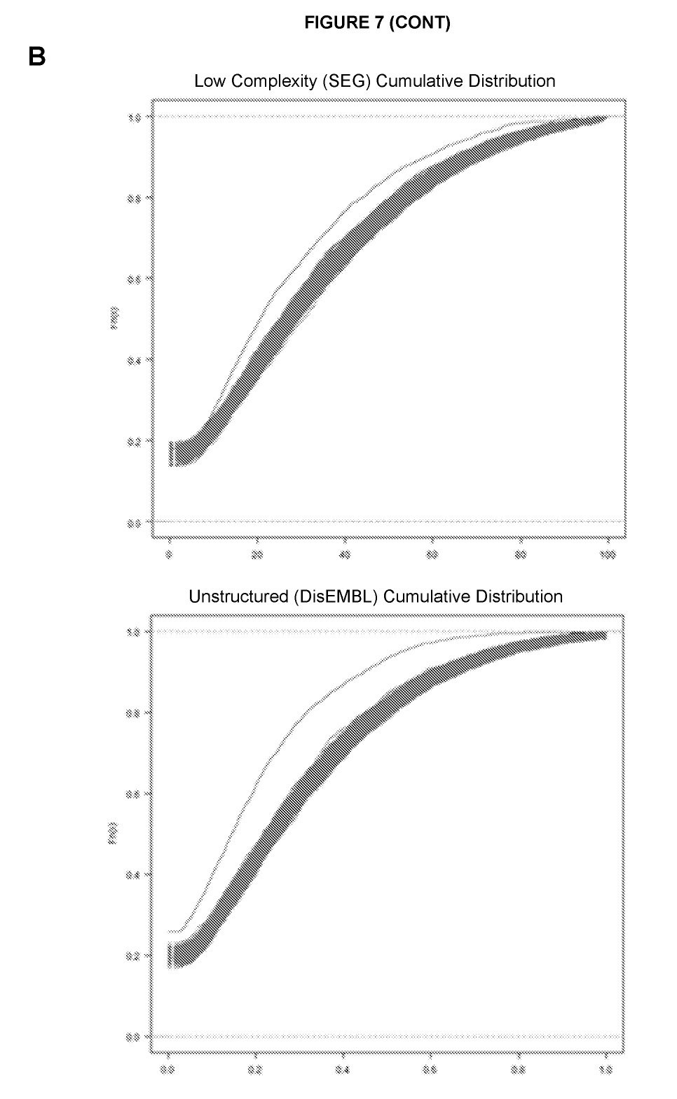

To identify the proteins which are precipitated by RNA removal in human neurons, precipitated proteins were separated from two independent experiments by SDS-PAGE followed by tandem mass spectrometry (LC-MS/MS) analysis. More than 1600 proteins were identified which were common to both samples, representing an overlap of more than 75% (FIG. 7a). Gene ontology analysis of the data-set indicates firstly; an over-representation of cytosolic proteins and ribonucleoprotein complexes, and secondly, a significant over-representation of proteins involved in protein-protein interactions (60%, 982), nucleotide binding (26%, 438), RNA binding (13%, 214), and structural ribosomal proteins (95) (FIG. 7b). There are no obvious sequence or structural similarities between the precipitated proteins. Unstructured, low-complexity regions in several RNA-binding proteins have previously been suggested to mediate protein aggregation and to be required for recruitment to stress granules (Kato et al (2012) Cell 149, 753-767). Although several of the proteins identified in these studies also aggregate after RNA removal (see Table 1 below), both unstructured (US) and low complexity (LC) regions are significantly under-represented in the data-set (FIG. 2c, p=2.9×10-18 for US and p=7.2×10-5 for LC), indicating that the majority of the proteins are globular, a finding consistent with the notion that globular proteins harbour more aggregate-prone regions.

TABLE 1
Proteins which are found aggregated by Kato et al.,
and are also aggregated by RNA removal.

EIF2S3
CALR
CNOT7
DDX1
DDX39B
DDX3X
DDX5
DDX6
DHX36
DHX9
EDC3
EDC4
EIF3A
EIF4A1
EIF4G1
ELAVL1
ELAVL3
FXR1
GNB2L1
HNRNPA0
HNRNPA1
HNRNPC
HNRNPD
HNRNPH1
HNRNPK
HNRNPM
HNRNPU
HSPA8
HTT
IGF2BP1
ILF2
IPO8
KHDRBS1
KLC1
MATR3
MSI1
NCL
NONO
NPM1
NXF1
PCBP2
PLEC
PSPC1
PTK2
PUM1
PURA
PURB
RPL3
SFPQ
SLC25A10
TARDBP
UPF1

### Example 4—Determination of the Precipitation of Proteins Associated with Neurodegenerative Diseases

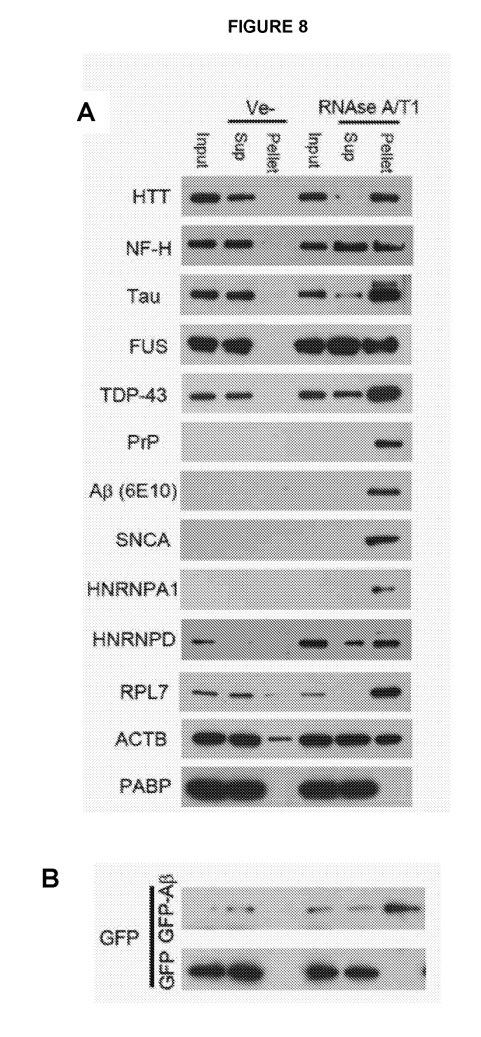

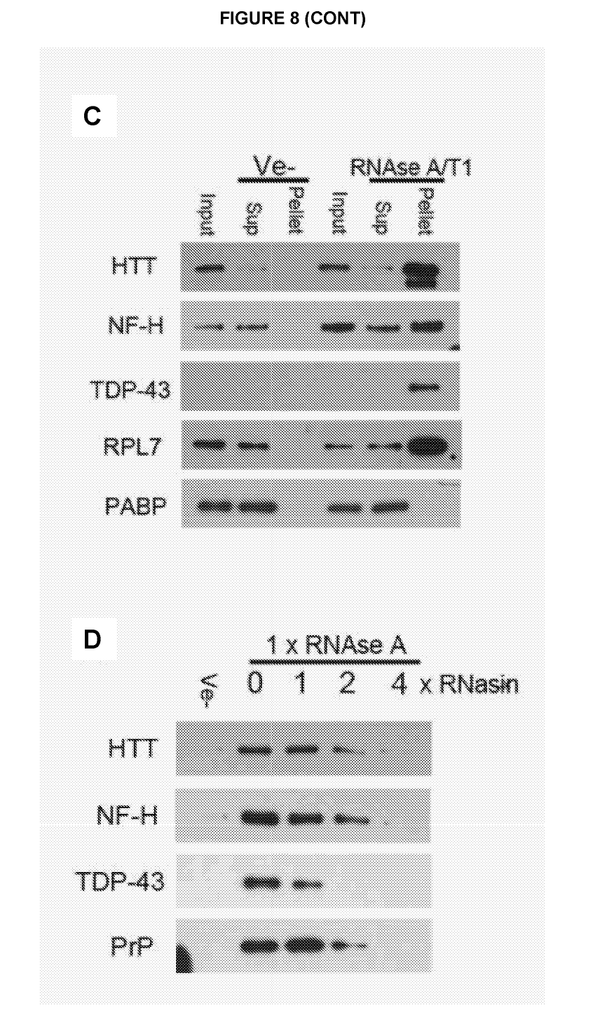

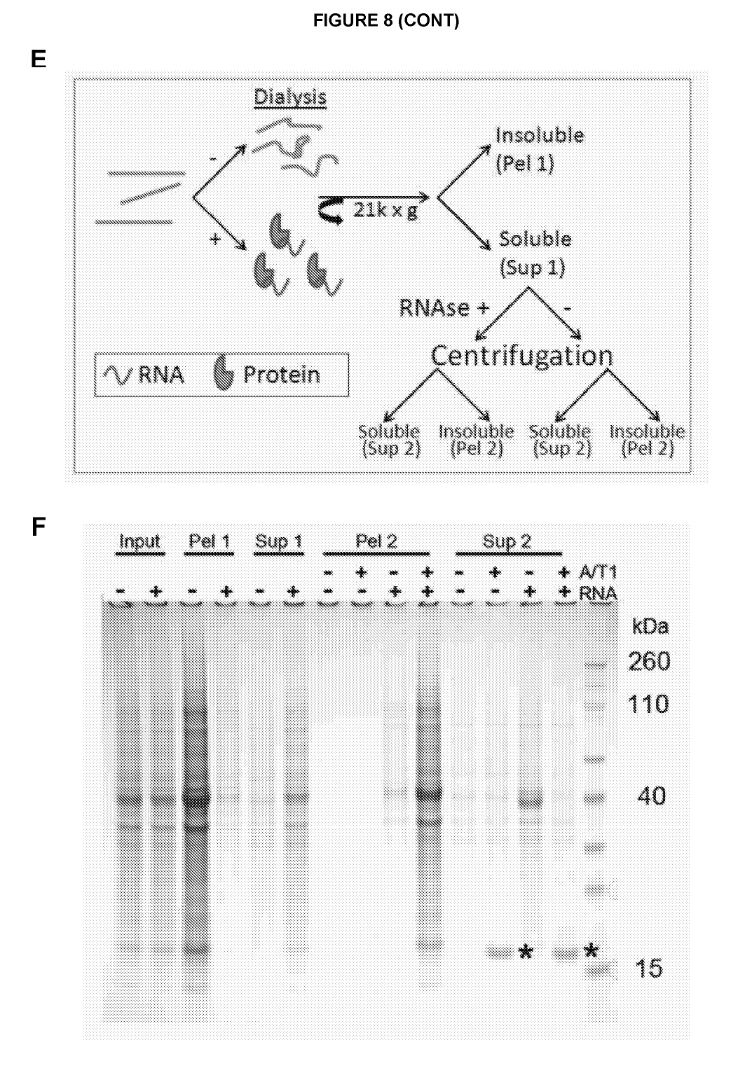

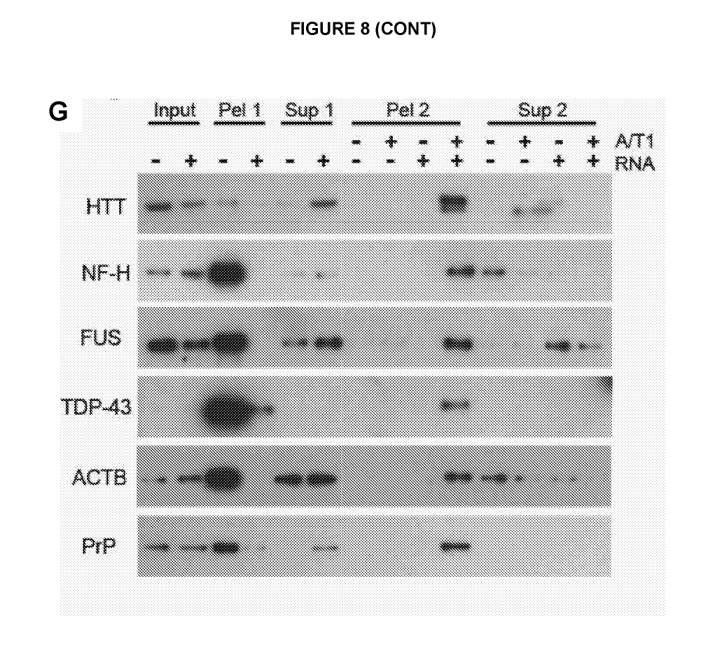

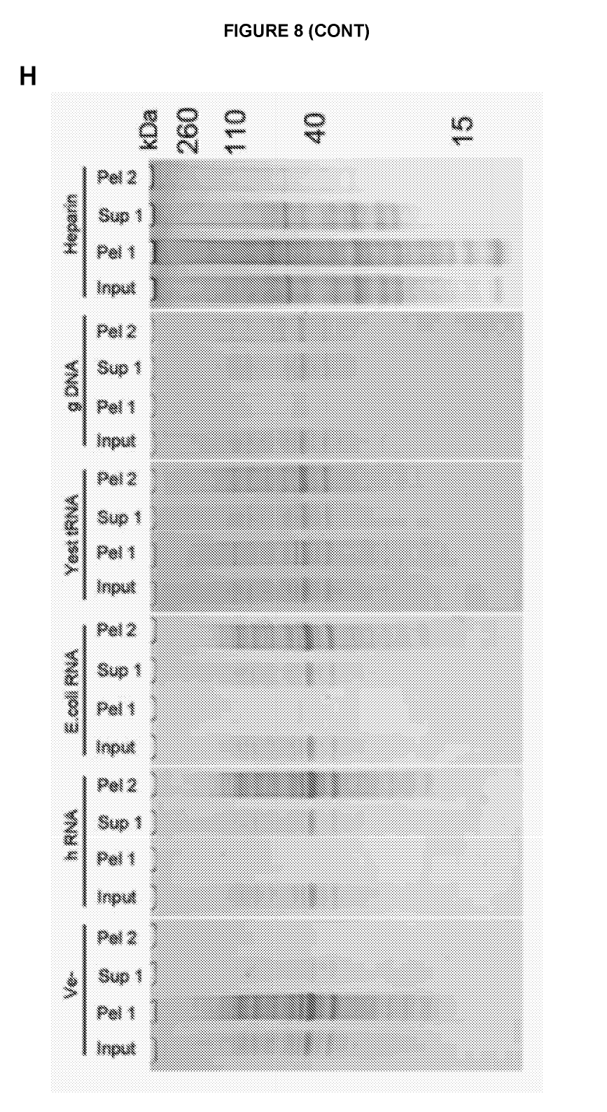

Since several aggregate-prone proteins associated with neurodegenerative disease were among the list of precipitated proteins, including huntingtin (HTT), TDP-43, hnRNPA2B1 and hnRNPA1, western blotting was used to investigate the solubility of other aggregation-prone proteins associated with these disorders. All proteins investigated, including amyloid-β, tau (MAPT), α-synuclein (SNCA), TARDBP (TDP-43), FUS, HTT, the prion protein (PrP), HNRNPA1, actin (found aggregated in Hirano bodies in several neurodegenerative diseases), and neurofilament heavy chain (NF-H) were selectively precipitated upon RNAse A/T1 treatment of human neuronal lysates (FIG. 8a). For example PrP, alpha-synuclein, HTT and amyloid-β were all below detection in the soluble fraction after RNase treatment (FIG. 8a).

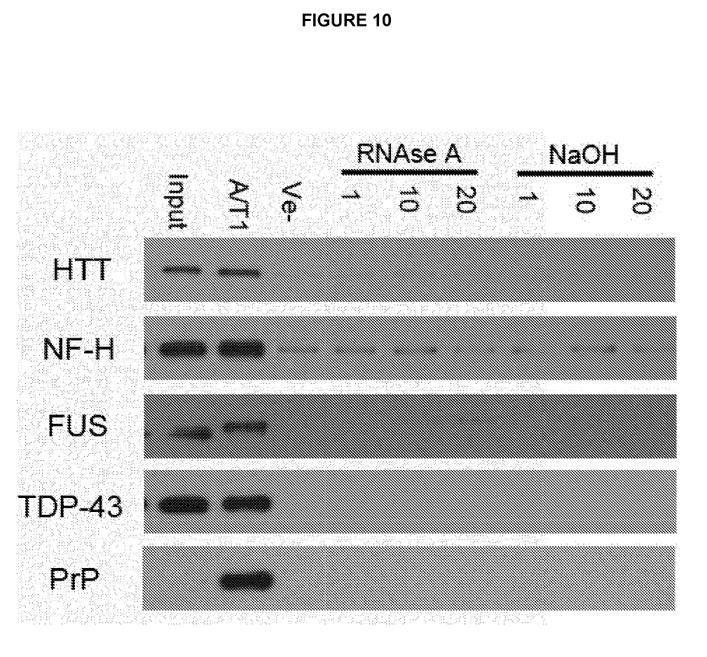

We also detected an approximately 40 kDa Aβ immunoreactive band in the pellet of RNAse-treated lysate from human neurons (FIG. 8a). Since the molecular weight of this band is larger than expected (Aβ monomers migrate at ˜4 kDa) the aggregation of Aβ in cell lysates prepared from HEK293 cells expressing Aβ fused to GFP was also examined. Similar to endogenous Aβ (FIG. 8a), GFP-tagged Aβ only aggregated upon removal of RNA (FIG. 8b). No aggregation was observed for GFP itself (FIG. 8b). The solubility of two proteins not directly related to neurodegeneration but precipitated by RNAse-treatment was then investigated; the ribosomal protein RPL7 and the heterogeneous nuclear ribonucleoprotein D (HNRNPD) as well as the poly A binding protein (PABP, an abundant protein but not identified by mass spectrometry) as a control. Of these, RLP7 and HNRNPD were precipitated by RNA digestion while the solubility of PABP was unaffected (FIG. 8a). Similar results were obtained using tissue lysate prepared from mouse cortex (FIG. 8c). Inhibiting the added RNAse activity with RNasin abolished the precipitation of HTT, NF-H, TDP-43, and PrP (FIG. 8d), confirming the inhibition observed on the global protein profile (see FIG. 6e). Similarly, addition of in vitro degraded RNA failed to cause precipitation of HTT, NF-H, FUS, TDP-43, and PrP (FIG. 10). Together, these experiments show that many proteins associated with neurodegenerative disorders are dependent on RNA for their solubility in cell-free lysates.

### Example 5—Characterisation of Proteins Precipitated Following Treatment with Either RNase A or RNase T1

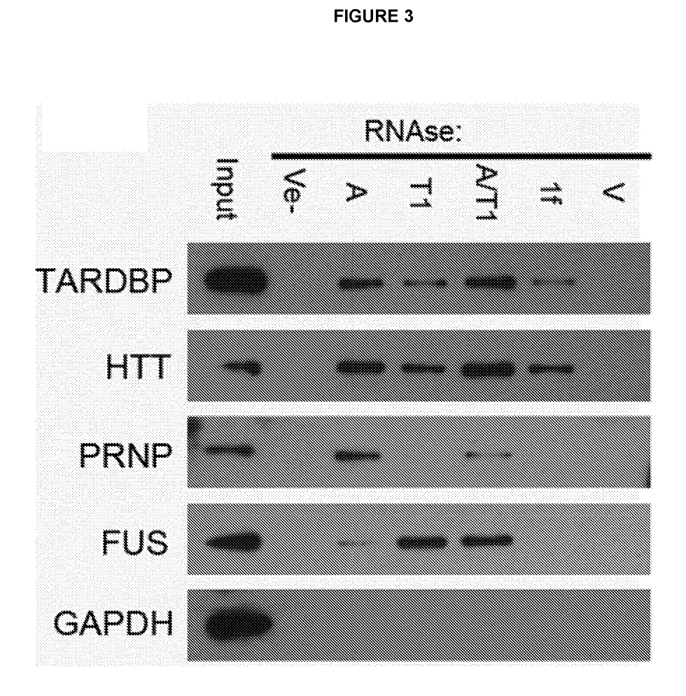

The aggregation of proteins after digestion with either RNase A or T1 was analysed. Both of these RNases cleave single-stranded RNA, but with different specificities, as RNase A cleaves after C and U whilst RNase T1 cleaves after G. TARDBP, HTT and PrP were most efficiently precipitated by RNase A whilst FUS was most efficiently precipitated by RNase T1 (FIG. 3).

### Example 6—RNA-Mediated Protein Re-Folding

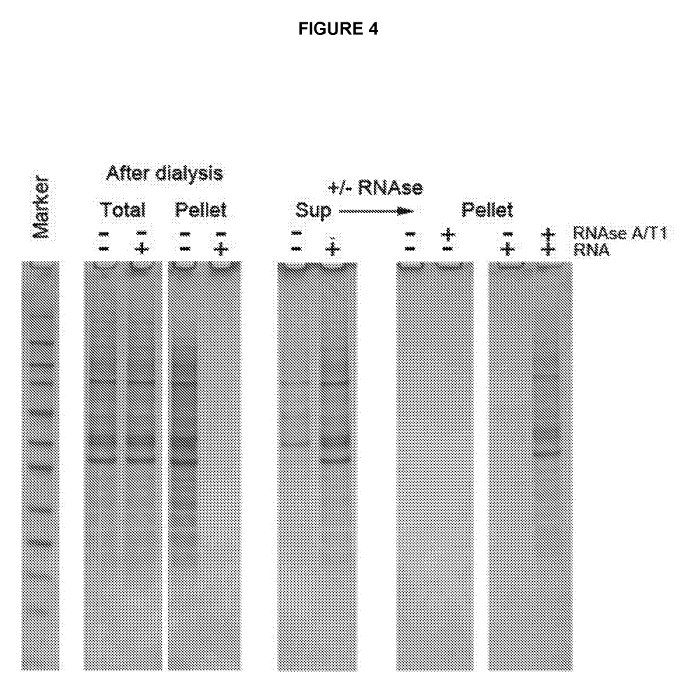

In order to investigate RNA-mediated protein refolding, aggregated protein formed following RNA removal was denatured and then treated with RNA to induce re-folding (Figure Be). These data indicate that proteins that aggregate following the removal of RNA can be efficiently re-folded in vitro, but only in the presence of RNA (FIGS. 4 and 8f and g). However, the RNA is not only required for their initial folding but also for their continuing solubility since degradation of RNA, after re-folding, reversed the process and caused the proteins to re-aggregate (FIGS. 4, 8f and 8g).

### Example 7—Investigating the Capacity of Other Polyanions to Re-Fold Proteins

It was investigated whether the capacity to re-fold the proteins was specific to human RNA or a common feature of other polyanions, including total E. coli RNA, yeast tRNA, human genomic DNA (gDNA) and heparin. E. coli RNA efficiently prevented protein aggregation while neither yeast tRNA nor heparin could substitute for the solubilising capacity of total human RNA (FIG. 8h). Surprisingly, the addition of human genomic DNA was almost as efficient as total human RNA in solubilising the proteins (FIG. 8h). However, only the proteins re-folded in the presence of human RNA or E. coli RNA were re-aggregated upon RNAse treatment (FIG. 8h, Pel 2). The ability of genomic DNA to facilitate re-folding is unexpected, as the experiments described above clearly show that the proteins in cell lysate are dependent on RNA and not DNA for their aggregate-free state (FIG. 6d).

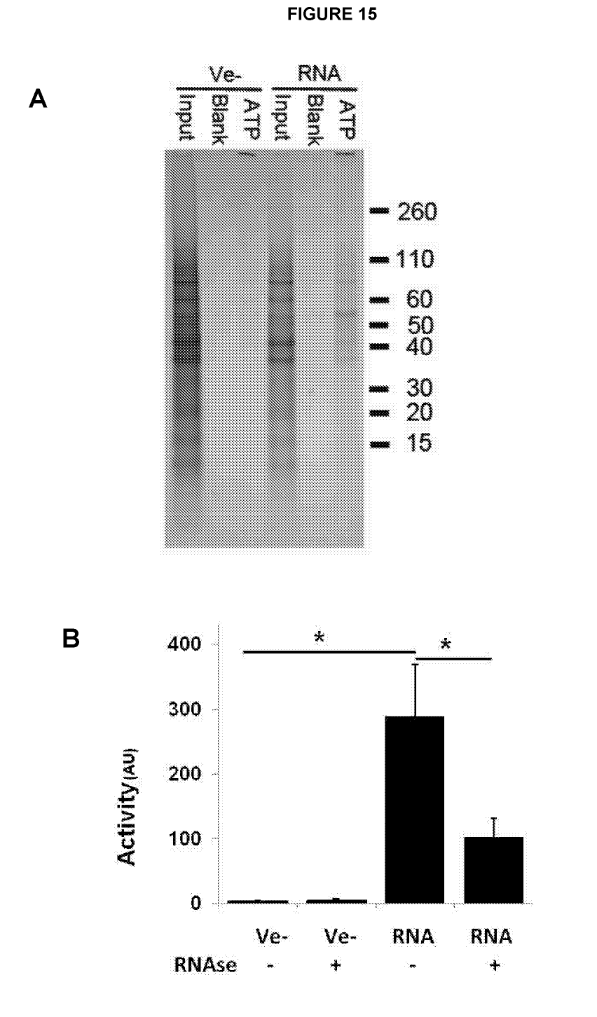

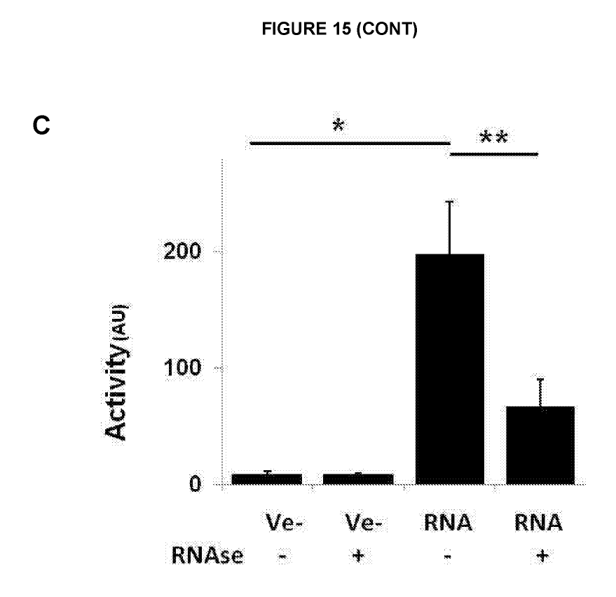

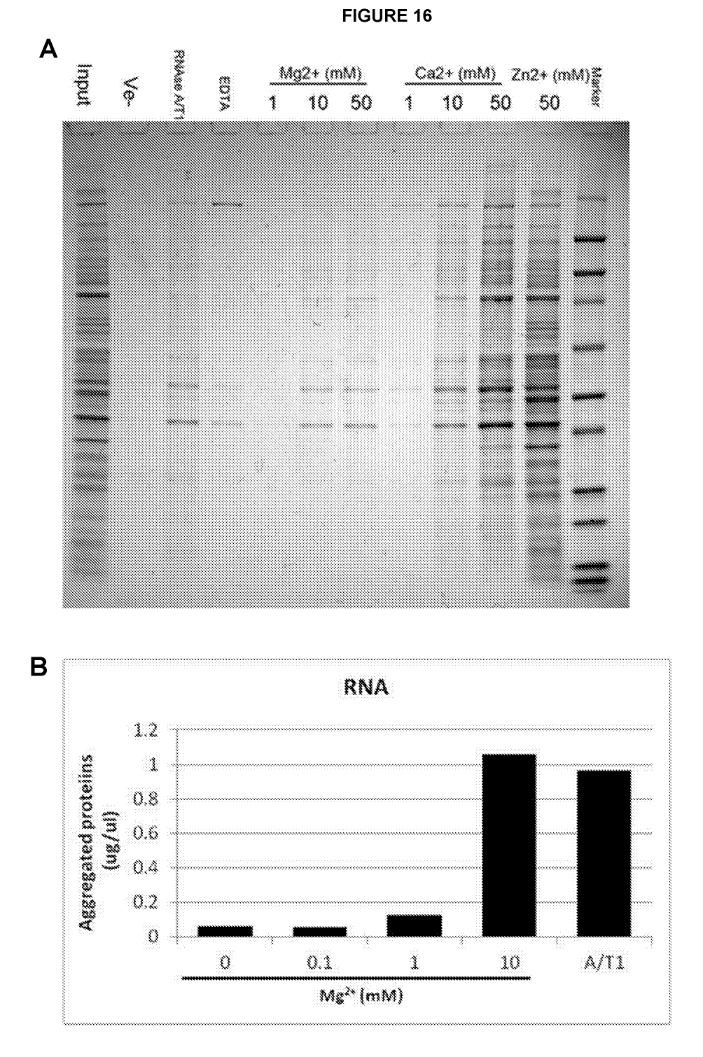

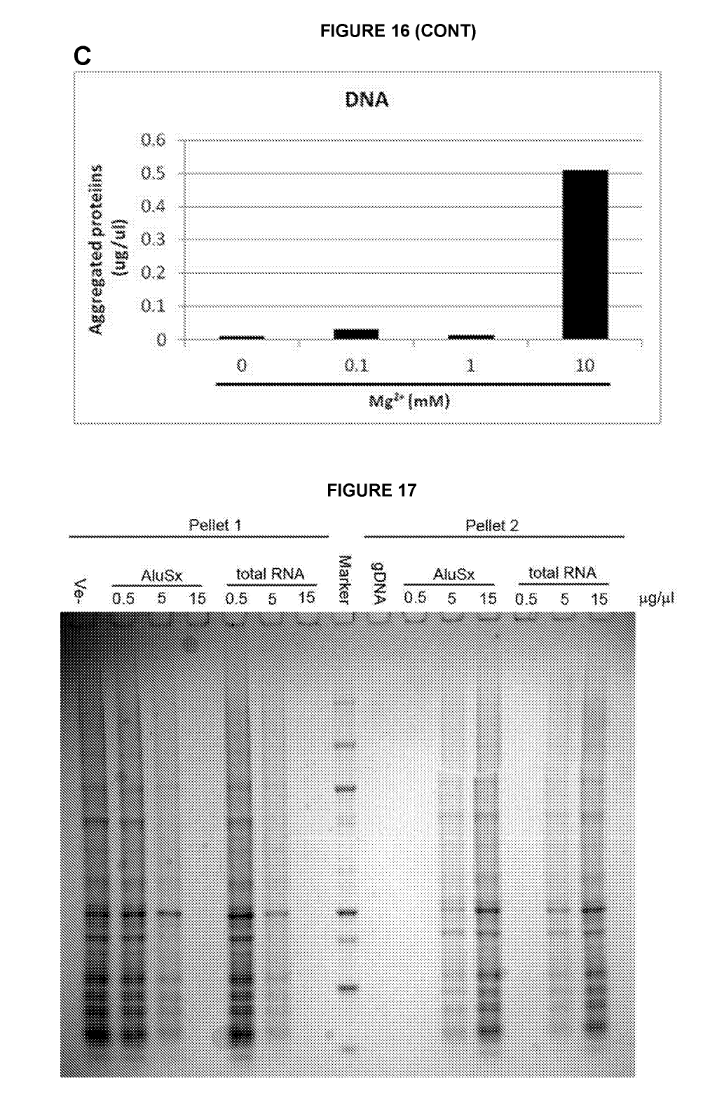

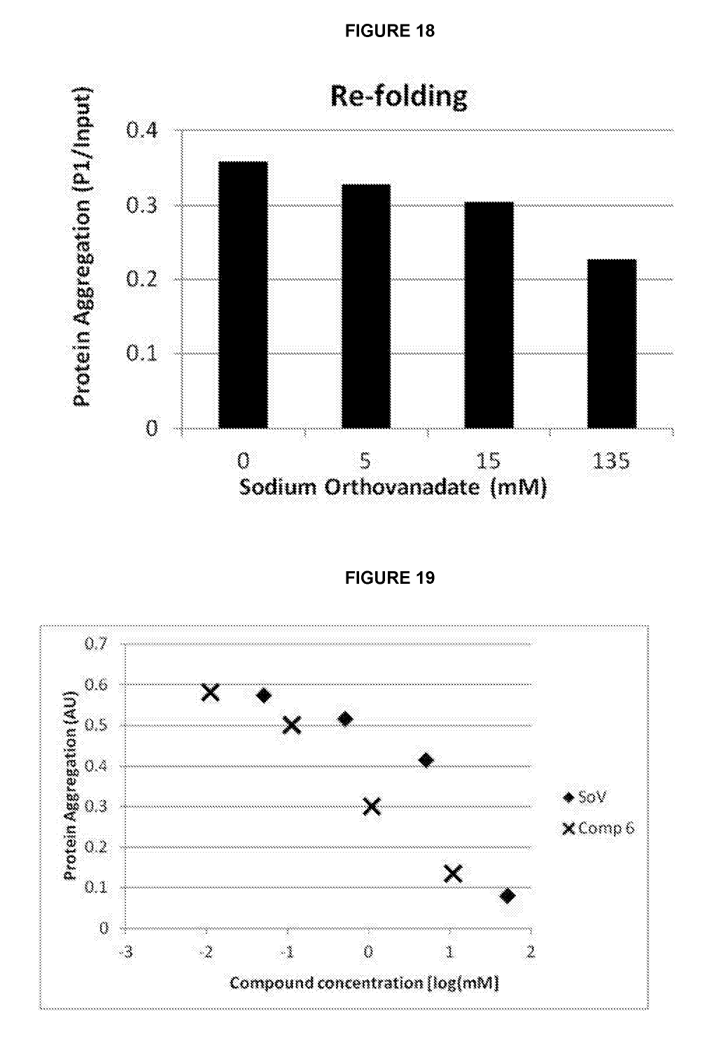

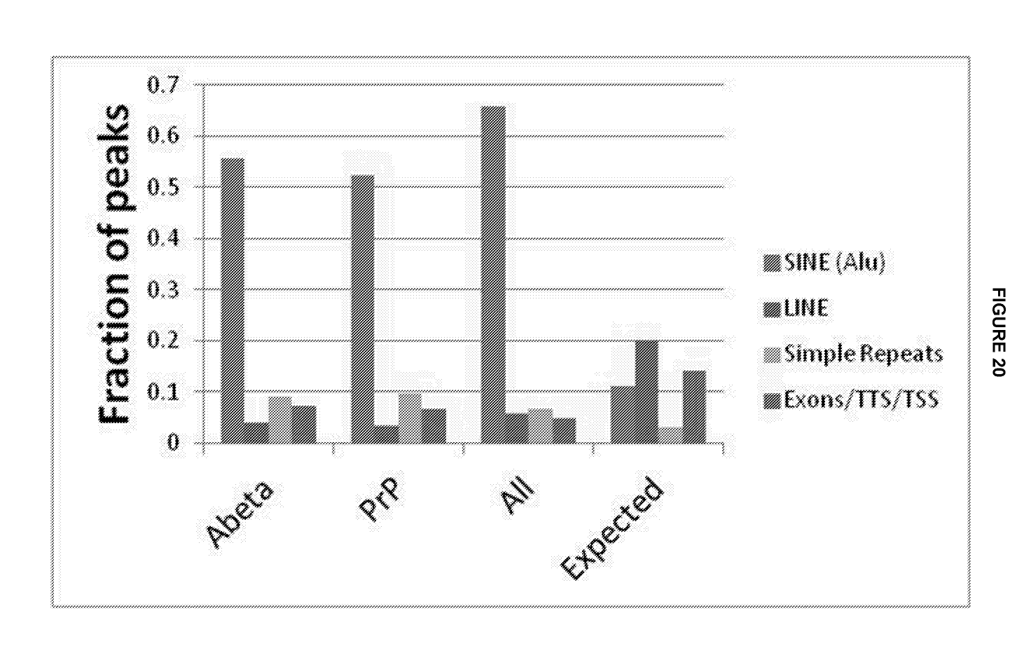

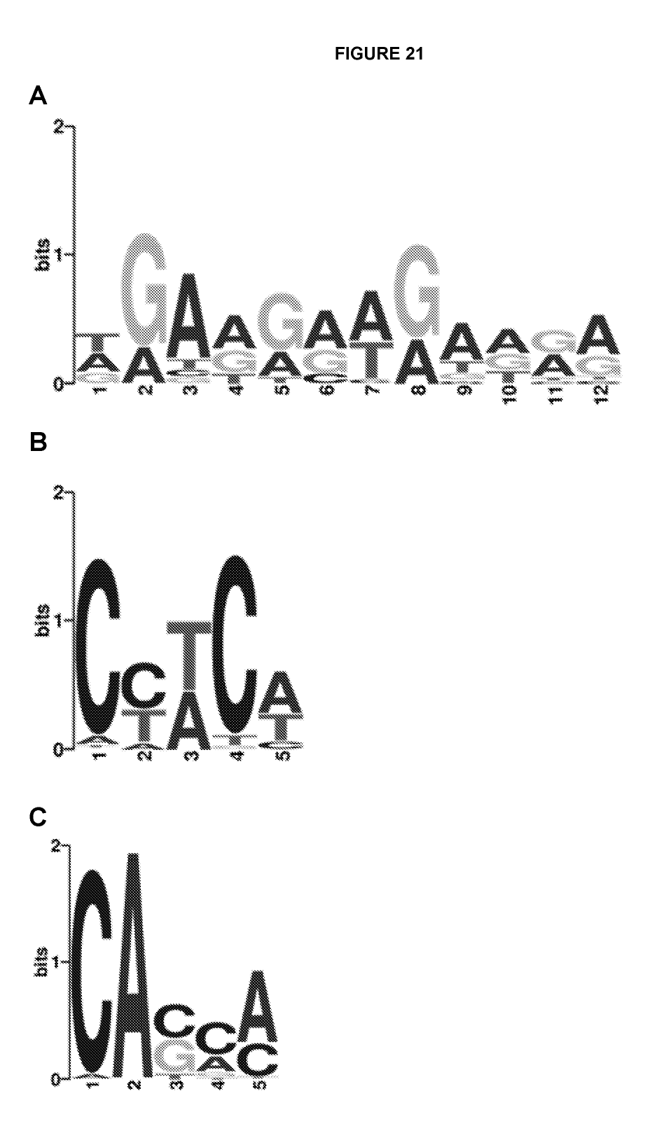

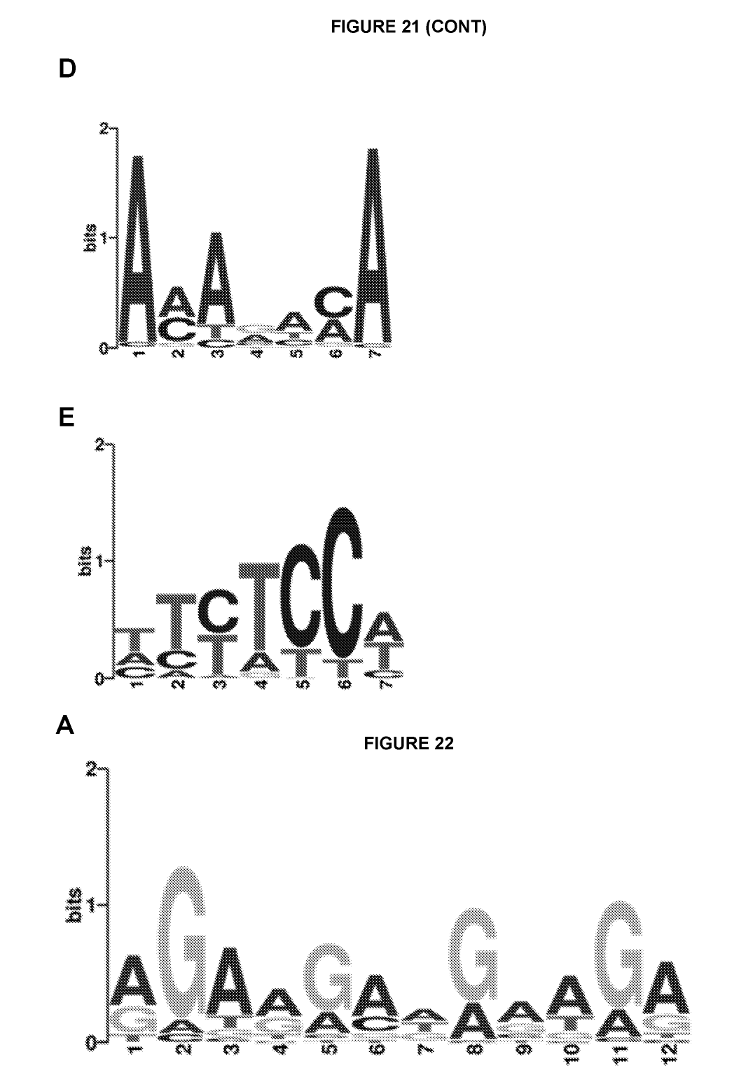

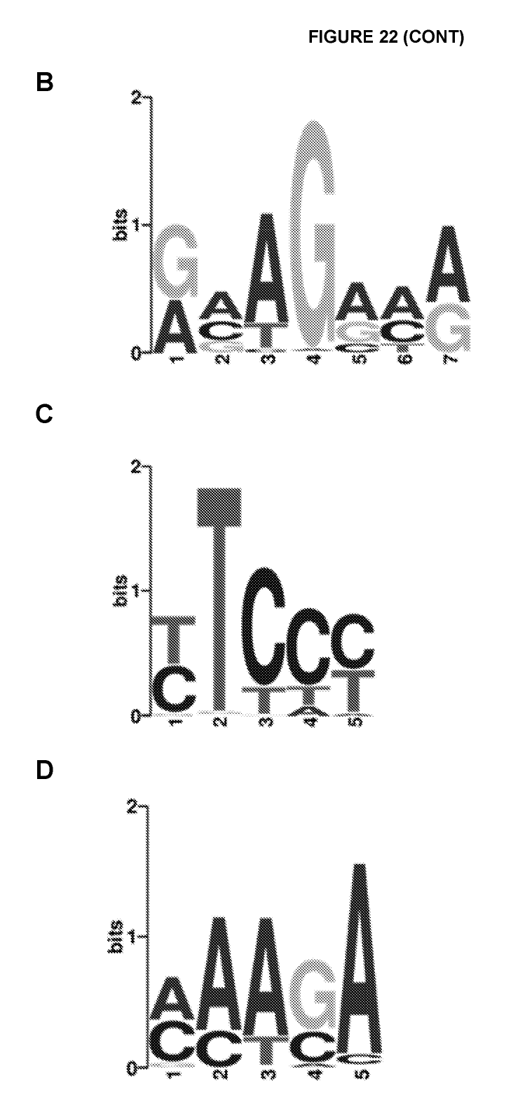

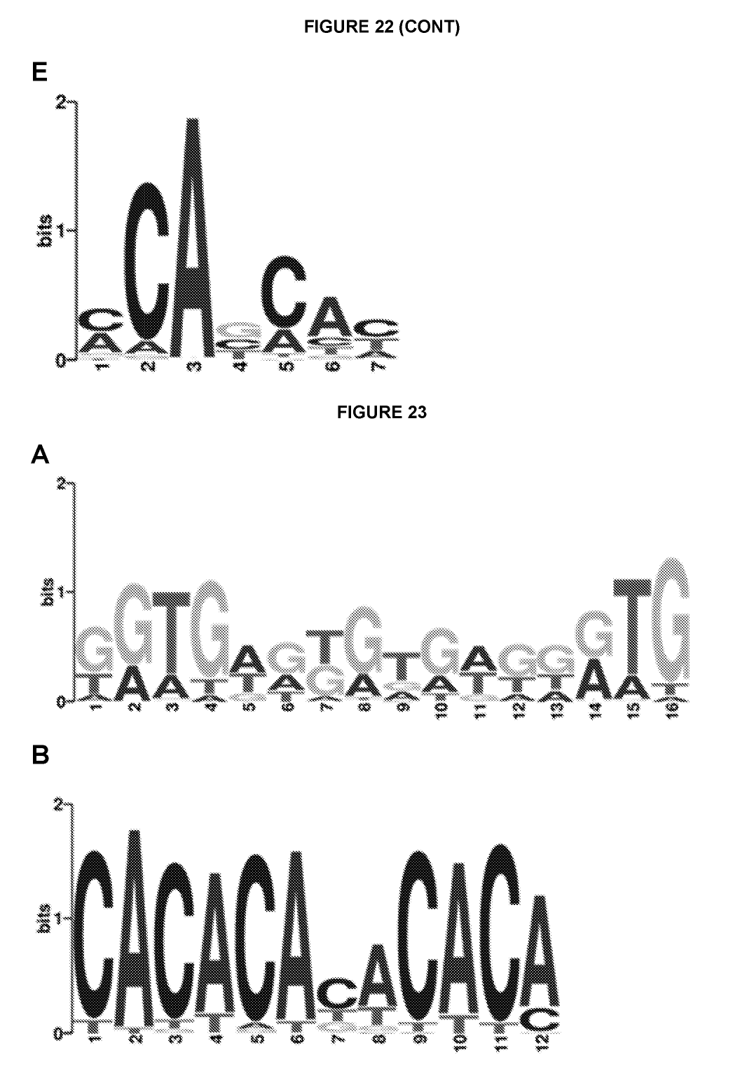

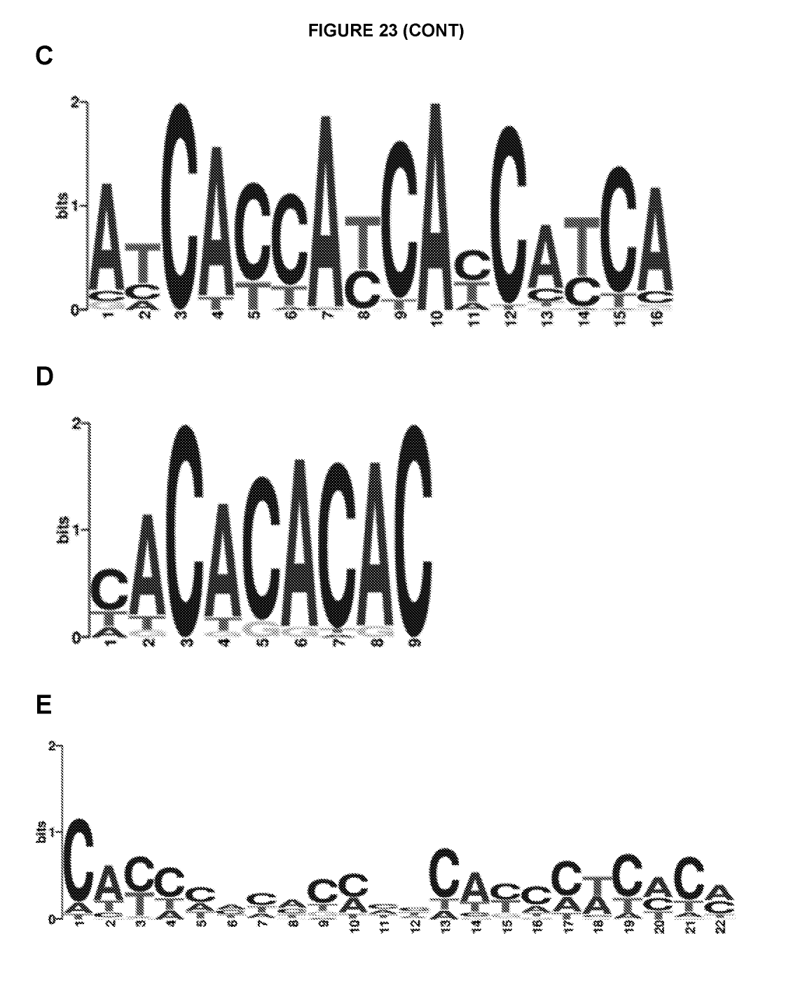

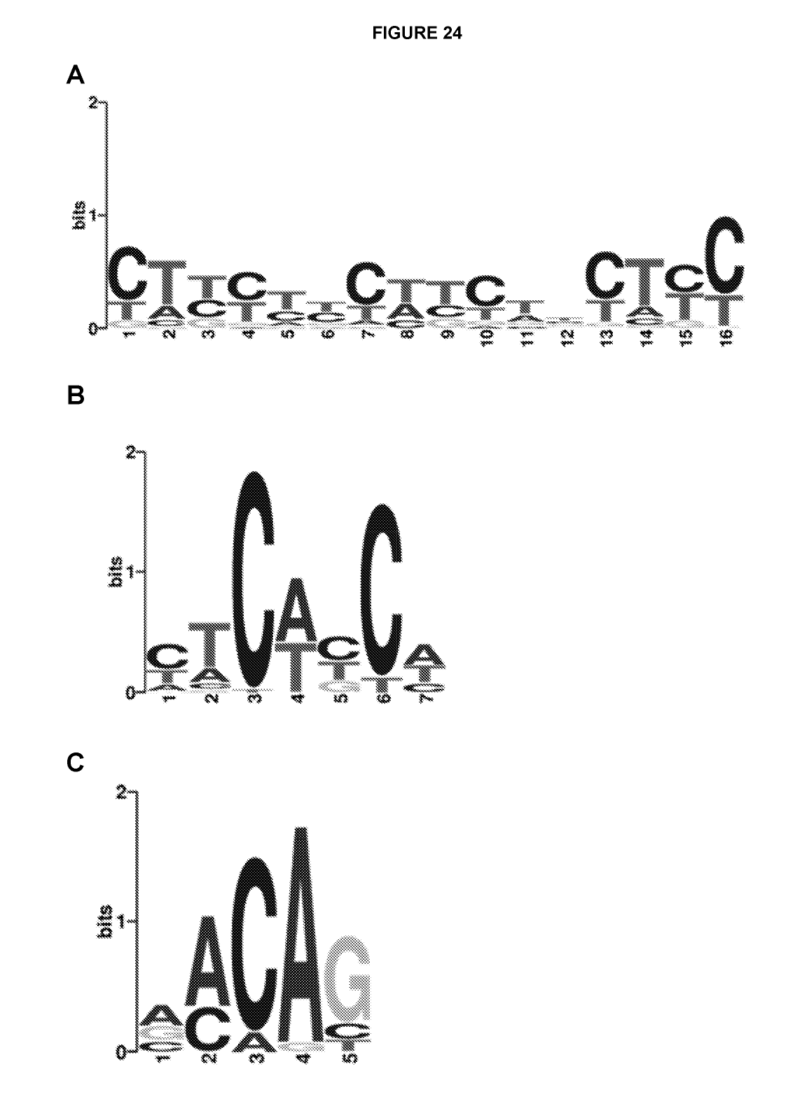

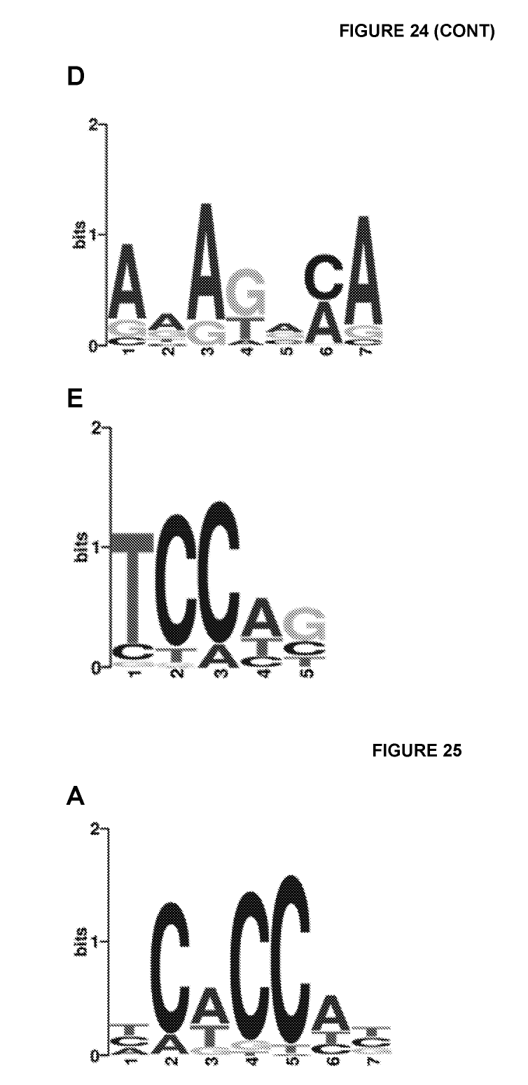

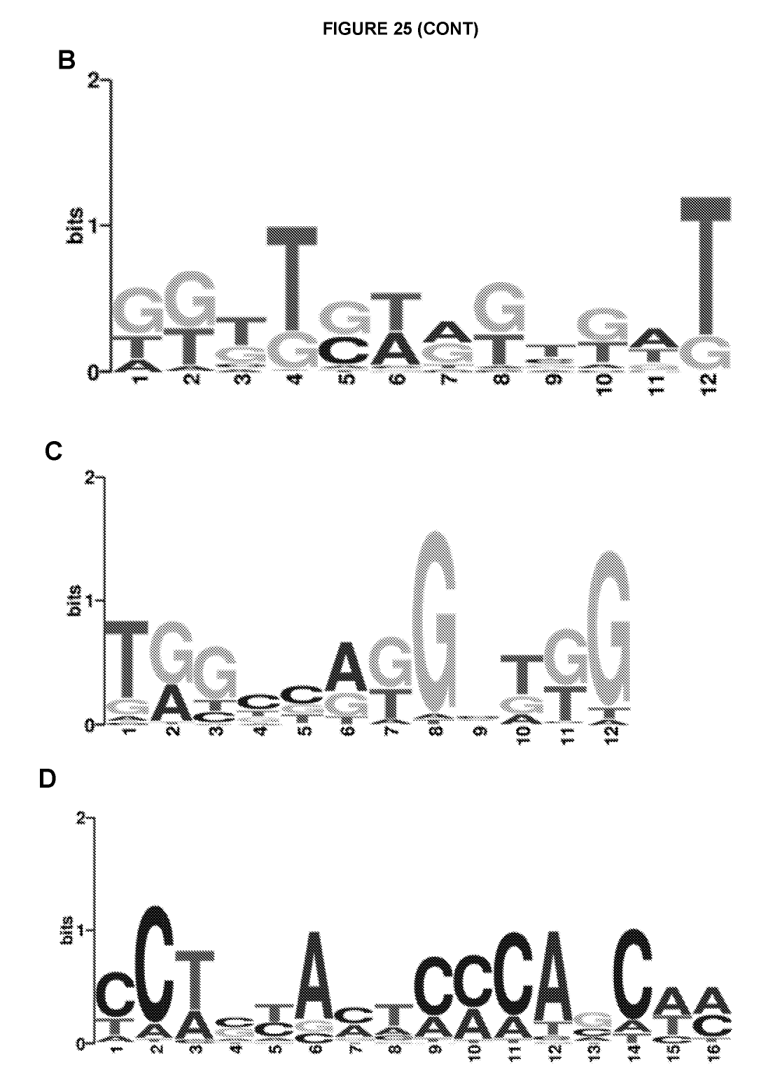

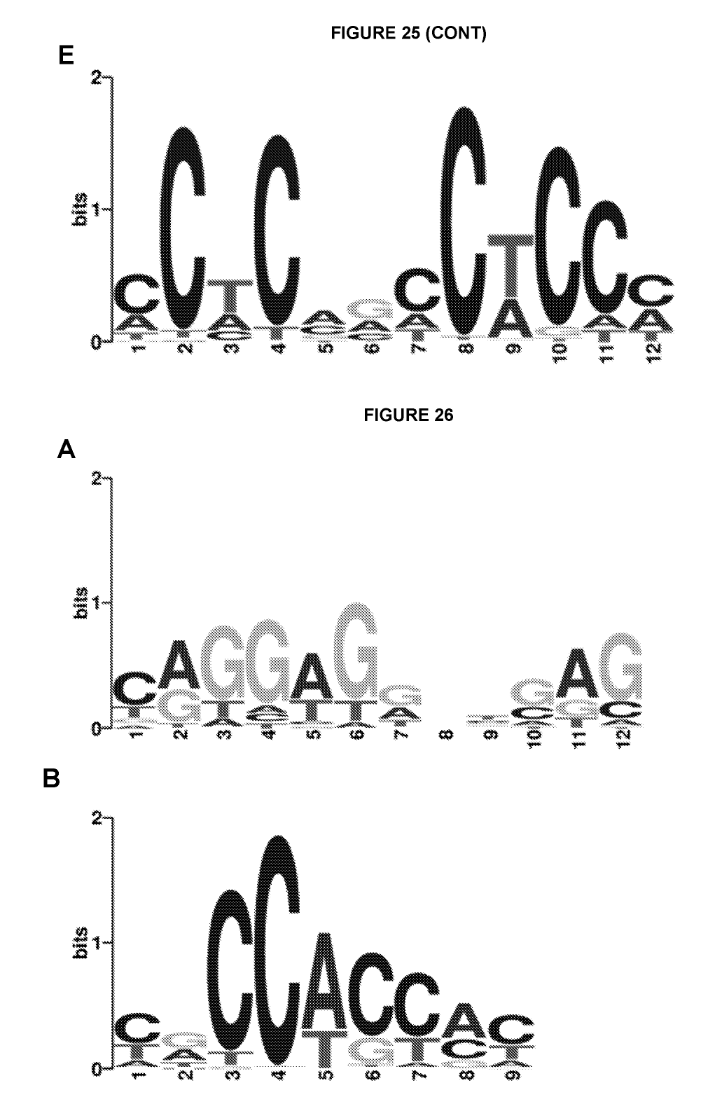

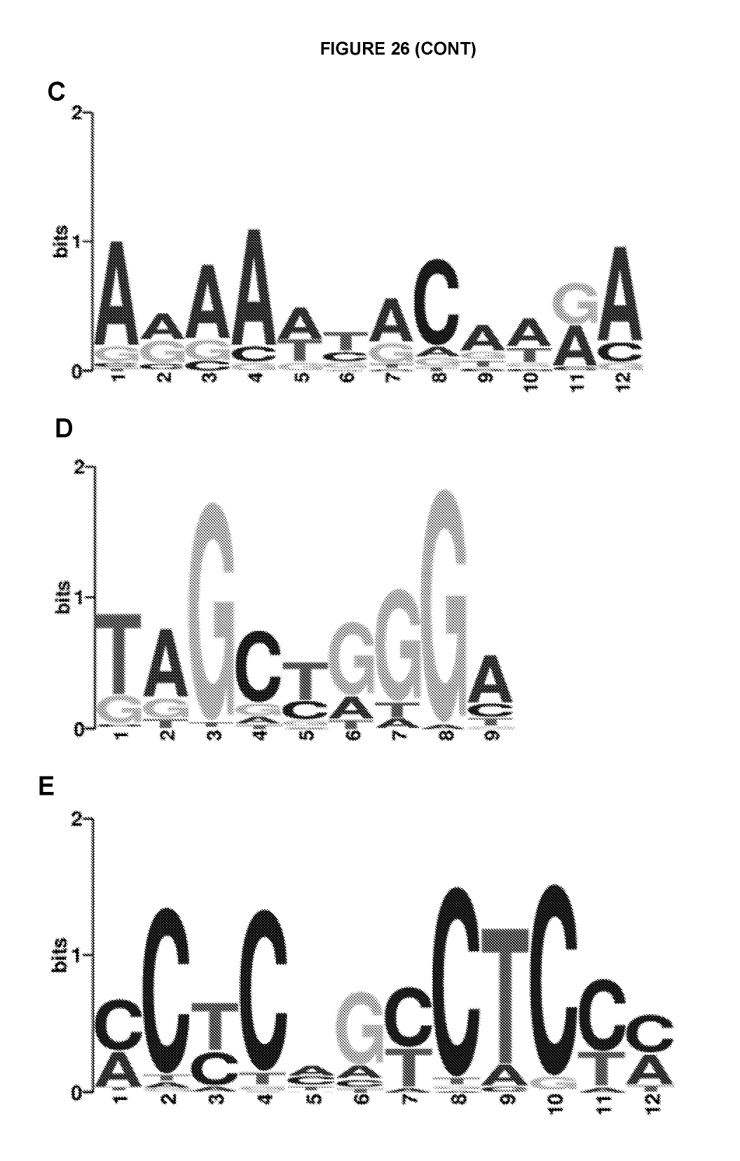

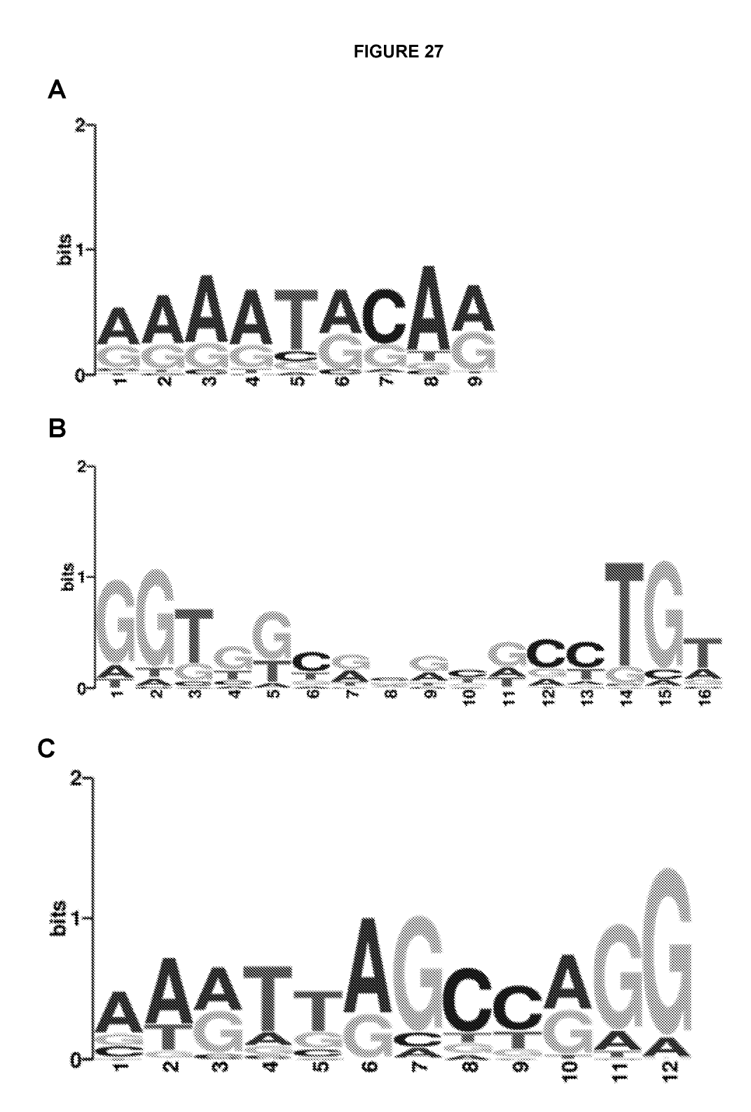

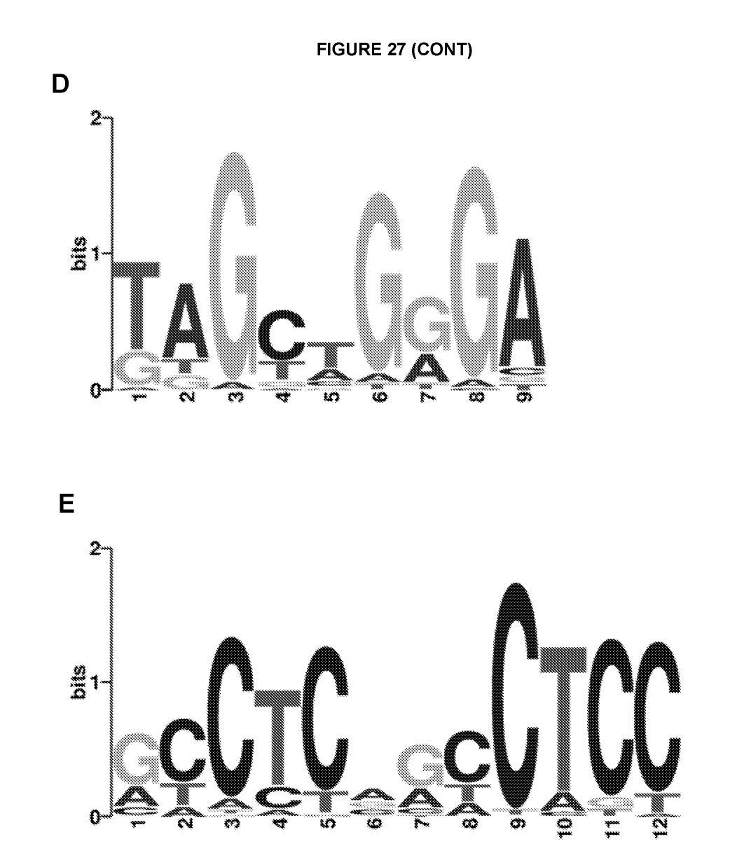

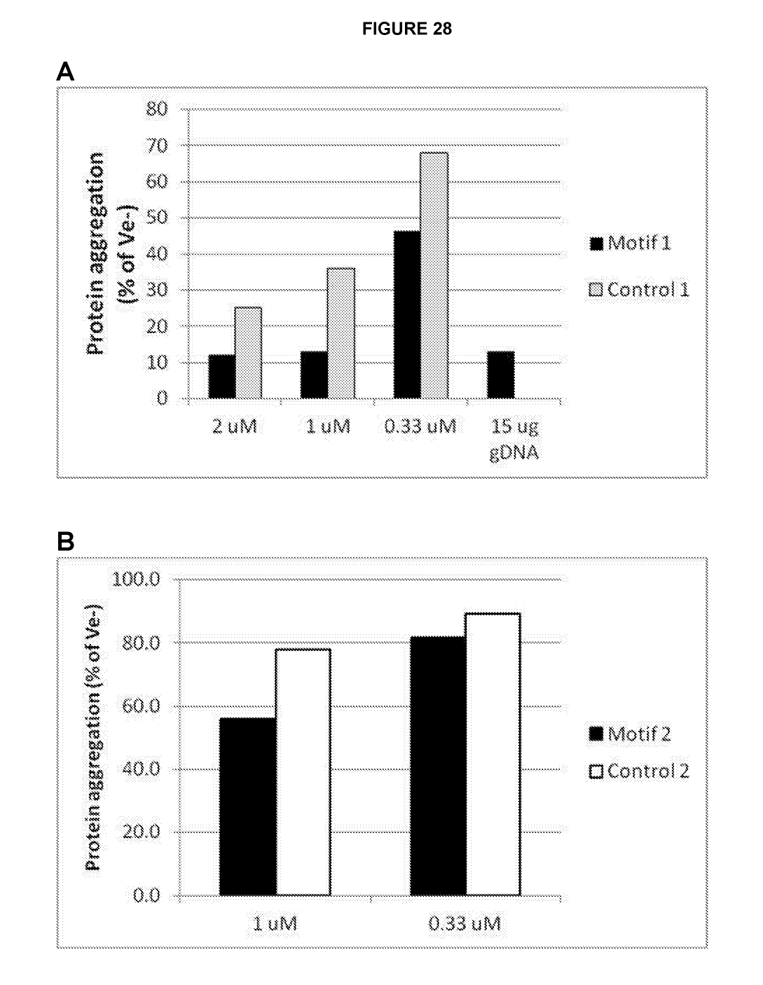

Correct folding is required for the proper function of proteins. To investigate if refolded proteins were functional and not just solubilised by RNA, the activity of ATP-binding proteins, which represent a large proportion of the RNAse aggregated proteins (256/1659) and includes, amongst others, kinases (e.g. GSK3b), ATPases (e.g. actin) and helicases, was assessed. As the ability to bind ATP depends on the presence of conserved structural motifs (often referred to as Walker motifs) as well as phosphate-binding structural features, we first investigated if the ability to bind ATP was restored after refolding in the presence or absence of RNA. Consistent with a requirement for RNA for proper folding, only proteins re-folded with RNA were able to bind ATP while, in contrast, no proteins refolded without RNA bound to ATP (FIG. 15A). Mass spectrometry analysis of the ATP-captured proteins show a clear enrichment of ATP binding proteins, with more than forty percent (58/144) of the identified proteins annotated as ATP-binding, compared to the approximately 15% (256/1659) identified in the initial characterisation. Almost all ATP-captured proteins (130/140) were identified in the initial mass spectrometry analysis (see above), reinforcing the notation that a consistent set of proteins requires RNA for their solubility. RNA-refolded proteins hydrolysed 100-times more ATP than proteins refolded without RNA, whose activity was close to baseline (FIG. 15B). Concomitant removal of RNA by the addition of RNAse A/T1 significantly hampered the ATP-hydrolysing activity of proteins refolded with RNA but had no effect on the proteins refolded with vehicle (FIG. 15B). Similar results were obtained with RNAse-aggregated proteins from Jurkat cells (FIG. 15C). This shows that RNA is required for the functional folding and activity of the ATP-binding and hydrolysing proteins in our mixture.

TABLE 3
Transmembrane proteins aggregated by RNA removal.

| Gene | |
|---|---|
| name | Description |
| ATP13A1 | Isoform B of Probable cation-transporting ATPase 13A1 |
| | OS = Homo sapiens GN = ATP13A1 |
| ATP1A1* | Sodium/potassium-transporting ATPase subunit alpha-1 |
| | OS = Homo sapiens GN = ATP1A1 PE = 1 SV = 1 |
| ATP1A3 | Sodium/potassium-transporting ATPase subunit alpha-3 |
| | OS = Homo sapiens GN = ATP1A3 PE = 3 SV = 1 |
| ATP2B1 | Isoform B of Plasma membrane calcium-transporting |
| | ATPase 1 OS = Homo sapiens GN = ATP2B1 |
| ATP6V1H | ATPase, H+ transporting, lysosomal 50/57 kDa, |
| | V1 subunit H, isoform CRA_c OS = Homo sapiens |
| | GN = ATP6V1H PE = 4 SV = 1 |
| ATP9A* | Probable phospholipid-transporting ATPase IIA |
| | OS = Homo sapiens GN = ATP9A PE = 1 SV = 3 |
| CLIC1 | Chloride intracellular channel protein 1 |
| | OS = Homo sapiens |
| | GN = CLIC1 PE = 1 SV = 4 |
| CLIC4 | Chloride intracellular channel protein 4 |
| | OS = Homo sapiens |
| | GN = CLIC4 PE = 1 SV = 4 |
| GOLT1B | Golgi transport 1 homolog B (S. cerevisiae), |
| | isoform CRA_c OS = Homo sapiens |
| | GN = GOLT1B PE = 4 SV = 1 |
| IFT172 | Isoform 2 of Intraflagellar transport |
| | protein 172 homolog |
| | OS = Homo sapiens GN = IFT172 |
| MAGT1 | Magnesium transporter protein 1 |
| | OS = Homo sapiens |
| | GN = MAGT1 PE = 1 SV = 1 |
| SEC16A | Isoform 2 of Protein transport protein Sec16A |
| | OS = Homo sapiens GN = SEC16A |
| SEC23A | Protein transport protein Sec23A OS = Homo sapiens |
| | GN = SEC23A PE = 4 SV = 1 |
| SEC23B | Protein transport protein Sec23B OS = Homo sapiens |
| | GN = SEC23B PE = 1 SV = 2 |
| SEC24A | Protein transport protein Sec24A OS = Homo sapiens |
| | GN = SEC24A PE = 1 SV = 2 |
| SEC24C | Protein transport protein Sec24C OS = Homo sapiens |
| | GN = SEC24C PE = 4 SV = 1 |
| SEC61A1 | Protein transport protein Sec61 subunit alpha isoform 1 |
| | OS = Homo sapiens GN = SEC61A1 PE = 2 SV = 1 |
| SEC61A2 | Isoform 2 of Protein transport protein Sec61 subunit alpha |
| | isoform 2 OS = Homo sapiens GN = SEC61A2 |
| SLC16A1 | Monocarboxylate transporter 1 OS = Homo sapiens |
| | GN = SLC16A1 PE = 1 SV = 3 |
| SLC25A1 | Tricarboxylate transport protein, mitochondrial |
| | OS = Homo sapiens GN = SLC25A1 PE = 1 SV = 2 |
| SLC2A1 | Solute carrier family 2, facilitated glucose |
| | transporter member 1 OS = Homo sapiens |
| | GN = SLC2A1 PE = 3 SV = 1 |
| SLC39A7 | Zinc transporter SLC39A7 OS = Homo sapiens |
| | GN = SLC39A7 PE = 4 SV = 1 |
| SLC7A5 | Large neutral amino acids transporter small subunit 1 |
| | OS = Homo sapiens GN = SLC7A5 PE = 1 SV = 2 |
| TIGD1 | Tigger transposable element-derived protein 1 |
| | OS = Homo sapiens GN = TIGD1 PE = 2 SV = 1 |
| TNPO1 | Isoform 2 of Transportin-1 OS = Homo sapiens |
| | GN = TNPO1 |
| TNPO3 | Isoform 3 of Transportin-3 OS = Homo sapiens |
| | GN = TNPO3 |
| USO1 | Isoform 2 of General vesicular transport factor p115 |
| | OS = Homo sapiens GN = USO1 |
| VDAC1 | Voltage-dependent anion-selective channel protein 1 |
| | OS = Homo sapiens GN = VDAC1 PE = 1 SV = 2 |
| VDAC2 | Voltage-dependent anion-selective channel protein 2 |
| | OS = Homo sapiens GN = VDAC2 PE = 2 SV = 1 |
| VDAC3 | Voltage-dependent anion-selective channel protein 3 |
| | OS = Homo sapiens GN = VDAC3 PE = 4 SV = 1 |
| CELSR2 | Cadherin EGF LAG seven-pass G-type receptor 2 |
| | OS = Homo sapiens GN = CELSR2 PE = 2 SV = 1 |
| EPHB2 | EPH receptor B2 OS = Homo sapiens |
| | GN = EPHB2 PE = 4 SV = 1 |
| IGF2R | Cation-independent mannose-6-phosphate receptor |
| | OS = Homo sapiens GN = IGF2R PE = 1 SV = 3 |
| KDELR1 | ER lumen protein retaining receptor 1 |
| | OS = Homo sapiens GN = KDELR1 PE = 1 SV = 1 |
| LBR | Lamin-B receptor OS = Homo sapiens |
| | GN = LBR PE = 1 SV = 2 |
| LRP1 | Prolow-density lipoprotein receptor-related protein 1 |
| | OS = Homo sapiens GN = LRP1 PE = 1 SV = 2 |
| MRC2 | C-type mannose receptor 2 OS = Homo sapiens |
| | GN = MRC2 PE = 1 SV = 2 |
| PTPRZ1 | Receptor-type tyrosine-protein phosphatase zeta |
| | OS = Homo sapiens GN = PTPRZ1 PE = 4 SV = 1 |
| SRPRB | Signal recognition particle receptor subunit beta (Fragment) |
| | OS = Homo sapiens GN = SRPRB PE = 4 SV = 1 |
| TFRC | Transferrin receptor protein 1 OS = Homo sapiens |
| | GN = TFRC PE = 1 SV = 2 |
| TGFBR1 | TGF-beta receptor type-1 OS = Homo sapiens |
| | GN = TGFBR1 PE = 2 SV = 1 |
| TOMM22 | Mitochondrial import receptor subunit |
| | TOM22 homolog OS = Homo sapiens |
| | GN = TOMM22 PE = 1 SV = 3 |
| TOMM40 | Mitochondrial import receptor subunit TOM40 |
| | homolog OS = Homo sapiens |
| | GN = TOMM40 PE = 1 SV = 1 |

*indicates proteins that bind ATP after refolding with RNA.

### Example 8—Investigating which Type of RNA was Responsible for Maintaining Protein Solubility

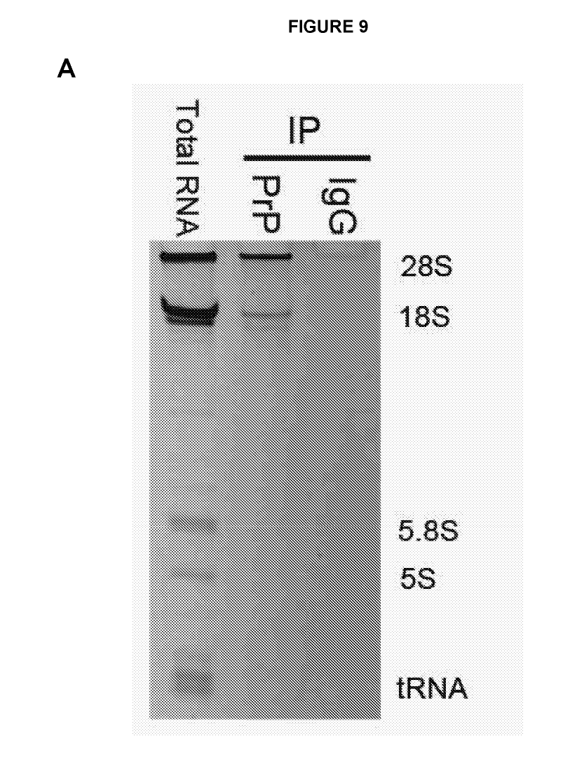

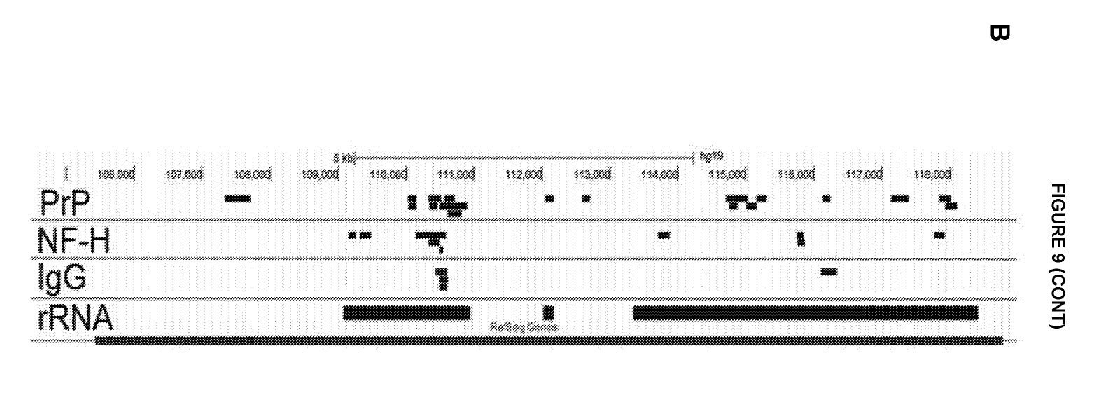

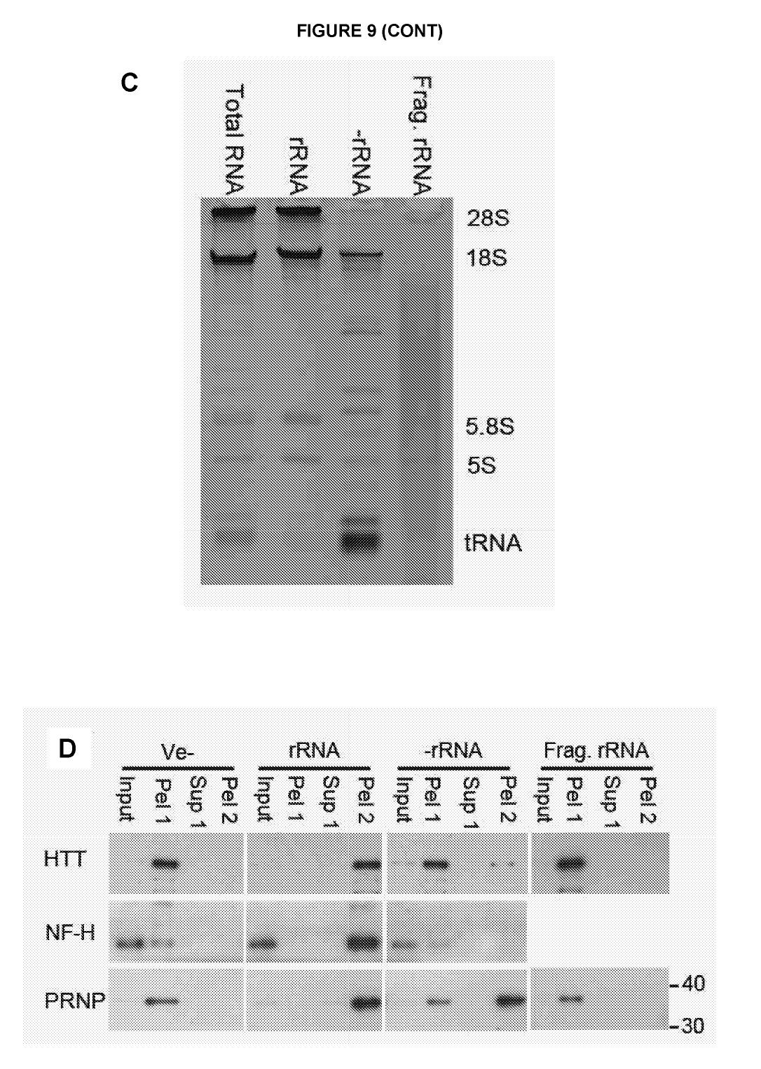

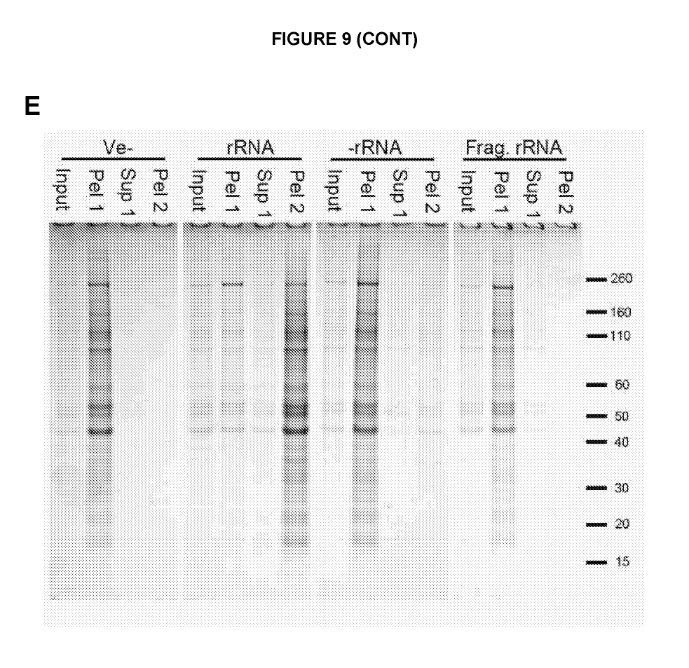

To identify which type of RNA was responsible for maintaining protein solubility, RNA immunoprecipitation of PrP was used from formaldehyde cross-linked cells. PrP was chosen as it lacks conventional RNA-binding domains. Gel-electrophoresis analysis of PrP precipitated RNA showed robust signals from ribosomal RNA (rRNA, 28S and 18S), while no RNAs of any size were precipitated by non-specific IgG antibodies (FIG. 9a). This suggests that PrP is associated with rRNA in cells. To confirm this interaction, the immunoprecipitation was repeated on the soluble fraction of PrP re-folded in the presence of total RNA, as any RNA in this fraction should contain the RNA(s) required for PrP solubilisation. After conversion to cDNA and cloning, 18 of 20 PrP clones in were derived from rRNA, while, in contrast, only 4 clones of 20 (20%) were from rRNA in the IgG sample (FIG. 9b). Thus, soluble PrP associates with rRNA both in vivo and in vitro. RNA associated with NF-H after re-folding was also immunoprecipitated, and similar to PrP, 45% of the sequenced clones (9/20) were from rRNA (FIG. 9b). For all samples, the clones not derived from rRNA were from unique transcripts and thus showed no enrichment (data not shown).

To confirm that rRNA can maintain the soluble state of PrP and NF-H, aggregated proteins were then re-folded in the presence of RNA enriched in, or partially depleted of, rRNA (FIG. 9c). Consistent with a requirement for ribosomal RNA, rRNA efficiently re-solubilised PrP and NF-H, while PrP and NF-H treated with the same amounts of rRNA-depleted RNA precipitated (FIG. 9d). Furthermore, limited chemical fragmentation of rRNA (FIG. 9c) before re-folding, efficiently prevented solubilisation of PrP and NF-H (FIG. 9d). Together, these findings show that intact rRNA is required for efficient re-solubilisation of these proteins. Interestingly, after re-folding in the presence of RNA enriched in rRNA no visible pellet was detectable after centrifugation (data not shown). This suggested that several proteins were maintained in a soluble state by associating with rRNA. To confirm this, the experiment was repeated and the global protein profile was analysed by gel electrophoresis. Similar to PrP and NF-H (FIG. 9e), the majority of the RNAse-precipitated proteins, including HTT, efficiently re-folded in the presence of rRNA, but crucially, not when re-folded with fragmented rRNA or RNA samples depleted of rRNA (FIG. 9d, e). The fact that limited fragmentation of RNA prevents solubilisation of the proteins suggests that they do not primarily associate with a particular RNA sequence, since the same sequences, at similar levels as in intact rRNA, are present in the fragmented rRNA (FIG. 9c). Rather, it suggests that the structure of the rRNA is critical for the solubilisation of the proteins, a notion also supported by the efficient precipitation of proteins from cell lysates by RNAse V1 (FIG. 6d), a ribonuclease specific for double-stranded RNA. This structural requirement could also explain the efficient solubilisation of aggregated proteins by genomic DNA (FIG. 8h), which, in its A-form, is structurally similar to double-stranded RNA.

### Example 9—Prion-Like Spreading

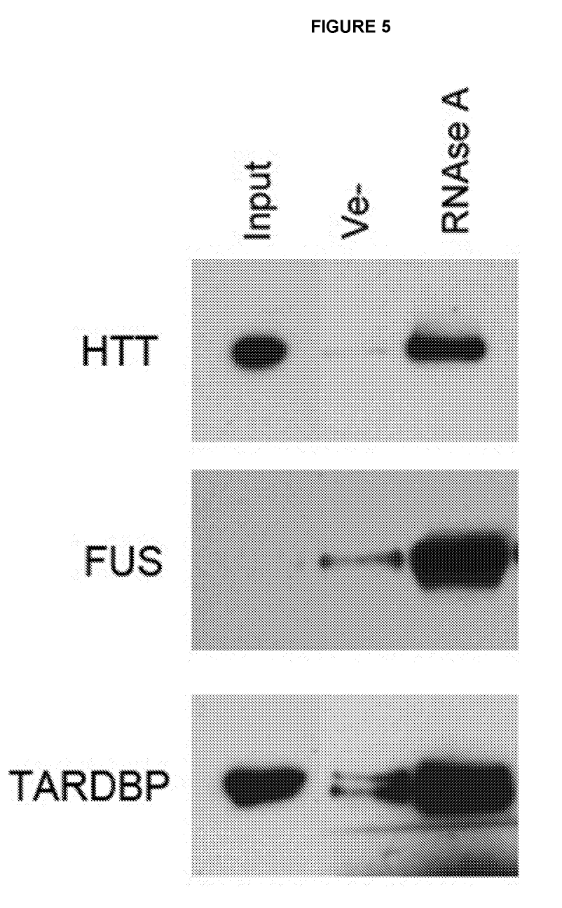

As explained above, recent evidence suggests that protein aggregates in neurodegenerative diseases have the capacity to self-propagate (i.e. spread) by a prion-like mechanism. In order to test this, protein aggregation was initiated in a sample by RNA removal, and then a fraction of this sample was mixed with a non-treated lysate. Aggregated proteins were isolated by centrifugation, solubilised in SDS, separated on SDS-PAGE gels and probed by antibody. The Results are shown in FIG. 5. An increase in aggregation was seen in the RNAse treated samples which is thought to be due to propagation of the aggregation, e.g. recruitment of native proteins in the non-treated sample.

### Example 10—G-Quadruplex Structures

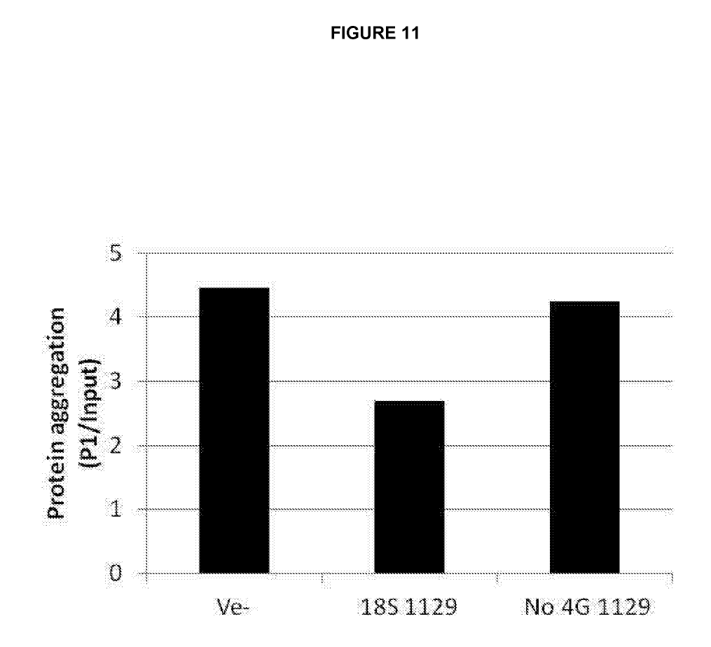

Both DNA and RNA can form a particular structure called G-quadruples, G4 (Nucleic Acids Research, 2008, Vol. 36, pp 5482-5515). The inventors have synthesised a predicted G4-forming ssDNA oligo (Table 2) derived from human ribosomal RNA and tested it in the refolding assay described herein. The data indicate that this oligo can solubilise the proteins; while an oligo with the same nucleotides but in a non-G4-permissive order, is much less efficient (FIG. 11, Table 2).

TABLE 2
Nucleotide sequences of predicted and
non-permissive G-quadruple forming oligos.
18S 1129-69 indicate that the sequence is from
the human 18S ribosomal RNA and spans the
position from 1129 to 1169. No 4G means that it
is not predicted to form G-quadruples.

| | Size | |
|---|---|---|
| Name | (nt) | Sequence |
| 18S | 40 | GGG CAG CTT CCG GGA AAC CAA AGT CTT |
| 1129-69 | | TGG GTT CCG GGG G |
| | | [SEQ ID NO: 1] |
| No 4G | 40 | TTA AGG GCA TCT GAT GCG GCG CGT GAC |
| 1129-69 | | GCG GCG CTA AGT G |
| | | [SEQ ID NO: 2] |

### Example 11—Sodium Orthovanadate Inhibits RNAse Mediated Protein Aggregation

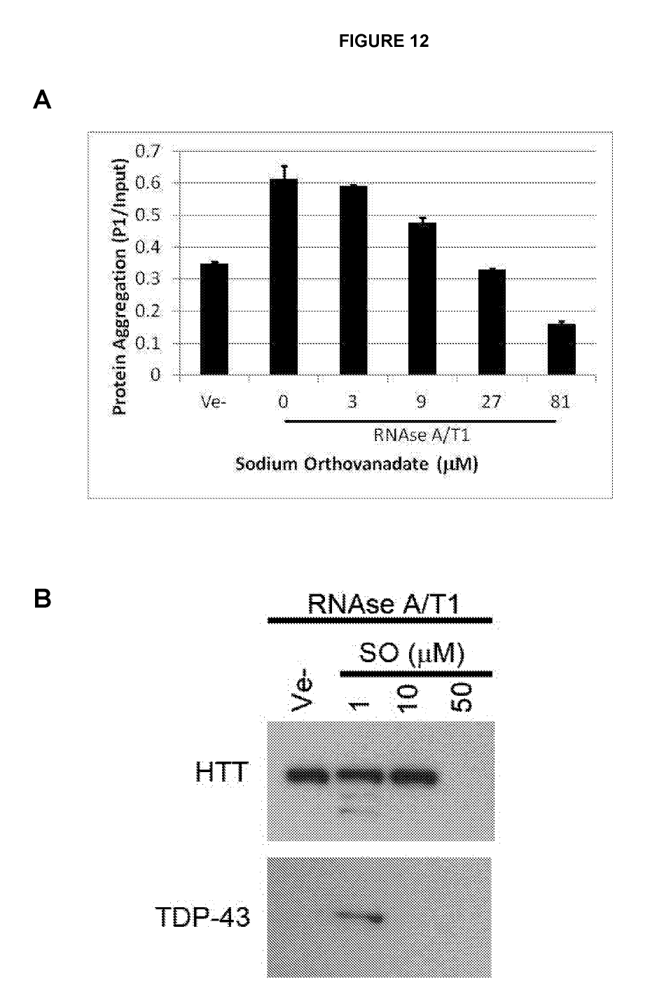

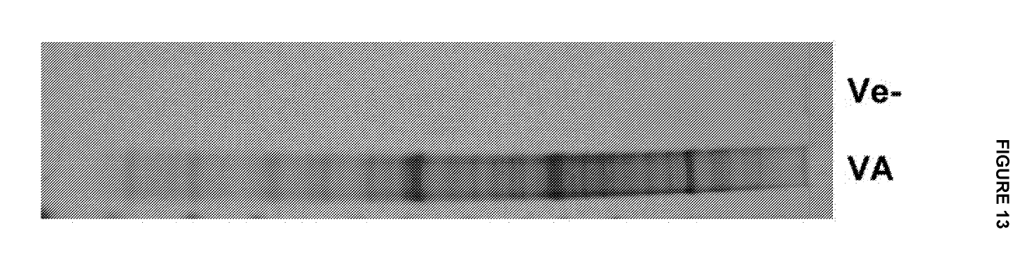

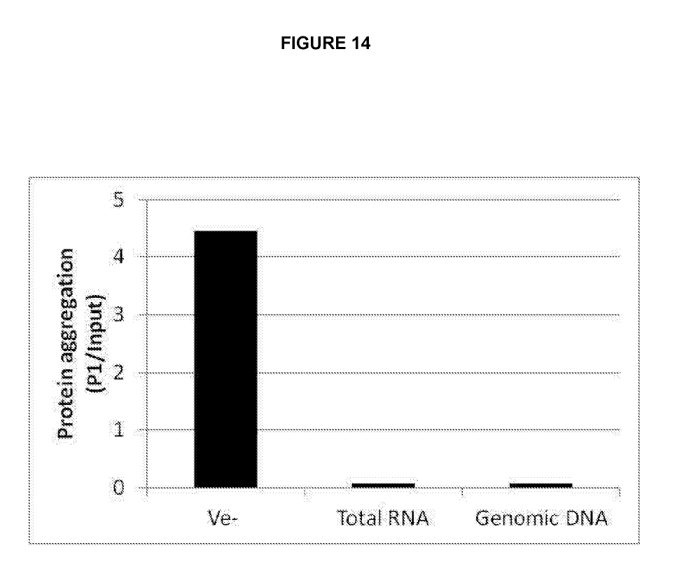

Sodium orthovanadate (SO) is a small molecule that is primarily used to inhibit protein phosphatases. When added to cell or tissue lysate together with RNAses it can completely inhibit aggregation of the proteins, for example of TDP-43 and huntingtin (FIGS. 12a and 12b).

Addition of SO also prevents any background protein aggregation, which the inventors' previous data indicate is caused by endogenous ribonucleases.

### Example 13—Ribonucleoside Vanadyl Induced Protein Aggregation

Ribonucleoside vanadyl (RV) is a molecule used as a transition state analogue inhibitor of RNAses during RNA purification and manipulation. However, when added to lysate in the absence of any exogenous RNAse, it causes substantial protein aggregation (FIG. 13).

RV therefore provides an alternative method for inducing protein aggregation.

### Material and Methods

### Enzymes.

RNAse T1, RNAse V1, RNAse A/T1 cocktail, and DNAse I were from Life Technology. RNAse A was from Sigma.

### Cell Cultures.

Neurons were differentiated from human neural stem cells by withdrawal of basic FGF for 6 days. The majority (>95%) of the cells differentiate into Map2- and β III-tubulin-positive cells within 6 days. Jurkat T cells were maintained in RPMI (Life Technologies) supplemented with 10% FCS (Life Technologies) and 1× Pen/Strep (Life Technologies).

Cell Free Lysates from Neurons and Mouse Cortex.

Differentiated neural stem cells were detached by trypsin (0.5% Life Technologies) and collected in RPMI medium with 10% FCS (Life Technology). Cells were pelleted by centrifugation and washed twice in ice-cold PBS before being lysed in four cell-pellet volumes of either Lysis Buffer 1 [20 mM Tris-HCl pH 7.5, 150 mM NaCl, 3 mM EDTA, 1% Triton X-100, 0.5% Na-Deoxychoalte, 1× protease inhibitors cocktail (Roche), 1 mM DTT] or Lysis Buffer 2 [20 mM Tris-HCl pH 7.5, 150 mM NaCl, 1.5 mM MgCl2, 1% Triton X-100, 0.5% Na-Doxycholate, 1× protease inhibitors cocktail (Roche), 1 mM DTT]. Most experiments were performed in Lysis Buffer 1, except when RNAse V1 treatment was performed (FIG. 1e), in which case Lysis Buffer 2 was used. Lysed cells were sonicated (Bioruptor, Diagenode) at maximum setting for 5 seconds on ice and centrifuged at 21.000×g for 30 min at +4° C. The supernatant was transferred to new tubes and the protein concentration determined with the BCA kit (Thermo Fisher) according to the manufacturer's instructions. Lysates were diluted in Lysis Buffer-1 or -2 to 2-4 μg/μl and treated as described below.

Cortices from day 16-21 C56BL mice were dissected at room temperature, rolled on filter paper to remove most of the meninges and immediately frozen on dry ice and stored at −80° C. until use. The tissue was thawed on ice and disrupted in cold PBS using a 1 ml pipette tip. Disrupted tissue was washed 3 times in PBS before being lysed in Lysis Buffer 1 and prepared as described for human neurons.

### Ribonuclease Treatment and Isolation of Precipitated Proteins.

Typically, 200-400 μg cell lysafe at 2-4 μg/μl was mixed with indicated amounts of ribonucleases, DNAse I, or Vehicle (50% Glycerol in 20 mM Tris-HCl pH 7.5) and incubated at 37° C. for one hour. Samples were then centrifuged at 21.000×g for 15 min at +4° C. and the supernatants removed and saved for analysis. The pellets were washed twice in 500 μl RIPA buffer at room temperature (50 mM Tris-HCl pH 8.0, 150 mM NaCl, 0.5% Na-deoxycholate, 0.1% SDS, 1% Triton X-100) and dissolved in 20 mM Tris-HCl pH 7.5, 2% SDS, 8M Urea by sonication (Bioruptor, Diagenode) 5 min at room temperature. Samples for SDS-PAGE analysis were mixed with 4×LDS Loading Buffer (Life Technologies) supplemented with DTT to 100 mM final concentration and heated for 10 min at 70° C. before being loaded on SDS-PAGE gels (Life Technologies).

### Immobilisation of RNAse A.

100 μg RNAse A at 1 μg/μl was coupled to sylactivated magnetic beads (Life Technologies) for 20 hours at 37° C. according to the manufacturer's instructions. After quenching and washing the coupled RNAse A was re-suspended in 0.1% BSA in PBS and kept at +4° C. until use. Approximately 50% activity remained after coupling, as determined on yeast tRNA using the RiboGreen kit (Life Technologies).

### Inhibition of RNAse a and Addition of Pre-Hydrolysed RNA.

RNAse A inhibition: 200 μg lysate was mixed with 0.1 μl RNAse A (˜3 mg/ml) and increasing concentrations of RNasin (Promega), as indicated. Hydrolysis of RNA: 40 μg of total RNA in TE-buffer (10 mM Tris-HCl pH 7.5, 1 mM EDTA) was incubated with 10 μl immobilised RNAse A for 1 hour at 37° C. RNAse A was removed by magnetic separation and the hydrolysed RNA was mixed with 120 U RNasein (Promega) and kept on ice until used. Alternatively, 40 μg total RNA in 0.1 M NaOH was incubated at 85° C. for 1 hour and then adjusted to pH 7.5 with 1 M Tris-HCl pH 7.0. RNAse A digested and NaOH hydrolysed RNA was then added to 200 μg of neuronal lysate, prepared as outlined above, and incubated at 37° C. for one hour. Precipitated and soluble proteins were collected as before and analysed by SDS-PAGE.

### Cloning and Precipitation of Aβ.

Human Aβ 1-40 was PCR amplified from full length APP (Origen, #RC209575) and cloned into the Xho I and Bam HI sites of pEGFP-C3 (Clontech), creating Aβ fused in frame to the C-terminus of GFP. HEK293 cells, plated at a density of 0.2×106 cells/well in a 24 well plate, were transfected with Aβ-GFP or empty vector using Fugene HD (Promega). For each well we used 0.6 μg DNA and 2 μl Fugene HD in a total volume of 30 μl OptiMEM (Life Technologies). Cells were harvested 48 hours after transfection and washed in PBS and then either stored at −80° C. or used directly. Thawed or fresh cells were lysed in 80 μl Lysis Buffer 1 as described above and treated with RNAse A/T1. Aggregated proteins were collected by centrifugation and samples process and analysed by SDS-PAGE as described above.

### RNA-Mediated Re-Folding.

Proteins were isolated from neuronal lysate by RNAse A/T1 treatment and centrifugation. Pelleted proteins were dissolved in 50 μl of denaturation buffer (20 mM Tris-HCl pH 7.5, 6 M Guanidine hydrochloride, 1% Triton X-100, 20 mM DTT) and sonicated for 5 min at room temperature. The protein concentration was determined with the BCA kit (ThermoFisher) and diluted to 0.4 μg/μl in denaturation buffer. 20-50 μg of solublised proteins was mixed with 0.5×, in μg, of RNA in TE buffer and transferred to dialysis tubes (see below) equipped with a 6-8.000 kDa cut-off membrane (Spectrum Lab). Dialysis was performed against 600 ml PBS buffer at 4° C. overnight after which the PBS was replaced with fresh PBS (400 ml) and the container placed in a water bath and kept at 37° C. for 1 h. The dialysed samples were transferred to 1.5 ml tubes and the volume adjusted to 100-200 μl with PBS. 7.5-10% of this was taken as Input. Precipitated proteins (Pel 1) were pelleted by centrifugation at 21.000×g for 10 min at +4° C., washed twice in RIPA buffer and processed for SDS-PAGE as before. 7.5-10% of the supernatants was saved (Sup 1) and the remaining volume was either divided into two new tubes supplemented with 0.5 μl vehicle or 0.5 μl RNAse A/T1 or the whole sample placed in one tube and treated with 0.5 μl RNAse A/T1. All samples were incubated at 37° C. for one hour and centrifuged as before.

Pelleted proteins (P2) were washed as before and dissolved in SDS/Urea and sonicated. Equal volumes of each fraction were separated on SDS-PAGE gels and then either stained with coomassie or transferred to membranes for western blot analysis.

### ATP-Binding and Hydrolysis.

100 μg RNAse aggregated proteins from human neurons or Jurkat cells were refolded with 50 μg of total RNA or Ve (TE buffer) as described above. After dialysis the samples were adjusted to 250 μl with PBS and centrifuged at 2,000×g for 15 min. Capturing of ATP binding proteins was performed on 75 μl of this mixture using 30 μl of Aminophenyl-ATP- or naked agarose beads (Jena Bioscience) according to the manufacturer's protocol. Elution was performed by two sequential 10 min incubations in 20 μl 1×LDS loading buffer (Life Technologies) supplemented with DTT (100 mM final concentration). One fourth of the eluted samples was separated on 4-12% NuPage gels (Life Technologies) and the gels stained with coomassie blue (ProtoBlue, National Diagnostics). The remaining eluate from two independent replicates was electrophoresed approximately 1 cm into a 4-12% NuPage and the top piece of the gel excised and prepared for mass spectrometry analysis as described below. To measure ATP hydrolysis, we used the ADP-Glo™ Kinase Assay (Promega) according to the manufacturer's instructions. Briefly, 5 μl of refolded proteins were mixed in a white 96-well plate (Santa Cruz Biotechnology) with ATP (100 uM final concentration) and 0.1 μl RNAse A/T1 mixture or vehicle (50% Glycerol in 20 mM Tris-HCl pH 7.5), all diluted in 1×PBS, 5 mM MgCl2, 2 mM DTT, in a total volume of 15 μl and incubated at room temperature for 1.5 hour. Non-hydrolysed ATP was removed by the addition of 15 μl of ADP-Glo reagent followed by incubation for 1 hour at room temperature. ADP was converted back to ATP by the addition of 30 μl Kinase Detection Reagent and the emitted light quantified after 1.5 hours incubation at room temperature using a Victor2 Multilabel plate-reader (Wallac). All samples were run in duplicate and data presented as the mean of three independent replicates. Statistical analysis was performed with student's t-test (two sample test for mean) available in Excel (Microsoft).

### Dialysis Tubes.

Dialysis tubes were prepared by drilling a 3 mm hole in the lid of a 1.5 ml microcentriruge tube (Crystal Clear, StarLab). The tube was then cut 1 cm from the top and a new, intact lid inserted at the bottom. After sample addition the tube was sealed with a dialysis membrane and capped with the drilled lid. This creates a dialysis tube where one end is in contact with the surrounding solution, separated by the membrane. Tubes were placed in the dialysis solution with the holed side facing down.

### SDS-PAGE and Western Blot Analysis.

Heated samples were separated on 4-12% Bis-Tris gels (Life Technology) in MOPS or MES buffer and either transferred to 0.2 μm nitrocellulose or 0.45 μm PVDF membranes (Both GE Healthcare) for 2 hour at 45V on ice or, alternatively, used directly for coomassie (ProtoBlue, National Diagnostics) staining according to the manufacturer's protocol. After transfer, membranes for Western blot were blocked for one hour at room temperature in 5% milk in TBS-T (50 mM Tris-HCl pH 7.5, 150 mM NaCl, 0.05% Tween-20) and incubated with primary antibodies in the same solution or TBS-T/5% BSA overnight at +4′C. Membranes were then washed 4×5 min in TBS-T and incubated for 1 hour at RT with HRP-conjugated secondary antibodies diluted in 5% milk/TBS-T. Membranes were then washed as before and incubated for 5 min in ECL Prime (GE Healthcare) before being exposed to films (ThermoFisher). Primary antibodies used: TDP-43 (New England Biolabs, NEB, #G400), HTT (NEB, #D7F7), FUS (SantaCruz, #sc-47711), SNCA (NEB, #D37A6), MAPT (NEB, #Tau46), PrP (Proteintech, #12555-1-AP), NF-H (Covance, #SMI-32R), A□ 6E10 (Covance, #SIG-39320), ACTB (Sigma, #A2228), RPL7 (Abcam, #ab72550), PABP (Abcam, #ab21060). All primary antibodies were used at 1:1000 dilution, except PrP (1:2000), ACTB (1:4000), NF-H (1:4000), FUS (1:100), and RPL7 (1:2000). As secondary antibodies we used Donkey anti-Rabbit HRP (#NA934V) or Sheep anti-Mouse HRP (#NXA931), both from GE Healthcare, diluted 1:50,000 in 5% milk-TBS-T.

### RNA Isolation and Analysis.

RNA was isolated from cell lysates and purified ribosomes with Trizol LS (Life Technologies) according to the manufacturer's instructions. Ribosomes were isolated from Jurkat T-cells. RNA depleted for rRNA was isolated from the upper two thirds of the supernatant (after pelleting of ribosomes and five times dilution in water) with acid phenol (Life Technologies) followed by EtOH precipitation. RNA from intact cells was isolated with Isol-RNA Lysis Reagent (5 PRIME), according to the manufacturer's instructions. All RNA samples were dissolved in TE (10 mM Tris-HCl pH 8.0, 1 mM EDTA). RNA was analysed by 1.5% agarose or 6% PAGE/8M Urea gel electrophoresis and visualised with Ethidium Bromide. To fragment rRNA, 50-100 μg RNA in 10 mM ZnCl2, 10 mM Tris-HCl pH 7.0 was incubated at 70° C. for 7 min, mixed with 1:50 volume of 0.5 M EDTA and then EtOH precipitated. All re-folding experiments were performed with freshly prepared RNA from human neurons or Jurkat T-cells. Before mixing with the solubilised proteins, RNA samples were heated to 65° C. for 5 min and then cooled on ice for at least 3 hours before being used.

### Mass Spectrometry Analysis.

30 μg of RNAse-precipitated proteins in 1×LDS loading buffer (Life Technologies) supplemented with 100 mM DTT were separated on 4-12% Bis-Tris gels in MOPS running buffer. After coomassie staining, each gel lane was divided into 10 equal gel-slices and cut into 1 mm cubes. Gel bands were destained and reduced with 5 mM TCEP (Pierce) and alkylated with 50 mM chloracetamide (Sigma) and then digested with trypsin (Promega) for 16 hours. Samples were desalted using homemade C18 columns and then analysed using a QExactive mass spectrometer (Thermo, Hemel Hempstead) at the Central Proteomics Facility (University of Oxford, UK). Data were analysed using Mascot (MatrixScience, London) with searches performed against the UniProt Human database. Proteins with a Mascot score greater than or equal to 60 and with two unique peptide sequences were considered to be confidently identified.

Computational analysis of RNAse-precipitated proteins. Proteins common to both MS samples, each with a Mascot score 260, were compiled into a list and used for further analysis. Low complexity regions of 30 or more consecutive amino acids were identified using SEG (REF) using the following parameters: [30][3.2][3.55]. Unstructured regions were identified using DisEMBL (REF) with the following parameters: AA window of 30, join 2, threshold 1.75. The results were compared to those obtained by permutation analyses. A total of 1,000 permutations per analysis was performed. The permutations consisted of random sets of proteins (n=1,603) withdrawn from the complete set of human proteins (http://www.uniprot.org/downloads, accessed on July 2013) analysed all using SEG and DisEMBL. The cumulative distributions of the proportion of low complexity and unstructured regions were compared to the results obtained from RNAse-precipitated proteins.

### RNA Immunoprecipitation and Sequencing.

Molecular crosslinking of 14×106 Jurkat T-cells was achieved with 0.1% formaldehyde in PBS for 10 min at room temperature. The reaction was stopped by the addition of 1:10 volume of 1.5 M Glycine followed by a 10 min incubation on ice. Crosslinked cells were washed twice in cold PBS and lysed in 50 mM Tris-HCl pH 7.5, 250 mM Sucrose, 250 mM KCl, 5 mM MgCl2, 0.7% NP-40 for 15 min on ice. Nuclei were pellet by centrifugation (800×g at +4° C. for 10 min) and discarded. The supernatant was then further centrifuged at 21.000×g for 20 min at +4° C. and then transferred to new tubes. The supernatant was adjusted to 0.5 M KCl and used for immunoprecipitation. Lysate was incubated rotating overnight at +4° C. with 0.3 μg PrP (Proteintech, #12555-1-AP) or rabbit IgG (Sigma) antibodies, and then mixed with 5 μl pre-washed protein A dynabeads (Life Technologies) and incubated for 30 min at room temperature. Beads were washed five times with PBS and twice with PBS supplemented with NaCl to a final concentration of 0.5 M. Samples were eluted at 65° C. for 5 min in 50 μl of 20 mM Tris-HCl, 300 mM NaCl, 1% SDS and the supernatant diluted to 300 □l with 20 mM Tris-HCl pH 7.0, 300 mM NaCl supplemented with 1 ul Proteinase K (Merck). Crosslinking was reversed by incubating the samples for 20 min at 65° C. and RNA was isolated by extraction in acid phenol pH 4.5 (Life Technologies) and ethanol precipitated. Precipitated RNA was dissolved in TE and analysed on 6% PAGE/8 M Urea gels and visualised with ethidium bromide. RNA-IP for cloning was performed on proteins re-folded in the presence of total RNA as described above using anti PrP (Proteintech, #12555-1-AP), NF-H (Covance, # SMI-32), or rabbit IgG (Sigma). Samples were incubated while being rotated for 2 hours at +4° C. and then mixed with either 5 μl washed Protein A beads (PrP) or 5 μl Goat-anti mouse IgG magnetic beads (Life Technologies) (NF-H and IgG) and left rotating for 30 min at room temperature. Beads were washed five times in PBS and once in PBS supplemented with NaCl to 0.5M. Samples were eluted in 1% SDS and RNA extracted with acidic phenol and ethanol precipitated, as described above. Precipitated RNA was converted to double stranded cDNA and PCR amplified using the Illumina TrueSeq kit according to manufacturer's instruction, except that no initial fragmentation was performed. Amplified cDNA was blunt-end ligated into Sma I-cleaved pUC 19 vector (NEB), transformed into E. coli (NEB, DHA5a) and plasmids from single colonies prepared for sequencing. Sequenced clones were mapped using BLAT software. Only the longest matches with a percentage of identity of more than 96 were considered for each clone.

### Sodium Orthovanadate: Impact on RNAse-Induced Protein Aggregation.

Cell or tissue lysate was prepared as described above. Lysate (200 μg at 2 μg/ul) is then treated with a mixture of RNAse A and T1 (A/T1), and incubated with increasing concentrations of Sodium Orthovanadate for one hour at 37° C. Aggregated proteins were isolated by centrifugation (21.000×g) at 4° C. for 20 min, washed twice in 500 μl RIPA buffer and then dissolved in 12 μl 6M Guanidine Hydrochloride, 20 mM Tris-HCl pH 7.5 by sonication for 5 min at room temperature. The amount of protein in 5 μl of this solution was then determined by using the BCA Kit (Thermo Scientific) according to the manufacturer's protocol. Data were expressed in arbitrary units (AU) as a fraction of the amount aggregated proteins (P1)/the amount of protein in the Input, and represent mean±s.d, n=2. Both activated (boiled and pH adjusted) and non-activated SO gave similar results. The experiment was repeated with 50 μM SO and increasing concentrations of RNAse A/T1. Alternatively, aggregated proteins were dissolved in 2% SDS, 8M Urea, 50 mM Tris-HCl pH7.5 by sonication and analysed by western blot.

### Ribonucleoside Vanadyl: Impact on Protein Aggregation.

Lysate prepared as before and incubated with 10 μM Ribonucleoside vanadyl (New England Biolabs). Aggregated proteins were isolated as described above and analysed by SDS-PAGE and western blotting.

### Example 14

TABLE 4
Annotation of RNA-seq peaks to various
genomic elements. Data are shown only
for the most prevalent annotations.

| | NfH | | Aβ | |
|---|---|---|---|---|
| | Number | % of | Number | % of |
| Annotation | of peaks | total | of peaks | total |
| Exon | 6441 | 36.0 | 5076 | 54.3 |
| Intron | 3943 | 22.0 | 1325 | 14.2 |
| LINE | 2661 | 14.9 | 624 | 6.7 |
| 3UTR | 1469 | 8.2 | 696 | 7.4 |
| Intergenic | 887 | 5.0 | 420 | 4.5 |
| Simple_repeat | 758 | 4.2 | 179 | 1.9 |
| SINE | 221 | 1.2 | 80 | 0.9 |
| LTR | 221 | 1.2 | 75 | 0.8 |
| Satellite | 216 | 1.2 | 54 | 0.6 |
| Promoter | 195 | 1.1 | 199 | 2.1 |
| Low_complexity | 110 | 0.6 | 78 | 0.8 |
| pseudo | 103 | 0.6 | 75 | 0.8 |
| ncRNA | 79 | 0.4 | 48 | 0.5 |
| 5UTR | 60 | 0.3 | 51 | 0.5 |
| rRNA | 10 | 0.1 | 59 | 0.6 |
| | PrP | | Tau | |
| | Number | % of | Number | % of |
| Annotations | of peaks | total | of peaks | total |
| Exon | 484 | 6.1 | 4218 | 22.2 |
| Intron | 890 | 11.3 | 6148 | 32.4 |
| LINE | 1150 | 14.5 | 3539 | 18.6 |
| 3UTR | 15 | 0.2 | 1638 | 8.6 |
| Intergenic | 770 | 9.7 | 737 | 3.9 |
| Simple_repeat | 2276 | 28.8 | 262 | 1.4 |
| SINE | 872 | 11.0 | 752 | 4.0 |
| LTR | 97 | 1.2 | 357 | 1.9 |
| Satellite | 625 | 7.9 | 47 | 0.2 |
| Promoter | 146 | 1.8 | 230 | 1.2 |
| Low_complexity | 289 | 3.7 | 120 | 0.6 |
| pseudo | 15 | 0.2 | 90 | 0.5 |
| ncRNA | 24 | 0.3 | 92 | 0.5 |
| 5UTR | 10 | 0.1 | 103 | 0.5 |
| rRNA | 10 | 0.1 | 4 | 0.0 |

The data show that the majority of the peaks in the different samples are located in coding-(exons), intronic-, and repetitive regions. The majority of the peaks in repetitive regions are LINE, SINE and simple repeats elements. The peaks associated with the prion protein (PrP) differ from the others in that a higher proportion is derived from repeats, with a concomitant decrease in peaks in coding regions.

TABLE 5
Predicted binding motifs in RNA associated with soluble Abeta, NFH, PrP, and tau.

| Seq | | Seq | | Seq | | Seq | |
|---|---|---|---|---|---|---|---|
| No | Sequence | No | Sequence | No | Sequence | No | Sequence |
| 1 | DGVAGAA | 44 | CGATATTG | 87 | CCATHACC | 130 | KACACACG |
| 2 | TTCTBCH | 45 | CAGGWA | 88 | GGTDATGG | 131 | CTGTCTWA |
| 3 | CTGTA | 46 | TWCCTG | 89 | ACCCTAAC | 132 | TWAGACAG |
| 4 | TACAG | 47 | CGTGANC | 90 | GTTAGGGT | 133 | CATDTAAA |
| 5 | AARGAAR | 48 | GNTCACG | 91 | GCMTGTR | 134 | TTTAHATG |
| 6 | YTTCYTT | 49 | MATCGCCA | 92 | YACAKGC | 135 | CTCATACA |
| 7 | AAATAY | 50 | TGGCGATK | 93 | TCCATTCR | 136 | TGTATGAG |
| 8 | RTATTT | 51 | CGTA | 94 | YGAATGGA | 137 | CTGAGGYC |
| 9 | CATYTKC | 52 | TACG | 95 | CAGCCTGG | 138 | GRCCTCAG |
| 10 | GMARATG | 53 | CCCGAGTA | 96 | CCAGGCTG | 139 | ACCAYGCC |
| 11 | AGGCHGAG | 54 | TACTCGGG | 97 | TCCRTCCA | 140 | GGCRTGGT |
| 12 | CTCDGCCT | 55 | GGTGVTGA | 98 | TGGAYGGA | 141 | GRAGRA |
| 13 | CAGCMTGG | 56 | TCABCACC | 99 | AAABACAC | 142 | TYCTYC |
| 14 | CCAKGCTG | 57 | GNAGAAR | 100 | GTGTVTTT | 143 | CABCWTC |
| 15 | CACDG | 58 | YTTCTNC | 101 | CRGCCTCC | 144 | GAWGVTG |
| 16 | CHGTG | 59 | GANGAKGA | 102 | GGAGGCYG | 145 | TACSW |
| 17 | CATACTGB | 60 | TCMTCNTC | 103 | TCCCAGCW | 146 | WSGTA |
| 18 | VCAGTATG | 61 | CCACSW | 104 | WGCTGGGA | 147 | DGGAAR |
| 19 | ATGRTGGY | 62 | WSGTGG | 105 | ATGGGTAR | 148 | YTTCCH |
| 20 | RCCAYCAT | 63 | ARGAAR | 106 | YTACCCAT | 149 | CTGTA |
| 21 | GCTGGGAY | 64 | YTTCYT | 107 | GATAGTGA | 150 | TACAG |
| 22 | RTCCCAGC | 65 | CHGCHGC | 108 | TCACTATC | 151 | GCKGCKGC |
| 23 | CTAYAAA | 66 | GCDGCDG | 109 | CTACCATY | 152 | GCMGCMGC |
| 24 | TTTRTAG | 67 | DGTAY | 110 | RATGGTAG | 153 | RCCACCW |
| 25 | ATGCMCAC | 68 | RTACH | 111 | AGGTTGCM | 154 | WGGTGGY |
| 26 | GTGKGCAT | 69 | GAWGAW | 112 | KG CAACCT | 155 | MAGAAR |
| 27 | ACCCTAAC | 70 | WTCWTC | 113 | GGTSGTGA | 156 | YTTCTK |
| 28 | GTTAGGG | 71 | CTGKAR | 114 | TCACSACC | 157 | GCTGB |
| 29 | CAGYATC | 72 | YTMCAG | 115 | GHGTGAAC | 158 | VCAGC |
| 30 | GATRCTG | 73 | ACGK | 116 | GTTCACDC | 159 | AAATATDT |
| 31 | CMTGGGTA | 74 | MCGT | 117 | GCARTGGC | 160 | AHATATTT |
| 32 | TACCCAKG | 75 | KAAAAYA | 118 | GCCAYTGC | 161 | AHCGTG |
| 33 | RMATGGAA | 76 | TRTTTTM | 119 | AATGGAAK | 162 | CACGDT |
| 34 | TTCCATKY | 77 | GATGVC | 120 | MTTCCATT | 163 | ANATAC |
| 35 | ACCRCGCC | 78 | GBCATC | 121 | AGGCAGR | 164 | GTATNT |
| 36 | GGCGYGGT | 79 | CAGGTAA | 122 | YCTGCCT | 165 | CCRCCKCC |
| 37 | AGAATSTG | 80 | TTACCTG | 123 | AAAMATAC | 166 | GGMGGYGG |
| 38 | CASATTCT | 81 | CTCKGCC | 124 | GTATKTTT | 167 | CAGKATG |
| 39 | AKCCATCC | 82 | GGCMGAG | 125 | CACAAATR | 168 | CATMCTG |
| 40 | GGATGG MT | 83 | TCTATSCA | 126 | YATTTGTG | 169 | GCCATGK |
| 41 | GTCTCGAW | 84 | TGSATAGA | 127 | ATTCACMC | 170 | MCATGGC |
| 42 | WTCGAGAC | 85 | TAGDTGGA | 128 | GKGTGAAT | 171 | AGGCCGW |
| 43 | CAATATCG | 86 | TCCAHCTA | 129 | CGTGTGTM | 172 | WCGGCCT |

Motifs 1-56 are from Abeta, motifs 57-82 are from NFH, motifs 83-141 are from PrP, and motifs 142-172 are from tau. Motifs are shown in forward direction as DNA. The reverse complimentary motifs are as likely to be associated with the proteins as the shown forward motifs. RNA motifs are generated by substituting “T”s for “U”s. Non-A,T,G,C naming follows the IUPAC-IUB recommendations for nucleotide nomenclature.

TABLE 6
Predicted binding motifs in DNA associated with
soluble Abeta, PrP, and all proteins.

| Seq | | Seq | | Seq | |
|---|---|---|---|---|---|
| No: | Sequence | No: | Sequence | No: | Sequence |
| 1 | AHTACA | 129 | CGCGGTTA | 257 | CAGGCGTG |
| 2 | TGTADT | 130 | TAACCGCG | 258 | TAATTTT |
| 3 | AGGCKGR | 131 | AAATATCY | 259 | AAACCAGT |
| 4 | YCMGCCT | 132 | RGATATTT | 260 | AYTATAGG |
| 5 | CCYRGCTA | 133 | GAGTTGAA | 261 | ACTGGTTT |
| 6 | TAGCYRGG | 134 | TTCAACTC | 262 | CCTATART |
| 7 | CTCCTGMC | 135 | AGGTGAGC | 263 | GGTCTCCA |
| 8 | GKCAGGAG | 136 | GCTCACCT | 264 | TCTTGAA |
| 9 | CCAMCAYG | 137 | CACGTTGG | 265 | TGGAGACC |
| 10 | CRTGKTGG | 138 | CCAACGTG | 266 | TTCAAGA |
| 11 | GGKTTCAM | 139 | AHTACA | 267 | GCAARACC |
| 12 | KTGAAMCC | 140 | TAWTTTTW | 268 | RGGAGGCA |
| 13 | TACDAAA | 141 | TGTADT | 269 | GGTYTTGC |
| 14 | TTTHGTA | 142 | WAAAAWTA | 270 | TGCCTCCY |
| 15 | GTGAKCY | 143 | AGGCKGR | 271 | AAAAAAAA |
| 16 | RGMTCAC | 144 | YCMGCCT | 272 | CAKCCTGG |
| 17 | CSRTCTC | 145 | CCYRGCTA | 273 | CCAGGMTG |
| 18 | GAGAYSG | 146 | CTRTARTC | 274 | TTTTTTTT |
| 19 | GCCTCCCR | 147 | GAYTAYAG | 275 | AATCAGSC |
| 20 | YGGGAGGC | 148 | TAGCYRGG | 276 | ACCACCAC |
| 21 | CTYGAACY | 149 | AGTAGCTR | 277 | GSCTGATT |
| 22 | RGTTCRAG | 150 | CTCCTGMC | 278 | GTGGTGGT |
| 23 | GCCACYRC | 151 | GKCAGGAG | 279 | GAGGTCRR |
| 24 | GYRGTGGC | 152 | YAGCTACT | 280 | GGGTTTTR |
| 25 | AAGTGCTG | 153 | CCAMCAYG | 281 | YAAAACCC |
| 26 | CAGCACTT | 154 | CTCCTGMC | 282 | YYGACCTC |
| 27 | CAACCTCY | 155 | CRTGKTGG | 283 | AATCGC |
| 28 | RGAGGTTG | 156 | GKCAGGAG | 284 | CAAYATGG |
| 29 | AAATTADC | 157 | GGKTTCAM | 285 | CCATRTTG |
| 30 | GHTAATTT | 158 | RTAGAGAY | 286 | GCGATT |
| 31 | GGTYTCR | 159 | KTGAAMCC | 287 | ATMTTGGC |
| 32 | YGARACC | 160 | RTCTCTAY | 288 | CCCGARTA |
| 33 | GGSCAACA | 161 | RGKTTCA | 289 | GCCAAKAT |
| 34 | TGTTGSCC | 162 | TACDAAA | 290 | TAYTCGGG |
| 35 | TCTCRAA | 163 | TGAAMCY | 291 | AGGTGRGC |
| 36 | TTYGAGA | 164 | TTTHGTA | 292 | CGRGTGCC |
| 37 | AAAAT | 165 | CCAMCAYG | 293 | GCYCACCT |
| 38 | ATTTT | 166 | GTGAKCY | 294 | GGCACYCG |
| 39 | CRBGCGCC | 167 | CRTGKTGG | 295 | ACCGYGCC |
| 40 | GGCGCVYG | 168 | RGMTCAC | 296 | CRAGGCAG |
| 41 | AATGGMRT | 169 | CSRTCTC | 297 | CTGCCTYG |
| 42 | AYKCCATT | 170 | GTGAKCY | 298 | GGCRCGGT |
| 43 | ATYGCTTG | 171 | GAGAYSG | 299 | CTGTCRCC |
| 44 | CAAGCRAT | 172 | RGMTCAC | 300 | GAGGCAGR |
| 45 | AGTARCTG | 173 | GCCTCCCR | 301 | GGYGACAG |
| 46 | CAGYTACT | 174 | TCTYGAW | 302 | YCTGCCTC |
| 47 | CTGTCKC | 175 | WTCRAGA | 303 | CCRTGTTA |
| 48 | GMGACAG | 176 | YGGGAGGC | 304 | CGKGCACC |
| 49 | GCAATCBC | 177 | CTYGAACY | 305 | GGTGCMCG |
| 50 | GVGATTGC | 178 | GCCTCCCR | 306 | TAACAYGG |
| 51 | AGTGSART | 179 | RGTTCRAG | 307 | CMACCTCC |
| 52 | AYTSCACT | 180 | YGGGAGGC | 308 | TGAGCCCA |
| 53 | CCYGAG | 181 | GCCACYRC | 309 | GGAGGTKG |
| 54 | CTCRGG | 182 | GYRGTGGC | 310 | TGGGCTCA |
| 55 | AGTYTCRC | 183 | AAGTGCTG | 311 | CCACCTCG |
| 56 | GYGARACT | 184 | CCYGGCTA | 312 | CCAGCTAC |
| 57 | DCATGCC | 185 | CAGCACTT | 313 | CGAGGTGG |
| 58 | GGCATGH | 186 | TAGCCRGG | 314 | GTAGCTGG |
| 59 | ASRAGAAT | 187 | AAKTGCT | 315 | ATCAYGAG |
| 60 | ATTCTYST | 188 | CAACCTCY | 316 | GAGAAACC |
| 61 | CACGCCTG | 189 | AGCAMTT | 317 | CTCRTGAT |
| 62 | CAGGCGTG | 190 | RGAGGTTG | 318 | GGTTTCTC |
| 63 | AYTATAGG | 191 | AAATTADC | 319 | ACGGAGTC |
| 64 | CCTATART | 192 | AATCRCT | 320 | ACTGCAAS |
| 65 | TCTTGAA | 193 | AGYGATT | 321 | GACTCCGT |
| 66 | TTCAAGA | 194 | GHTAATTT | 322 | STTGCAGT |
| 67 | GCAARACC | 195 | GAG VTTGC | 323 | ACCTCCCR |
| 68 | GGTYTTGC | 196 | GGTYTCR | 324 | GAGKTCRA |
| 69 | AAAAAAAA | 197 | GCAABCTC | 325 | TYGAMCTC |
| 70 | TTTTTTTT | 198 | YGARACC | 326 | YGGGAGGT |
| 71 | ACCACCAC | 199 | GAAACCCY | 327 | AGACCAKC |
| 72 | GTGGTGGT | 200 | GGSCAACA | 328 | CGCGGTTA |
| 73 | GAGGTCRR | 201 | RGGGTTTC | 329 | GMTGGTCT |
| 74 | YYGACCTC | 202 | TGTTGSCC | 330 | TAACCGCG |
| 75 | AATCGC | 203 | TCTCAAA | 331 | AGCRATCC |
| 76 | GCGATT | 204 | TCTCRAA | 332 | CCCAASTA |
| 77 | ATMTTGGC | 205 | TTTGAGA | 333 | GGATYGCT |
| 78 | GCCAAKAT | 206 | TTYGAGA | 334 | TASTTGGG |
| 79 | CGRGTGCC | 207 | AAAAT | 335 | AAAAACAC |
| 80 | GGCACYCG | 208 | GGBCAACA | 336 | AAGATCGY |
| 81 | ACCGYGCC | 209 | ATTTT | 337 | GTGTTTTT |
| 82 | GGCRCGGT | 210 | TGTTGVCC | 338 | RCGATCTT |
| 83 | GAGGCAGR | 211 | CCCRGC | 339 | GAATGGAA |
| 84 | YCTGCCTC | 212 | CRBGCGCC | 340 | GTGACAGA |
| 85 | CGKGCACC | 213 | GCYGGG | 341 | TCTGTCAC |
| 86 | GGTGCMCG | 214 | GGCGCVYG | 342 | TTCCATTC |
| 87 | TGAGCCCA | 215 | AATGGMRT | 343 | CACCACCA |
| 88 | TGGGCTCA | 216 | CAGGCRYG | 344 | GRTTGCA |
| 89 | CCACCTCG | 217 | AYKCCATT | 345 | TGCAAYC |
| 90 | CGAGGTGG | 218 | CRYGCCTG | 346 | TGGTGGTG |
| 91 | GAGAAACC | 219 | ATYGCTTG | 347 | AAAMAAAA |
| 92 | GGTTTCTC | 220 | TGAGVYCA | 348 | ATCATGAG |
| 93 | ACTGCAAS | 221 | CAAGCRAT | 349 | CTCATGAT |
| 94 | STTGCAGT | 222 | TGRBCTCA | 350 | TTTTKTTT |
| 95 | ACCTCCCR | 223 | AGTARCTG | 351 | AAAASTAC |
| 96 | YGGGAGGT | 224 | DAAAATAC | 352 | ATCACTTG |
| 97 | AGACCAKC | 225 | CAGYTACT | 353 | CAAGTGAT |
| 98 | GMTGGTCT | 226 | GTATTTTH | 354 | GTASTTTT |
| 99 | CCCAASTA | 227 | CRGGCRC | 355 | AACAGCAC |
| 100 | TASTTGGG | 228 | CTGTCKC | 356 | GGCGCCC |
| 101 | AAGATCGY | 229 | GMGACAG | 357 | GGGCGCC |
| 102 | RCGATCTT | 230 | GYGCCYG | 358 | GTGCTGTT |
| 103 | GTGACAGA | 231 | GCAATCBC | 359 | ACTGTACT |
| 104 | TCTGTCAC | 232 | RAGATCGY | 360 | CASGTGCC |
| 105 | CACCACCA | 233 | GVGATTGC | 361 | AGTACAGT |
| 106 | TGGTGGTG | 234 | RCGATCTY | 362 | GGCACSTG |
| 107 | ATCATGAG | 235 | AGTGCART | 363 | CCAGCCAA |
| 108 | CTCATGAT | 236 | AGTGSART | 364 | GSCACCCA |
| 109 | ATCACTTG | 237 | AYTGCACT | 365 | TGGGTGSC |
| 110 | CAAGTGAT | 238 | AYTSCACT | 366 | TTGGCTGG |
| 111 | GGCGCCC | 239 | AATGGCG | 367 | AGCAAGAC |
| 112 | GGGCGCC | 240 | CCYGAG | 368 | GAGATCA |
| 113 | CASGTGCC | 241 | CGCCATT | 369 | GTCTTGCT |
| 114 | GGCACSTG | 242 | CTCRGG | 370 | TGATCTC |
| 115 | GSCACCCA | 243 | AGTYTCRC | 371 | ACTGTACT |
| 116 | TGGGTGSC | 244 | CDCCACCA | 372 | CAGGAGGC |
| 117 | GAGATCA | 245 | GYGARACT | 373 | AGTACAGT |
| 118 | TGATCTC | 246 | TGGTGGHG | 374 | GCCTCCTG |
| 119 | ACTGTACT | 247 | AGYGAGAC | 375 | CGAGCATC |
| 120 | AGTACAGT | 248 | DCATGCC | 376 | GTTTCAAA |
| 121 | GTTTCAAA | 249 | GGCATGH | 377 | GATGCTCG |
| 122 | TTTGAAAC | 250 | GTCTCRCT | 378 | TTTGAAAC |
| 123 | AATCAGGC | 251 | ASRAGAAT | 379 | AATCAGGC |
| 124 | GCCTGATT | 252 | GRATTACA | 380 | GGATTTCA |
| 125 | GATTKCAC | 253 | ATTCTYST | 381 | GCCTGATT |
| 126 | GTGMAATC | 254 | TGTAATYC | 382 | TGAAATCC |
| 127 | CRTGTTAG | 255 | AAAATTA | 383 | CACGKTGG |
| 128 | CTAACAYG | 256 | CACGCCTG | 384 | GATTKCAC |
| Seq | Sequence | Seq | Sequence | Seq | Sequence |
| 385 | CCAMCGTG | 513 | GGSCAACA | 641 | ACCGYGCC |
| 386 | GTGMAATC | 514 | RGGGTTTC | 642 | CRAGGCAG |
| 387 | ATKGTGCC | 515 | TGTTGSCC | 643 | CTGCCTYG |
| 388 | CRTGTTAG | 516 | TGTTGVCC | 644 | GGCRCGGT |
| 389 | CTAACAYG | 517 | ATCYTGGC | 645 | CTGTCRCC |
| 390 | GGCACMAT | 518 | TCTCAAA | 646 | GAGGCAGR |
| 391 | AAAAATT | 519 | TCTCRAA | 647 | GGYGACAG |
| 392 | CGCGGTTA | 520 | GCCARGAT | 648 | YCTGCCTC |
| 393 | AATTTTT | 521 | TTTGAGA | 649 | CCRTGTTA |
| 394 | TAACCGCG | 522 | TTYGAGA | 650 | CGKGCACC |
| 395 | AAAAATGC | 523 | AAAAT | 651 | GGTGCMCG |
| 396 | AAATATCY | 524 | AKCRAGAC | 652 | TAACAYGG |
| 397 | GCATTITT | 525 | GGBCAACA | 653 | CMACCTCC |
| 398 | RGATATTT | 526 | ATTTT | 654 | TGAGCCCA |
| 399 | ATGATCTY | 527 | GTCTYGMT | 655 | GGAGGTKG |
| 400 | GAGTTGAA | 528 | TGTTGVCC | 656 | TGGGCTCA |
| 401 | RAGATCAT | 529 | ATTAYAGG | 657 | CCACCTCG |
| 402 | TTCAACTC | 530 | CCCRGC | 658 | CCAGCTAC |
| 403 | AGGTGAGC | 531 | CRBGCGCC | 659 | CGAGGTGG |
| 404 | AGTGAACS | 532 | CCTRTAAT | 660 | GTAGCTGG |
| 405 | GCTCACCT | 533 | GCYGGG | 661 | ATCAYGAG |
| 406 | SGTTCACT | 534 | GGCGCVYG | 662 | GAGAAACC |
| 407 | CACGTTGG | 535 | AATGGMRT | 663 | CTCRTGAT |
| 408 | CCCAAGTA | 536 | CAGGCRYG | 664 | GGTTTCTC |
| 409 | CCAACGTG | 537 | GAAACYCC | 665 | ACGGAGTC |
| 410 | TACTTGGG | 538 | AYKCCATT | 666 | ACTGCAAS |
| 411 | CGTCTKTA | 539 | CRYGCCTG | 667 | GACTCCGT |
| 412 | TAMAGACG | 540 | GGRGTTTC | 668 | STTGCAGT |
| 413 | AGGCTGCA | 541 | ATYGCTTG | 669 | ACCTCCCR |
| 414 | TGCAGCCT | 542 | GCCTCCCR | 670 | GAGKTCRA |
| 415 | GCTCGCTA | 543 | TGAGVYCA | 671 | TYGAMCTC |
| 416 | TAGCGAGC | 544 | CAAGCRAT | 672 | YGGGAGGT |
| 417 | GTCTCCAC | 545 | TGRBCTCA | 673 | AGACCAKC |
| 418 | GTGGAGAC | 546 | YGGGAGGC | 674 | CGCGGTTA |
| 419 | CTCRTGCC | 547 | AGTARCTG | 675 | GMTGGTCT |
| 420 | GGCAYGAG | 548 | CCYGCCAC | 676 | TAACCGCG |
| 421 | CACCTCCC | 549 | DAAAATAC | 677 | AGCRATCC |
| 422 | GGGAGGTG | 550 | CAGYTACT | 678 | CCCAASTA |
| 423 | AGGCTGR | 551 | GTATTTTH | 679 | GGATYGCT |
| 424 | AHTACA | 552 | GTGGCRGG | 680 | TASTTGGG |
| 425 | TAWTTTTW | 553 | ARGTGATC | 681 | AAAAACAC |
| 426 | TGTADT | 554 | CRGGCRC | 682 | AAGATCGY |
| 427 | WAAAAWTA | 555 | CTGTCKC | 683 | GTGTTTTT |
| 428 | YCAGCCT | 556 | GATCACYT | 684 | RCGATCTT |
| 429 | AGGCKGR | 557 | GMGACAG | 685 | GAATGGAA |
| 430 | GGAYTACA | 558 | GYGCCYG | 686 | GTGACAGA |
| 431 | TGTARTCC | 559 | GCAATCBC | 687 | TCTGTCAC |
| 432 | YCMGCCT | 560 | GGTGGTGA | 688 | TTCCATTC |
| 433 | AAAWTTA | 561 | RAGATCGY | 689 | CACCACCA |
| 434 | CCYRGCTA | 562 | GVGATTGC | 690 | GRTTGCA |
| 435 | CTRTARTC | 563 | RCGATCTY | 691 | TGCAAYC |
| 436 | GAYTAYAG | 564 | TCACCACC | 692 | TGGTGGTG |
| 437 | TAAWTTT | 565 | AAAAAWA | 693 | AAAMAAAA |
| 438 | TAGCYRGG | 566 | AGTGCART | 694 | ATCATGAG |
| 439 | AGTAGCTR | 567 | AGTGSART | 695 | CTCATGAT |
| 440 | AGTAGM | 568 | AYTGCACT | 696 | TTTTKTTT |
| 441 | CTCCTGMC | 569 | AYTSCACT | 697 | AAAASTAC |
| 442 | GKCAGGAG | 570 | TWTTTTT | 698 | ATCACTTG |
| 443 | KCTACT | 571 | AATGGCG | 699 | CAAGTGAT |
| 444 | YAGCTACT | 572 | CAGGTTCA | 700 | GTASTTTT |
| 445 | CCAMCAYG | 573 | CCYGAG | 701 | AACAGCAC |
| 446 | CTCCTGMC | 574 | CGCCATT | 702 | GGCGCCC |
| 447 | CRTGKTGG | 575 | CTCRGG | 703 | GGGCGCC |
| 448 | GKCAGGAG | 576 | TGAACCTG | 704 | GTGCTGTT |
| 449 | ATGKTGGY | 577 | AGTYTCRC | 705 | ACTGTACT |
| 450 | GGKTTCAM | 578 | CDCCACCA | 706 | CASGTGCC |
| 451 | RTAGAGAY | 579 | GGGYGACA | 707 | AGTACAGT |
| 452 | KTGAAMCC | 580 | GYGARACT | 708 | GGCACSTG |
| 453 | RCCAMCAT | 581 | TGGTGGHG | 709 | CCAGCCAA |
| 454 | RTCTCTAY | 582 | TGTCRCCC | 710 | GSCACCCA |
| 455 | MAAATACA | 583 | AGYGAGAC | 711 | TGGGTGSC |
| 456 | RGKTTCA | 584 | ATCTCAGC | 712 | TTGGCTGG |
| 457 | TACDAAA | 585 | DCATGCC | 713 | AGCAAGAC |
| 458 | TGAAMCY | 586 | GCTGAGAT | 714 | GAGATCA |
| 459 | TGTATTTK | 587 | GGCATGH | 715 | GTCTTGCT |
| 460 | TTTHGTA | 588 | GTCTCRCT | 716 | TGATCTC |
| 461 | CCAMCAYG | 589 | ASRAGAAT | 717 | ACTGTACT |
| 462 | GGKTTCA | 590 | CACCYGGC | 718 | CAGGAGGC |
| 463 | GTGAKCY | 591 | GRATTACA | 719 | AGTACAGT |
| 464 | CRTGKTGG | 592 | ATTCTYST | 720 | GCCTCCTG |
| 465 | RGMTCAC | 593 | GCCRGGTG | 721 | CGAGCATC |
| 466 | TGAAMCC | 594 | TGTAATYC | 722 | GTTTCAAA |
| 467 | CSRTCTC | 595 | AAAATTA | 723 | GATGCTCG |
| 468 | GTGAKCCR | 596 | ATAGTGRT | 724 | TTTGAAAC |
| 469 | GTGAKCY | 597 | CACGCCTG | 725 | AATCAGGC |
| 470 | GAGAYSG | 598 | AYCACTAT | 726 | GGATTTCA |
| 471 | RGMTCAC | 599 | CAGGCGTG | 727 | GCCTGATT |
| 472 | YGGMTCAC | 600 | TAATTTT | 728 | TGAAATCC |
| 473 | CBGCCTCC | 601 | AAACCAGT | 729 | CACGKTGG |
| 474 | GCCTCCCR | 602 | AGAGAYGG | 730 | GATTKCAC |
| 475 | TCTYGAW | 603 | AYTATAGG | 731 | CCAMCGTG |
| 476 | GGAGGCVG | 604 | ACTGGTTT | 732 | GTGMAATC |
| 477 | WTCRAGA | 605 | CCRTCTCT | 733 | ATKGTGCC |
| 478 | YGGGAGGC | 606 | CCTATART | 734 | CRTGTTAG |
| 479 | AAKTGCT | 607 | CMACCTCC | 735 | CTAACAYG |
| 480 | CTYGAACY | 608 | GGTCTCCA | 736 | GGCACMAT |
| 481 | GCCTCCCR | 609 | TCTTGAA | 737 | AAAAATT |
| 482 | AGCAMTT | 610 | GGAGGTKG | 738 | CGCGGTTA |
| 483 | RGTTCRAG | 611 | TGGAGACC | 739 | AATTTTT |
| 484 | YGGGAGGC | 612 | TTCAAGA | 740 | TAACCGCG |
| 485 | GCCACYRC | 613 | GCAARACC | 741 | AAAAATGC |
| 486 | TCTCRAA | 614 | RGGAGGCA | 742 | AAATATCY |
| 487 | GYRGTGGC | 615 | GGTYTTGC | 743 | GCATTTTT |
| 488 | TTYGAGA | 616 | TGCCTCCY | 744 | RGATATTT |
| 489 | AAGTGCTG | 617 | AAAAAAAA | 745 | ATGATCTY |
| 490 | CCYGGCTA | 618 | CAKCCTGG | 746 | GAGTTGAA |
| 491 | CRYGCC | 619 | CCAGGMTG | 747 | RAGATCAT |
| 492 | CAGCACTT | 620 | TTTTTTTT | 748 | TTCAACTC |
| 493 | GGCRYG | 621 | AATCAGSC | 749 | AGGTGAGC |
| 494 | TAGCCRGG | 622 | ACCACCAC | 750 | AGTGAACS |
| 495 | AAKTGCT | 623 | GSCTGATT | 751 | GCTCACCT |
| 496 | AGVTTGCA | 624 | GTGGTGGT | 752 | SGTTCACT |
| 497 | CAACCTCY | 625 | GAGGTCRR | 753 | CACGTTGG |
| 498 | AGCAMTT | 626 | GGGTTTTR | 754 | CCCAAGTA |
| 499 | RGAGGTTG | 627 | YAAAACCC | 755 | CCAACGTG |
| 500 | TGCAABCT | 628 | YYGACCTC | 756 | TACTTGGG |
| 501 | AAATTADC | 629 | AATCGC | 757 | CGTCTKTA |
| 502 | AATCRCT | 630 | CAAYATGG | 758 | TAMAGACG |
| 503 | AGYGATT | 631 | CCATRTTG | 759 | AGGCTGCA |
| 504 | GHTAATTT | 632 | GCGATT | 760 | TGCAGCCT |
| 505 | AGTGCART | 633 | ATMTTGGC | 761 | GCTCGCTA |
| 506 | GAGVTTGC | 634 | CCCGARTA | 762 | TAGCGAGC |
| 507 | GGTYTCR | 635 | GCCAAKAT | 763 | GTCTCCAC |
| 508 | AYTGCACT | 636 | TAYTCGGG | 764 | GTGGAGAC |
| 509 | GCAABCTC | 637 | AGGTGRGC | 765 | CTCRTGCC |
| 510 | YGARACC | 638 | CGRGTGCC | 766 | GGCAYGAG |
| 511 | GAAACCCY | 639 | GCYCACCT | 767 | CACCTCCC |
| 512 | GGBCAACA | 640 | GGCACYCG | 768 | GGGAGGTG |

Motifs predicted using the MEME-ChIP program suite from sequenced DNA associated with soluble Abeta, the prion protein (PrP), or a mixture of refolded proteins. Motifs 1-138 are from Abeta, motifs 139-421 are from PrP, and motifs 422-768 are from all soluble proteins.

The last category ‘all soluble proteins’ refers to aggregated proteins refolded with genomic DNA and then, after removal of aggregated proteins, these proteins were captured by binding to a nitrocellulose membrane. As nucleic acids show very weak binding to nitrocellulose membranes, most of the isolated DNA will have been associated with the proteins. Motifs are shown in the forward direction as DNA. The reverse complimentary motifs are as likely to be associated with the proteins as the shown forward motifs. Non-A,T,G,C naming follows the IUPAC-IUB recommendations for nucleotide nomenclature.

TABLE 7
Sequences of synthetic oliqomers based on
predicted motifs or random controls.

| Oligo name | Sequence |
|---|---|
| Motif 1 | GCTAATTTTTGTATTTTTAGTAGCTAATTTTTGTATTT |
| Forward | TTAGTAGCTAATTTTTGTATTTTTAGTA GCTAATTTT |
| | TGTATTTTTAGTA |
| | [SEQ ID NO: 3] |
| Motif 1 | TACTAAAAATACAAAAATTAGCTACTAAAAATACAAAA |
| Reverse | ATTAGCTACTAAAAATACAAAAATTAGCTACTAAAAAT |
| | ACAAAAATTAGC |
| | [SEQ ID NO: 4] |
| Control 1 | GAGTAAGAATCTATTATATATGGAGTAAGAATCTATTA |
| Forward | TATATGGAGTAAGAATCTATTATATATGGAGTAAGAAT |
| | CTATTATATATG |
| | [SEQ ID NO: 5] |
| Control 1 | CATATATAATAGATTCTTACTCCATATATAATAGATTC |
| Reverse | TTACTCCATATATAATAGATTCTTACTCCATATATAAT |
| | AGATTCTTACTC |
| | [SEQ ID NO: 6] |
| Motif 2 | GGTGAGTGTGAGGGTGGGTGAGTGTGAGGGTGGGTGAG |
| Forward | TGTGAGGGTGGGTGAGTGTGAGGGTG |
| | [SEQ ID NO: 7] |
| Motif 2 | CACCCTCACACTCACCACCCTCACACTCACCCACCCTC |
| Reverse | ACACTCAC CCACCCTCACACTCACC |
| | [SEQ ID NO: 8] |
| Control 2 | ACTACAACGGGCCCGGCCCAATCACAGCTCGAGCGCCT |
| Forward | TGAATGACGTACTCATCTCTATGCAT |
| | [SEQ ID NO: 9] |
| Control 2 | ATGCATAGAGATGAGTACGTCATTCAAGGCGCTCGAGC |
| | TGTGATTGGGCCGGGCCCGTTGTAGT |
| | [SEQ ID NO: 10] |

### Additional Materials and Methods (Pertaining to Example 14)

### RNA-IP and Sequencing 2

The same procedure as described in “RNA immunoprecipitation and sequencing” was followed. Briefly, aggregated proteins from human neurons were refolded with pre-fragmented total RNA from Jurkat T-cells as described, and soluble proteins were subjected to immunoprecipitation using antibodies against Abeta (clone 4G8, #9220-02, Signet), PrP (Proteintech, #12555-1-AP), NFH (Covance, #SMI-32), tau (Sigma, #T9450), or GFP (Abcam). Antibodies were captured and washed as described before and eluted from the beads by the addition of 6 M Guanidine Thiocyanate. RNA was isolated from the eluate by Trizol LS extraction according to the manufacturers recommendations (Life Technologies) and dissolved in water. Equal volumes from each sample were used to generate Illumina sequencing libraries using the NEBNext® Ultra™ Directional RNA Library Prep Kit for Illumina® (NEB). Libraries were sequenced on the NextSeq platform. Two independent experiments were performed for each IP.

### ChIP-Seq

Aggregated proteins from human neurons were refolded with pre-fragmented genomic DNA from Jurkat T-cells as described, and soluble proteins were subjected to immunoprecipitation using antibodies against Abeta (clone 4G8, #9220-02, Signet), PrP (Proteintech, #12555-1-AP), or mouse/rabbit IgG (Sigma). Antibodies were captured and washed as described before and eluted from the beads by the addition of 1% SDS. Alternatively, soluble refolded proteins were passed through a nitrocellulose membrane (0.2 μm pore size, GE Healthcare), and then washed and eluted in 1% SDS. As a control, genomic DNA without any proteins was passed through nitrocellulose membranes. DNA from either the immunoprecipitation or the membrane capture procedure was isolated from the eluate by phenol extraction and dissolved in water. Equal volumes from each sample were used to generate Illumina sequencing libraries using the NEBNext® ChIP-Seq Library Prep Master Mix Set for Illumina® (NEB). Libraries were sequenced on the MiSeq platform. Two independent experiments were performed for each IP/filter.

### Fragmentation of DNA or RNA

Total RNA used for the RNA-IP and sequencing 2 above was fragmented by incubation in a solution containing 30 mM Mg²⁺ at 95° C. for 9 min. This was then supplemented with EDTA to a final concentration of 50 mM and the RNA isolated with Agencourt XP beads and eluted in 1×TE buffer. This procedure resulted in an average fragment length of 150 nt. Genomic DNA was fragmented by sonication using a Bioruptor (Diagenode). The average fragment length after fragmentation was 250 bp.

### Bioinformatic Analysis

Reads from the RNA-IP experiments were aligned to the human genome (hg19) using Star software with default settings. The proportion of reads mapping to various genomic regions was determined using HTSeq. Binding sites/peaks were determined by combining the two biological replicates using MACS2 software, allowing for duplicates. Peak enrichment was determined against reads from the GFP immunoprecipitations. The MEME-ChIP suite of software (http://meme-suite.org/tools/meme-chip) was used to predict enriched motifs, using a fragment size of 300 bp centred at the peaks identified by MACS2. Reads from ChIP-seq experiments were aligned to the human genome (hg19) using Bowtie2. Binding sites/peaks were determined by combining the two biological replicates using the MACS2 software, without any duplicates. Peak enrichment was determined against reads from the IgG immunoprecipitations or from the nitrocellulose filter without any proteins present. The MEME-ChIP suite of software was used to predict enriched motifs, using a fragment size of 300 bp centred at the peaks identified by MACS2. Annotation to various genomic regions was performed using the Homer software suite.

### Divalent Ions

Jurkat T-cell lysate was prepared as described and supplemented with various amounts of divalent ions (MgCl₂, CaCl₂, or ZnCl₂) followed by incubation for one hour at 37° C. Aggregated proteins were isolated as described and quantified using the BCA kit from Pierce. Alternatively, aggregated proteins were refolded with genomic DNA or total RNA from Jurkat T-cells and the soluble fraction supplemented with various concentrations of MgCl₂ followed by incubation for one hour at 37° C. Aggregated proteins were isolated as described and quantified using the BCA kit from Pierce.

Refolding of Proteins with Small Molecules or Synthetic DNA/RNA

A two-step refolding protocol was used to test the solubilising effect of small molecules or synthetic DNA or RNA. Briefly, aggregated or recombinant proteins were dissolved and diluted to 1-5 μg/μl in 6M GuHCl, 100 mM DTT, 3 mM EDTA, 20 mM Tris-HCl pH 7.4, 1% Triton X-100. 6 μl of this was added to tubes on ice and various amounts of sodium orthovanadate (SO), (NH4)[VO(O2)2(phen)]*2H2O (Comp 6), or DNA or RNA diluted in 54 μl 100 mM Tris-HCl pH 7.4 (SO and Comp 6) or TE (DNA and RNA), were added. The tubes were mixed by vortexing and incubated on ice for 5 min. 540 μl of Tris-HCl pH 7.4 was then added and the tubes mixed again by vortexing and incubated for 10 min on ice. This procedure results in a final dilution of 1:100, decreasing the concentration of GuHCl to 60 mM. The tubes were then incubated for one hour at 37° C., with shaking at 1000 rpm. Aggregated and soluble proteins were separated and analysed as described before. Synthetic DNA or RNA was heat-denatured for 2 min at 95° C. followed by cooling on ice before addition to the proteins.

### Generation of Synthetic DNA or RNA

A consensus sequence of human AluSx SINE repeats was obtained from Repbase (http://www.girinst.org/repbase/) and synthesised as a double stranded DNA oligomer. This fragment was cloned into a modified pCR II vector (Life Technologies) containing an introduced Nhe I site in the MSC, by opening the vector with Apa I and Nhe I. Isolated plasmids were sequence-verified and linearised by Nhe I cleavage and used for T7-mediated RNA transcription using the HiScribe™ T7 High Yield RNA Synthesis Kit from NEB according to the manufacturers instructions. After incubation, the samples were treated with DNAse I for 15 min at 37° C. and the RNA isolated with Agencourt XP beads. Synthetic DNA oligomers, based on identified motifs and controls, were synthesised by Integrated DNA Technologies and dissolved in water to a final concentration of 100 μM. The sequence of the consensus AluSx repeat is as follows:

[SEQ ID NO: 11]
ggccgggcgcggtggctcacgcctgtaatcccagcactttgggaggccg
aggcgggcggatcacctgaggtcaggagttcgagaccagcctggccaac
atggtgaaaccccgtctctactaaaaatacaaaaattagccgggcgtgg
tggcgcgcgcctgtaatcccagctactcgggaggctgaggcaggagaat
cgcttgaacccgggaggcggaggttgcagtgagccgagatcgcgccact
gcactccagcctgggcgacagagcgagactccgtctca

And when transcribed as RNA:

[SEQ ID NO: 12]
ggccgggcgcggUggcUcacgccUgUaaUcccagcacUUUgggaggccg
aggcgggcggaUcaccUgaggUcaggagUUcgagaccagccUggccaac
aUggUgaaaccccgUcUcUacUaaaaaUacaaaaaUUagccgggcgUgg
UggcgcgcgccUgUaaUcccagcUacUcgggaggcUgaggcaggagaaU
cgcUUgaacccgggaggcggaggUUgcagUgagccgagaUcgcgccacU
gcacUccagccUgggcgacagagcgagacUccgUcUca

### Production and Isolation of Recombinant TDP-43

Wild type human TDP-43 was amplified from cDNA and cloned into a bacterial expression vector in frame with a C-terminal His tag. A single clone was cultivated in LB medium until OD₆₀₀ was 0.6, at which point IPTG (0.5 mM final concentration) was added to induce TDP-43 expression. Cultures were left overnight at 18° C. Bacteria were collected by centrifugation and His-tagged TDP-43 purified using the Ni-NTA Spin Columns Kit from Qiagen according to the manufacturers instructions, using the supplied GuHCl lysis solution. Purity of isolated proteins was determined by SDS-PAGE electrophoresis and coomassie blue staining. Recombinant TDP-43 was quantified using the BCA kit (Pierce).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, protein aggregation or related fields are intended to be within the scope of the following claims.

1. A method for treating and/or preventing a disease associated with protein aggregation which comprises the step of preventing protein aggregation associated with RNA removal, by stabilising RNA; or reversing protein aggregation associated with RNA removal, by effectively replacing removed RNA.

2. A method according to claim 1 wherein the RNA is stabilised by altering ion balance in the cell.

3. A method according to claim 1 wherein the RNA is effectively replaced by adding RNA, DNA or LNA.

4. A method according to claim 3 wherein the RNA is ribosomal RNA.

5. A method according to claim 3 or 4 wherein the RNA, DNA or LNA comprises a G-quadruple structure (G4).

6. A method according to claim 1 wherein the RNA is effectively replaced by sodium orthovanadate, or a derivative, structural mimic or modified version thereof.

7. A method according to any preceding claim wherein the disease is type II diabetes; cancer; inclusion body myositis/myopathy; medullary carcinoma of the thyroid, atrial amyloidosis, hereditary cerebral haemorrhage with amyloidosis, pituitary prolactinoma, injection-localised amyloidosis, aortic medial amyloidosis, hereditary lattice corneal dystrophy, corneal amyloidosis associated with trichiasis, cataract, calcifying epithelial odontogenic tumour, pulmonary alveolar proteinosis, cutaneous lichen amyloidosis, a nonneuropathic systemic amyloidosis, or a neurodegenerative disease such as Alzheimer's disease, motor neuron disease (MND), Parkinson's disease, frontotemporal dementia, amyloidosis lateral sclerosis, Huntington's disease, spinocerebellar ataxias, spinocerebellar ataxia, spinal and bulbar muscular atrophy, denatotrubal-pallidoluysian atrophy, familial British dementia, familial Danish dementia and prion diseases.

8. A method for diagnosing a disease associated with protein aggregation which comprises the step of determining the level of effective RNA in a sample from a subject, wherein decreased effective RNA compared with an equivalent sample from a control subject indicates that the subject has, or is at risk of, a disease associated with protein aggregation.

9. A method for determining if a subject is at risk of developing a disease associated with protein aggregation which method comprises the step of determining the level of effective RNA in a sample from the subject, wherein decreased effective RNA compared with an equivalent sample from a control subject indicates that the subject has, or is at risk of, a disease associated with protein aggregation.

10. A method according to claim 8 or 9 wherein the RNA is ribosomal RNA.

11. A method according to any one of claims 8 to 10 wherein the RNA comprises G quadruple structures.

12. A method according to any one of claims 8 to 11 wherein the decrease in effective RNA is due to RNA degradation.

13. A method according to any one of claims 8 to 12 wherein the sample is a serum, plasma, cerebrospinal fluid sample or a tissue sample such as a brain, pancreatic or muscle sample.

14. A method according to any one of claims 8 to 13 wherein the disease is type II diabetes; cancer; inclusion body myositis/myopathy; medullary carcinoma of the thyroid, atrial amyloidosis, hereditary cerebral haemorrhage with amyloidosis, pituitary prolactinoma, injection-localised amyloidosis, aortic medial amyloidosis, hereditary lattice corneal dystrophy, corneal amyloidosis associated with trichiasis, cataract, calcifying epithelial odontogenic tumour, pulmonary alveolar proteinosis, cutaneous lichen amyloidosis, a nonneuropathic systemic amyloidosis, or a neurodegenerative disease such as Alzheimer's disease, motor neuron disease (MND), Parkinson's disease, frontotemporal dementia, amyloidosis lateral sclerosis, Huntington's disease, spinocerebellar ataxias, spinocerebellar ataxia, spinal and bulbar muscular atrophy, denatotrubal-pallidoluysian atrophy, familial British dementia, familial Danish dementia and prion diseases.

15. An animal model for a disease associated with protein aggregation, in which animal protein aggregation is induced by removal of RNA in a cell in the animal.

16. An animal model according to claim 15 wherein the RNA is ribosomal RNA.

17. An animal model according to claim 15 or claim 16 wherein the RNA is removed by inducing RNA degradation.

18. An animal model according to claim 17 wherein the RNA degradation is caused by administration of, or increasing the expression or activity of, an RNA ribonuclease.

19. An animal model according to claim 18 wherein the ribonuclease comprises RNase A, RNase T1 and/or RNase 1f.

20. An animal model according to any one of claims 15 to 17 wherein the effective amount of RNA is reduced by administration of antisense RNA or siRNA.

21. An animal model according to any one of claims 15 to 17 wherein the effective amount of RNA is reduced by inducing a reduction in RNA expression.

22. An animal model for a disease associated with protein aggregation, in which animal protein aggregation is induced using ribonucleoside vanadyl and/or divalent ions.

23. The use of ribonucleoside vanadyl to initiate the aggregation of a plurality of proteins in a cell or cell lysate.

24. The use according to according to claim 23 wherein at least one protein in the plurality of proteins is implicated in the pathogenesis of a disease associated with protein aggregation.

25. The use according to claim 24, wherein the disease is Type II diabetes; Inclusion body myositis/myopathy; or a neurodegenerative disease such as Alzheimer's disease, motor neuron disease (MND), Parkinson's disease, frontotemporal dementia and prion diseases.

26. The use according to any one of claims 23 to 25, wherein the plurality of proteins comprises at least one of the following: amyloid-β, MAPT, SCNA, TARDBP, FUS, HTT, PrP, Neurofilaments (NF-H) and alpha-synuclein.

27. An in vitro method for promoting the folding of a protein which comprises the step of contacting an unfolded or partially folded protein with RNA or genomic DNA in order to promote folding.

28. A method according to claim 27, wherein the method is carried out in an isolated cell, optionally wherein the isolated cell is an isolated bacterial cell or an isolated mammalian cell.

29. A use of RNA or DNA to promote the in vitro folding of an unfolded or partially unfolding protein.

30. A method or use according to any one of claims 27-29 wherein the RNA or genomic DNA comprises a G-quadruple structure.

31. A method according to claims 27, 28 or 30 or a use according to claim 29 or 30 wherein the protein is a transmembrane protein.

32. A method according to claims 27, 28 or 30 or a use according to claim 29 or 30 wherein the protein is a therapeutic protein or biological reagent.

33. A method according to claim 32, wherein the protein is an enzyme, antibody, protein ligand, receptor, structural protein or cofactor.


SEQUENCE LISTING (12 sequences)

SEQ ID NO: 1 - DNA, 40 bp, Artificial Sequence
  Oligo 18S 1129-69
gggcagcttccgggaaaccaaagtctttgggttccggggg

SEQ ID NO: 2 - DNA, 40 bp, Artificial Sequence
  Oligo No 4G 1129-69
ttaagggcatctgatgcggcgcgtgacgcggcgctaagtg

SEQ ID NO: 3 - DNA, 88 bp, Artificial Sequence
  Synthetic oligomer Motif 1 Forward
gctaatttttgtatttttagtagctaatttttgtatttttagtagctaatttttgtatttttagtagctaatttttgtatttttagta

SEQ ID NO: 4 - DNA, 88 bp, Artificial Sequence
  Synthetic oligomer Motif 1 Reverse
tactaaaaatacaaaaattagctactaaaaatacaaaaattagctactaaaaatacaaaaattagctactaaaaatacaaaaattagc

SEQ ID NO: 5 - DNA, 88 bp, Artificial Sequence
  Synthetic oligomer Control 1 Forward
gagtaagaatctattatatatggagtaagaatctattatatatggagtaagaatctattatatatggagtaagaatctattatatatg

SEQ ID NO: 6 - DNA, 88 bp, Artificial Sequence
  Synthetic oligomer Control 1 Reverse
catatataatagattcttactccatatataatagattcttactccatatataatagattcttactccatatataatagattcttactc

SEQ ID NO: 7 - DNA, 64 bp, Artificial Sequence
  Synthetic oligomer Motif 2 Forward
ggtgagtgtgagggtgggtgagtgtgagggtgggtgagtgtgagggtgggtgagtgtgagggtg

SEQ ID NO: 8 - DNA, 63 bp, Artificial Sequence
  Synthetic oligomer Motif 2 Reverse
caccctcacactcaccaccctcacactcacccaccctcacactcacccaccctcacactcacc

SEQ ID NO: 9 - DNA, 64 bp, Artificial Sequence
  Synthetic oligomer Control 2 Forward
actacaacgggcccggcccaatcacagctcgagcgccttgaatgacgtactcatctctatgcat

SEQ ID NO: 10 - DNA, 64 bp, Artificial Sequence
  Synthetic oligomer Control 2 Reverse
atgcatagagatgagtacgtcattcaaggcgctcgagctgtgattgggccgggcccgttgtagt

SEQ ID NO: 11 - DNA, 283 bp, Artificial Sequence
  Consensus AluSx repeat sequence
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccgggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctca

SEQ ID NO: 12 - DNA, 283 bp, Artificial Sequence
  RNA consensus AluSx repeat sequence transcribed
ggccgggcgcgguggcucacgccuguaaucccagcacuuugggaggccgaggcgggcggaucaccugaggucaggaguucgagaccagccuggccaacauggugaaaccccgucucuacuaaaaauacaaaaauuagccgggcgugguggcgcgcgccuguaaucccagcuacucgggaggcugaggcaggagaaucgcuugaacccgggaggcggagguugcagugagccgagaucgcgccacugcacuccagccugggcgacagagcgagacuccgucuca